SEQUENCE LISTING (9 sequences)

SEQ ID NO: 1 - protein, 8 aa, synthetic construct
  REGION 1..8: note = Synthetic peptide
GFVFSSYD

SEQ ID NO: 2 - protein, 8 aa, synthetic construct
  REGION 1..8: note = Synthetic peptide
ISSGGGIT

SEQ ID NO: 3 - protein, 13 aa, synthetic construct
  REGION 1..13: note = Synthetic peptide
AAHYFGSSGPFAY

SEQ ID NO: 4 - protein, 6 aa, synthetic construct
  REGION 1..6: note = Synthetic peptide
ENIFSY

SEQ ID NO: 5 - protein, 9 aa, synthetic construct
  REGION 1..9: note = Synthetic peptide
QHHYGTPFT

SEQ ID NO: 6 - protein, 121 aa, synthetic construct
  REGION 1..121: note = Synthetic peptide
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYVVGQGTLVTVSS

SEQ ID NO: 7 - protein, 106 aa, synthetic construct
  REGION 1..106: note = Synthetic peptide
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEI

SEQ ID NO: 8 - protein, 448 aa, synthetic construct
  REGION 1..448: note = Synthetic peptide
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNYNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 213 aa, synthetic construct
  REGION 1..213: note = Synthetic peptide
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0154250 A1
(43) Pub. Date: May 15, 2025

(54) CEACAM5 ADC-ANTI-PD1/PD-L1 COMBINATION THERAPY

(71) Applicant: SANOFI, Paris (FR)

(72) Inventors: Mustapha CHADJAA, Paris (FR); Nathalie LE BAIL, Paris (FR); Christine SOUFFLET, Paris (FR); Phillip DENNIS, Cambridge, MA (US); Samira BENSFIA, Cambridge, MA (US)

(73) Assignee: SANOFI, Paris, FR (FR)

(21) Appl. No.: 18/715,558

(22) PCT Filed: Dec. 1, 2022
(86) PCT No.: PCT/EP2022/084105

§ 371 Date: May 31, 2024

Related U.S. Application Data
(60) Provisional application No. 63/383,337, filed on Nov. 11, 2022.

(30) Foreign Application Priority Data
Dec. 2, 2021 (EP) 21306690.5

(51) Int. Cl. C07K 16/28; A61K 39/00; A61K 47/68; A61P 35/00

(52) U.S. Cl. CPC C07K 16/2803; A61K 47/6803; A61K 47/6853; A61P 35/00; C07K 16/2818; A61K 2039/505; C07K 2317/24; C07K 2317/94

(57) ABSTRACT

Provided are methods for combination treatment of a cancer using an antibody-drug conjugate (ADC) targeting CEACAM5 and an anti-PD-1 antibody or anti-PD-L1 antibody, optionally together with a platinum-containing agent such as cisplatin or carboplatin with or without pemetrexed. In certain embodiments the ADC is tusamitamab ravtansine. In certain embodiments the anti-PD-1 antibody is pembrolizumab. In certain embodiments, the cancer is nonsquamous non-small cell lung cancer (NSQ NSCLC). In certain embodiments, the cancer is advanced or metastatic NSQ NSCLC.

### REFERENCE TO SEQUENCE LISTING

The instant application contains a Sequence Listing which has been submitted electronically in .xml format and is hereby incorporated by reference in its entirety. Said .xml copy, created on Nov. 29, 2022, is named PR94313_S286_WO_SANOFI.xml.

### FIELD

The present disclosure relates to the field of therapeutic treatment of cancers that express CEACAM5. Certain aspects of the disclosure relate to combination therapies with immunoconjugates comprising an anti-CEACAM5 antibody with anti-PD-1 or anti-PD-L1 agents, to treat cancer, including lung, gastric, gastroesophageal junction, and esophageal cancer.

### BACKGROUND

The mechanism of action of antibody drug conjugates (ADCs) begins with its binding to a specific antigen, sufficiently expressed on the tumor cells in order to achieve a selective and efficient internalization of the drug. Selectively targeting potent cytotoxics to tumor cells using ADCs has now been shown to be an effective strategy for the treatment of cancer, as demonstrated by the recent approvals of brentuximab vedotin for the treatment of Hodgkin lymphoma and trastuzumab emtansine (T-DM1) for the treatment of relapsed metastatic HER2+ breast cancer (Younes A, Gopal A K, Smith S E, Ansell S M, Rosenblatt J D, Savage K J, et al. Results of a pivotal phase II study of brentuximab vedotin for patients with relapsed or refractory Hodgkin's lymphoma. J Clin Oncol. 2012; 30 (18): 2183-9; Verma S, Miles D, Gianni L, Krop I E, Welslau M, Baselga J, et al. Trastuzumab emtansine for HER2-positive advanced breast cancer. N Engl J Med. 2012; 19:1783-91). Many other malignant diseases with unmet medical needs, such as solid tumor cancers, could benefit from such therapeutic options.

Lung cancer, for example, is an aggressive form of cancer that is accounts for hundreds of thousands of deaths in the United States. Unfortunately, it tends to recur after initial treatment and become more resistant to subsequent treatment. While multiple treatments have been utilized for the treatment of individuals with lung cancer, more effective treatments are needed.

Various treatments are in use for the treatment of lung cancer. First line therapy can include chemotherapy with platinum-containing agents. More recently, immune checkpoint inhibitors have been approved for use in the treatment of lung cancer. WO 2020/161214, the entire content of which is incorporated herein by reference, discloses use of anti-CEACAM5 immunoconjugates (ADCs) for treating lung cancer.

### SUMMARY

This disclosure provides methods for treating cancer using a combination therapy including an ADC that specifically binds CEACAM5. The combination therapy includes the ADC and an anti-PD-1 antibody or an anti-PD-L1 antibody, and optionally with a platinum-based agent with or without pemetrexed. In certain embodiments, the ADC is tusamitamab ravtansine.

An aspect of the disclosure is a method of treating a cancer, comprising administering to a subject in need thereof an effective amount of (i) an antibody-drug conjugate (ADC) comprising an anti-CEACAM5 antibody and (ii) an anti-PD-1 antibody or an anti-PD-L1 antibody, wherein the cancer expresses CEACAM5, thereby treating the cancer. In another aspect, the disclosure provides an antibody-drug conjugate (ADC) comprising an anti-CEACAM5 antibody for use in treating a cancer, wherein the ADC is suitable for use in combination with an anti-PD-1 antibody or an anti-PD-L1 antibody, wherein the cancer expresses CEACAM5, thereby treating the cancer.

An aspect of the disclosure is a combination of (i) an antibody-drug conjugate (ADC) comprising an anti-CEACAM5 antibody and (ii) an anti-PD-1 antibody or anti-PD-L1 antibody, in an effective amount, for use in the treatment of a cancer in a subject in need thereof, wherein the cancer expresses CEACAM5.

In certain embodiments, the anti-CEACAM5 antibody comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

In certain embodiments, the anti-CEACAM5 antibody comprises a variable domain of a heavy chain (VH) consisting of SEQ ID NO: 6 and a variable domain of a light chain (VL) consisting of SEQ ID NO: 7.

In certain embodiments, the anti-CEACAM5 antibody is tusamitamab.

In certain embodiments, the ADC comprises at least one cytotoxic agent.

In certain embodiments, the cytotoxic agent is selected from the group consisting of radioisotopes, protein toxins, small molecule toxins, and any combination thereof.

In certain embodiments, the small molecule toxin is selected from the group consisting of antimetabolites, DNA-alkylating agents, DNA-cross-linking agents, DNA-intercalating agents, anti-microtubule agents, topoisomerase inhibitors, and any combination thereof.

In certain embodiments, the anti-microtubule agent is selected from the group consisting of taxanes, vinca alkaloids, maytansinoids, colchicine, podophyllotoxin, griseofulvin, and any combination thereof.

In certain embodiments, the maytansinoid is selected from the group consisting of N2′-deacetyl-N2′-(3-mercapto-1-oxopropyl)-maytansine (DM1), N2′-deacetyl-N-2′(4-methyl-4-mercapto-1-oxopentyl)-maytansine (DM4), and any combination thereof.

In certain embodiments, the anti-CEACAM5-antibody is covalently attached via a cleavable or non-cleavable linker to the at least one cytotoxic agent.

In certain embodiments, linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

In certain embodiments, the ADC is characterized by a drug-to-antibody ratio (DAR) ranging from 1 to 10.

In certain embodiments, the ADC is tusamitamab ravtansine.

In certain embodiments, the cancer expresses CEACAM5 with moderate or high intensity defined by immunohistochemistry.

In certain embodiments, the cancer expresses CEACAM5 with moderate intensity (immunohistochemistry intensity ≥2+ in ≥1% to <50% of tumor cells).

In certain embodiments, the cancer expresses CEACAM5 with high intensity (immunohistochemistry intensity ≥2+ in ≥50% of tumor cells).

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g cholangiacarcinoma), prostate cancer, and skin cancer.

In certain embodiments, the cancer is gastric cancer, gastroesophageal junction cancer, or esophageal cancer.

In certain embodiments, the cancer is lung cancer.

In certain embodiments, the lung cancer is nonsquamous non-small cell lung cancer (NSQ NSCLC).

In certain embodiments, the subject has advanced or metastatic NSQ NSCLC.

In certain embodiments, the subject has NSQ NSCLC with no epidermal growth factor receptor (EGFR) sensitizing mutation or v-raf murine sarcoma viral oncogene homolog B1 (BRAF) mutation or anaplastic lymphoma kinase/c-ros oncogene 1 (ALK/ROS) alterations.

In certain embodiments, the subject has received no prior systemic chemotherapy for treatment of the cancer.

In certain embodiments, the anti-PD-1 antibody or anti-PD-L1 antibody is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the anti-PD-1 antibody or anti-PD-L1 antibody is an anti-PD-L1 antibody.

In certain embodiments, the anti-PD-L1 antibody is selected from the group consisting of atezolizumab, avelumab, and durvalumab.

In certain embodiments, the anti-PD-1 antibody or the anti-PD-L1 antibody and the ADC are administered sequentially.

In certain embodiments, the anti-PD-1 antibody or the anti-PD-L1 antibody is administered before the ADC.

In certain embodiments, the anti-PD-1 antibody or the anti-PD-L1 antibody and the ADC are administered simultaneously.

In certain embodiments, the anti-PD-1 antibody or the anti-PD-L1 antibody and

the ADC are administered to the subject for at least four cycles.

In certain embodiments, each cycle is about two to six weeks.

In certain embodiments, each cycle is about two weeks.

In certain embodiments, each cycle is about three weeks.

In certain embodiments, each cycle is about six weeks.

In certain embodiments, each tusamitamab ravtansine cycle is selected from the group consisting of: two weeks, three weeks, and four weeks.

In certain embodiments, each anti-PD-1 antibody or anti-PD-L1 antibody cycle is selected from the group consisting of: two weeks, three weeks, and six weeks.

In certain embodiments, the anti-PD-1 antibody or the anti-PD-L1 antibody is administered to the subject intravenously in a dose of about 200 mg to about 400 mg.

In certain embodiments, the anti-PD-1 antibody or the anti-PD-L1 antibody is administered to the subject intravenously in a dose selected from 200 mg, 350 mg, 360 mg, and 400 mg.

In certain embodiments, the tusamitamab ravtansine is administered to the subject intravenously in a dose of about 60 mg/m² to about 190 mg/m².

In certain embodiments, the tusamitamab ravtansine is administered to the subject intravenously in a dose of about 120 mg/m² to about 170 mg/m².

In certain embodiments, the anti-PD-1 antibody or the anti-PD-L1 antibody is administered to the subject intravenously in a dose of about 200 mg and the tusamitamab ravtansine is administered to the subject intravenously in a dose of about 120 mg/m² to about 170 mg/m².

In certain embodiments, the anti-PD-1 antibody or the anti-PD-L1 antibody and the tusamitamab ravtansine are administered once every three weeks.

In certain embodiments, the anti-PD-1 antibody or the anti-PD-L1 antibody is administered to the subject intravenously in a dose of about 400 mg and the tusamitamab ravtansine is administered to the subject intravenously in a dose of about 120 mg/m² to about 170 mg/m².

In certain embodiments, the anti-PD-1 antibody or the anti-PD-L1 antibody is administered once every six weeks and the tusamitamab ravtansine is administered once every three weeks.

In certain embodiments, the ADC is tusamitamab ravtansine and the anti-PD-1 antibody or the anti-PD-L1 antibody is pembrolizumab.

In certain embodiments, the anti-PD-1 antibody or the anti-PD-L1 antibody and the ADC are administered about once every three weeks, the ADC being tusamitamab ravtansine and being administered to the subject intravenously in a dose of about 120 mg/m².

In certain embodiments, the anti-PD-1 antibody or the anti-PD-L1 antibody and the ADC are administered about once every three weeks, the ADC being tusamitamab ravtansine and being administered to the subject intravenously in a dose of about 150 mg/m².

In certain embodiments, the anti-PD-1 antibody is administered to the subject intravenously in a dose of about 200 mg.

In some embodiment, a method or a use of the disclosure further comprises administering a platinum-based chemotherapy to the subject

An aspect of the disclosure is a method of treating a cancer, comprising administering to a subject in need thereof an effective amount of (i) an antibody-drug conjugate (ADC) comprising an anti-CEACAM5 antibody, (ii) an anti-PD-1 antibody or an anti-PD-L1 antibody, and (iii) a platinum-based chemotherapy, wherein the cancer expresses CEACAM5, thereby treating the cancer. In another aspect, the disclosure provides an antibody-drug conjugate (ADC) comprising an anti-CEACAM5 antibody for use in treating a cancer, wherein the ADC is suitable for use in combination with (i) an anti-PD-1 antibody or an anti-PD-L1 antibody and (ii) a platinum-based chemotherapy, wherein the cancer expresses CEACAM5, thereby treating the cancer.

An aspect of the disclosure is a combination of (i) an antibody-drug conjugate (ADC) comprising an anti-CEACAM5 antibody, (ii) an anti-PD-1 antibody or anti-PD-L1 antibody, and (iii) a platinum-based chemotherapy, in an effective amount, for use in the treatment of a cancer in a subject in need thereof, wherein the cancer expresses CEACAM5.

In certain embodiments, the anti-CEACAM5 antibody comprises a HCDR1 having the amino acid sequence of SEQ ID NO: 1, a HCDR2 having the amino acid sequence of SEQ ID NO: 2), a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

In certain embodiments, the anti-CEACAM5 antibody comprises a variable domain of a heavy chain (VH) consisting of SEQ ID NO: 6 and a variable domain of a light chain (VL) consisting of SEQ ID NO: 7.

In certain embodiments, the anti-CEACAM5 antibody is tusamitamab.

In certain embodiments, the ADC comprises at least one cytotoxic agent.

In certain embodiments, the cytotoxic agent is selected from the group consisting of radioisotopes, protein toxins, small molecule toxins, and any combination thereof.

In certain embodiments, the small molecule toxin is selected from the group consisting of antimetabolites, DNA-alkylating agents, DNA-cross-linking agents, DNA-intercalating agents, anti-microtubule agents, topoisomerase inhibitors, and any combination thereof.

In certain embodiments, the anti-microtubule agent is selected from the group consisting of taxanes, vinca alkaloids, maytansinoids, colchicine, podophyllotoxin, griseofulvin, and any combination thereof.

In certain embodiments, the maytansinoid is selected from the group consisting of N2′-deacetyl-N2′-(3-mercapto-1-oxopropyl)-maytansine (DM1), N2′-deacetyl-N-2′(4-methyl-4-mercapto-1-oxopentyl)-maytansine (DM4), and any combination thereof.

In certain embodiments, the anti-CEACAM5-antibody is covalently attached via a cleavable or non-cleavable linker to the at least one cytotoxic agent.

In certain embodiments, the linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

In certain embodiments, the ADC is characterized by a drug-to-antibody ratio (DAR) ranging from 1 to 10.

In certain embodiments, the ADC is tusamitamab ravtansine.

In certain embodiments, wherein the cancer expresses CEACAM5 with moderate or high intensity defined by immunohistochemistry.

In certain embodiments, the cancer expresses CEACAM5 with moderate intensity (e.g., immunohistochemistry intensity ≥2+ in ≥1% to <50% of tumor cells).

In certain embodiments, the cancer expresses CEACAM5 with high intensity (e/g/. immunohistochemistry intensity ≥2+ in ≥50% of tumor cells).

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiacarcinoma), prostate cancer, and skin cancer.

In certain embodiments, the cancer is gastric cancer, gastroesophageal junction cancer, or esophageal cancer.

In certain embodiments, the cancer is lung cancer.

In certain embodiments, the lung cancer is non-squamous non-small cell lung cancer (NSQ NSCLC).

In certain embodiments, the subject has NSQ NSCLC with no epidermal growth factor receptor (EGFR) sensitizing mutation or v-raf murine sarcoma viral oncogene homolog B1 (BRAF) mutation or anaplastic lymphoma kinase/c-ros oncogene 1 (ALK/ROS) alterations.

In certain embodiments, the subject has received no prior systemic chemotherapy for treatment of the cancer.

In certain embodiments, the anti-PD-1 antibody or anti-PD-L1 antibody is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the anti-PD-1 antibody or anti-PD-L1 antibody is an anti-PD-L1 antibody.

In certain embodiments, the anti-PD-L1 antibody is selected from the group consisting of atezolizumab, avelumab, and durvalumab.

In certain embodiments, the platinum-based chemotherapy is selected from the group consisting of cisplatin and carboplatin.

In certain embodiments, the anti-PD-1 antibody or the anti-PD-L1 antibody and the ADC are administered sequentially.

In certain embodiments, the anti-PD-1 antibody or the anti-PD-L1 antibody is administered before the ADC.

In certain embodiments, the anti-PD-1 antibody or the anti-PD-L1 antibody is administered before the ADC and the platinum-based chemotherapy.

In certain embodiments, the anti-PD-1 antibody or the anti-PD-L1 antibody and the ADC are administered simultaneously.

In certain embodiments, the anti-PD-1 antibody or the anti-PD-L1 antibody, the ADC, and the platinum-based chemotherapy are administered to the subject for at least four cycles.

In certain embodiments, each cycle is about two to six weeks.

In certain embodiments, each cycle is about two weeks.

In certain embodiments, each cycle is about three weeks.

In certain embodiments, each cycle is about six weeks.

In certain embodiments, each tusamitamab ravtansine cycle is selected from the group consisting of: two weeks, three weeks, and four weeks.

In certain embodiments, each anti-PD-1 antibody or anti-PD-L1 antibody cycle is selected from the group consisting of: two weeks, three weeks, and six weeks.

In certain embodiments, the anti-PD-1 antibody or the anti-PD-L1 antibody is administered to the subject intravenously in a dose of about 200 mg to about 400 mg.

In certain embodiments, the anti-PD-1 antibody or the anti-PD-L1 antibody is administered to the subject intravenously in a dose selected from 200 mg, 350 mg, 360 mg, and 400 mg.

In certain embodiments, the tusamitamab ravtansine is administered to the subject intravenously in a dose of about 60 mg/m² to about 190 mg/m².

In certain embodiments, the tusamitamab ravtansine is administered to the subject intravenously in a dose of about 120 mg/m² to about 170 mg/m².

In certain embodiments, the tusamitamab ravtansine is administered to the subject intravenously in a dose of about 120 mg/m².

In certain embodiments, the tusamitamab ravtansine is administered to the subject intravenously in a dose of about 150 mg/m².

In certain embodiments, the tusamitamab ravtansine is administered to the subject intravenously in a dose of about 170 mg/m².

In certain embodiments, the anti-PD-1 antibody or the anti-PD-L1 antibody is administered to the subject intravenously in a dose of about 200 mg and the tusamitamab ravtansine is administered to the subject intravenously in a dose of about 120 mg/m² to about 170 mg/m².

In certain embodiments, the anti-PD-1 antibody or the anti-PD-L1 antibody and the tusamitamab ravtansine are administered once every three weeks.

In certain embodiments, the anti-PD-1 antibody or the anti-PD-L1 antibody is administered to the subject intravenously in a dose of about 400 mg and the tusamitamab ravtansine is administered to the subject intravenously in a dose of about 120 mg/m² to about 170 mg/m².

In certain embodiments, the anti-PD-1 antibody or the anti-PD-L1 antibody is administered once every six weeks and the tusamitamab ravtansine is administered once every three weeks.

In certain embodiments, the method comprises administering cisplatin to the subject.

In certain embodiments, the cisplatin is administered intravenously to the subject in a dose from 38 mg/m² to 75 mg/m².

In certain embodiments, the cisplatin is administered intravenously to the subject in a dose of about 75 mg/m².

In certain embodiments, the method comprises administering carboplatin to the subject.

In certain embodiments, the carboplatin is administered intravenously to the subject in a dose of about (target AUC)×[(140−age)×(weight in kg)/serum creatinine (mg/dL)×72(×0.85 if female)+25], wherein the target AUC is from AUC 2.5 to AUC 5.

In certain embodiments, the target AUC is AUC 5.

In certain embodiments, the ADC is tusamitamab and the anti-PD-1 antibody is pembrolizumab.

In some embodiment, a method or a use of the disclosure further comprises administering pemetrexed to the subject.

An aspect of the disclosure is a method of treating a cancer, comprising administering to a subject in need thereof an effective amount of (i) an antibody-drug conjugate (ADC) comprising an anti-CEACAM5 antibody, (ii) an anti-PD-1 antibody or an anti-PD-L1 antibody, (iii) a platinum-based chemotherapy, and (iv) pemetrexed, wherein the cancer expresses CEACAM5, thereby treating the cancer. In another aspect, the disclosure provides an antibody-drug conjugate (ADC) comprising an anti-CEACAM5 antibody for use in treating a cancer, wherein the ADC is suitable for use in combination with (i) an anti-PD-1 antibody or an anti-PD-L1 antibody, (ii) a platinum-based chemotherapy, and (iii) pemetrexed, wherein the cancer expresses CEACAM5, thereby treating the cancer.

An aspect of the disclosure is a combination of (i) an antibody-drug conjugate (ADC) comprising an anti-CEACAM5 antibody, (ii) an anti-PD-1 antibody or anti-PD-L1 antibody, (iii) a platinum-based chemotherapy, and (iv) pemetrexed, in an effective amount, for use in the treatment of a cancer in a subject in need thereof, wherein the cancer expresses CEACAM5.

In certain embodiments, the anti-CEACAM5 antibody comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

In certain embodiments, the anti-CEACAM5 antibody comprises a variable domain of a heavy chain (VH) consisting of SEQ ID NO: 6 and a variable domain of a light chain (VL) consisting of SEQ ID NO: 7.

In certain embodiments, the anti-CEACAM5 antibody is tusamitamab.

In certain embodiments, the ADC comprises at least one cytotoxic agent.

In certain embodiments, the cytotoxic agent is selected from the group consisting of radioisotopes, protein toxins, small molecule toxins, and any combination thereof.

In certain embodiments, the small molecule toxin is selected from the group consisting of antimetabolites, DNA-alkylating agents, DNA-cross-linking agents, DNA-intercalating agents, anti-microtubule agents, topoisomerase inhibitors, and any combination thereof.

In certain embodiments, the anti-microtubule agent is selected from the group consisting of taxanes, vinca alkaloids, maytansinoids, colchicine, podophyllotoxin, griseofulvin, and any combination thereof.

In certain embodiments, the maytansinoid is selected from the group consisting of N2′-deacetyl-N2′-(3-mercapto-1-oxopropyl)-maytansine (DM1), N2′-deacetyl-N-2′(4-methyl-4-mercapto-1-oxopentyl)-maytansine (DM4), and any combination thereof.

In certain embodiments, the anti-CEACAM5-antibody is covalently attached via a cleavable or non-cleavable linker to the at least one cytotoxic agent.

In certain embodiments, the linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

In certain embodiments, the ADC is characterized by a drug-to-antibody ratio (DAR) ranging from 1 to 10.

In certain embodiments, the ADC is tusamitamab ravtansine.

In certain embodiments, the cancer expresses CEACAM5 with moderate or high intensity defined by immunohistochemistry.

In certain embodiments, the cancer expresses CEACAM5 with moderate intensity (immunohistochemistry intensity ≥2+ in ≥1% to <50% of tumor cells).

In certain embodiments, the cancer expresses CEACAM5 with high intensity (immunohistochemistry intensity ≥2+ in ≥50% of tumor cells).

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g cholangiacarcinoma), prostate cancer, and skin cancer.

In certain embodiments, the cancer is gastric cancer, gastroesophageal junction cancer or esophageal cancer.

In certain embodiments, the cancer is lung cancer.

In certain embodiments, the lung cancer is nonsquamous non-small cell lung cancer (NSQ NSCLC).

In certain embodiments, the subject has NSQ NSCLC with no epidermal growth factor receptor (EGFR) sensitizing mutation or v-raf murine sarcoma viral oncogene homolog B1 (BRAF) mutation or anaplastic lymphoma kinase/c-ros oncogene 1 (ALK/ROS) alterations.

In certain embodiments, the subject has received no prior systemic chemotherapy for treatment of the cancer.

In certain embodiments, the anti-PD-1 antibody or anti-PD-L1 antibody is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the anti-PD-1 antibody or anti-PD-L1 antibody is an anti-PD-L1 antibody.

In certain embodiments, the anti-PD-L1 antibody is selected from the group consisting of atezolizumab, avelumab, and durvalumab.

In certain embodiments, the platinum-based chemotherapy is selected from the group consisting of cisplatin and carboplatin.

In certain embodiments, the anti-PD-1 antibody or the anti-PD-L1 antibody and the ADC are administered sequentially.

In certain embodiments, the anti-PD-1 antibody or the anti-PD-L1 antibody is administered before the ADC.

In certain embodiments, the anti-PD-1 antibody or the anti-PD-L1 antibody is administered before the ADC, the platinum-based chemotherapy, and the pemetrexed.

In certain embodiments, the anti-PD-1 antibody or the anti-PD-L1 antibody and the ADC are administered simultaneously.

In certain embodiments, the anti-PD-1 antibody or the anti-PD-L1 antibody, the ADC, and the platinum-based chemotherapy are administered to the subject for at least four cycles.

In certain embodiments, each cycle is about two to six weeks.

In certain embodiments, each cycle is about two weeks.

In certain embodiments, each cycle is about three weeks.

In certain embodiments, each cycle is about six weeks.

In certain embodiments, each tusamitamab ravtansine cycle is selected from the group consisting of: two weeks, three weeks, and four weeks.

In certain embodiments, each anti-PD-1 antibody or anti-PD-L1 antibody cycle is selected from the group consisting of: two weeks, three weeks, and six weeks.

In certain embodiments, the anti-PD-1 antibody or the anti-PD-L1 antibody is administered to the subject intravenously in a dose of about 200 mg to about 400 mg.

In certain embodiments, the anti-PD-1 antibody or the anti-PD-L1 antibody is administered to the subject intravenously in a dose selected from 200 mg, 350 mg, 360 mg, and 400 mg.

In certain embodiments, the tusamitamab ravtansine is administered to the subject intravenously in a dose of about 60 mg/m² to about 190 mg/m².

In certain embodiments, the tusamitamab ravtansine is administered to the subject intravenously in a dose of about 120 mg/m² to about 170 mg/m².

In certain embodiments, the tusamitamab ravtansine is administered to the subject intravenously in a dose of about 120 mg/m².

In certain embodiments, the tusamitamab ravtansine is administered to the subject intravenously in a dose of about 150 mg/m².

In certain embodiments, the tusamitamab ravtansine is administered to the subject intravenously in a dose of about 170 mg/m².

In certain embodiments, the anti-PD-1 antibody or the anti-PD-L1 antibody is administered to the subject intravenously in a dose of about 200 mg and the tusamitamab ravtansine is administered to the subject intravenously in a dose of about 120 mg/m² to about 170 mg/m².

In certain embodiments, the anti-PD-1 antibody or the anti-PD-L1 antibody and the tusamitamab ravtansine are administered once every three weeks.

In certain embodiments, the anti-PD-1 antibody or the anti-PD-L1 antibody is administered to the subject intravenously in a dose of about 400 mg and the tusamitamab ravtansine is administered to the subject intravenously in a dose of about 120 mg/m² to about 170 mg/m².

In certain embodiments, the anti-PD-1 antibody or the anti-PD-L1 antibody is administered once every six weeks and the tusamitamab ravtansine is administered once every three weeks.

In certain embodiments, the method comprises administering cisplatin to the subject.

In certain embodiments, the cisplatin is administered intravenously to the subject in a dose from 38 mg/m² to 75 mg/m².

In certain embodiments, the cisplatin is administered intravenously to the subject in a dose of about 75 mg/m².

In certain embodiments, the method comprises administering carboplatin to the subject.

In certain embodiments, the carboplatin is administered intravenously to the subject in a dose of about (target AUC)×[(140−age)×(weight in kg)/serum creatinine (mg/dL)×72(×0.85 if female)+25], wherein the target AUC is from AUC 2.5 to AUC 5.

In certain embodiments, the target AUC is AUC 5.

In certain embodiments, the pemetrexed is administered intravenously at a dose from 250 mg/m² to 500 mg/m².

In certain embodiments, the pemetrexed is administered intravenously at a dose about 500 mg/m².

In certain embodiments, the pemetrexed is administered intravenously after a vitamin supplementation.

In certain embodiments, the ADC is tusamitamab and the anti-PD-1 antibody is pembrolizumab.

In certain embodiments, the anti-PD-1 antibody or the anti-PD-L1 antibody and the ADC are administered about once every three weeks, the ADC being tusamitamab ravtansine and being administered to the subject intravenously in a dose of about 120 mg/m².

In certain embodiments, the anti-PD-1 antibody or the anti-PD-L1 antibody and the ADC are administered about once every three weeks, the ADC being tusamitamab ravtansine and being administered to the subject intravenously in a dose of about 150 mg/m².

In certain embodiments, the anti-PD-1 antibody is pembrolizumab.

In certain embodiments, the anti-PD-1 antibody is administered to the subject intravenously in a dose of about 200 mg.

In some embodiments, an item 1 of disclosure relates to a combination of (i) an antibody-drug conjugate (ADC) comprising an anti-CEACAM5 antibody and (ii) an anti-PD-1 antibody or anti-PD-L1 antibody, in an effective amount, for use in the treatment of a cancer in a subject in need thereof, wherein the cancer expresses CEACAM5.

In some embodiments, an item 2 of the disclosure relates to a combination according to item 1 wherein the anti-CEACAM5 antibody comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

In some embodiments, an item 3 of the disclosure relates to a combination according to item 1 or 2, wherein the anti-CEACAM5 antibody comprises a variable domain of a heavy chain (VH) consisting of SEQ ID NO: 6 and a variable domain of a light chain (VL) consisting of SEQ ID NO: 7.

In some embodiments, an item 4 of the disclosure relates to a combination according to any one of items 1 to 3, wherein the anti-CEACAM5 antibody is tusamitamab.

In some embodiments, an item 5 of the disclosure relates to a combination according to any one of items 1 to 4, wherein the ADC comprises at least one cytotoxic agent.

In some embodiments, an item 6 of the disclosure relates to a combination according to item 5, wherein the cytotoxic agent is selected from the group consisting of radioisotopes, protein toxins, small molecule toxins, and any combination thereof.

In some embodiments, an item 7 of the disclosure relates to a combination according to item 6, wherein the small molecule toxin is selected from the group consisting of antimetabolites, DNA-alkylating agents, DNA-cross-linking agents, DNA-intercalating agents, anti-microtubule agents, topoisomerase inhibitors, and any combination thereof.

In some embodiments, an item 8 of the disclosure relates to a combination according to item 7, wherein the anti-microtubule agent is selected from the group consisting of taxanes, vinca alkaloids, maytansinoids, colchicine, podophyllotoxin, griseofulvin, and any combination thereof.

In some embodiments, an item 9 of the disclosure relates to a combination according to item 8, wherein the maytansinoid is selected from the group consisting of N2′-deacetyl-N2′-(3-mercapto-1-oxopropyl)-maytansine (DM1), N2′-deacetyl-N2′(4-methyl-4-mercapto-1-oxopentyl)-maytansine (DM4), and any combination thereof.

In some embodiments, an item 10 of the disclosure relates to a combination according to any one of items 5 to 9, wherein the anti-CEACAM5 antibody is covalently attached via a cleavable or non-cleavable linker to the at least one cytotoxic agent.

In some embodiments, an item 11 of the disclosure relates to a combination according to item 10, wherein said linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

In some embodiments, an item 12 of the disclosure relates to a combination according to any one of items 1 to 11, wherein the ADC is characterized by a drug-to-antibody ratio (DAR) ranging from 1 to 10.

In some embodiments, an item 13 of the disclosure relates to a combination according to any one of items 1 to 12, wherein the ADC is tusamitamab ravtansine.

In some embodiments, an item 14 of the disclosure relates to a combination according to any one of items 1 to 13, wherein the cancer expresses CEACAM5 with moderate or high intensity defined by immunohistochemistry.

In some embodiments, an item 15 of the disclosure relates to a combination according to any one of items 1 to 14, wherein the cancer expresses CEACAM5 with moderate intensity (immunohistochemistry intensity ≥2+ in ≥1% to <50% of tumor cells).

In some embodiments, an item 16 of the disclosure relates to a combination according to any one of items 1 to 14, wherein the cancer expresses CEACAM5 with high intensity (immunohistochemistry intensity ≥2+ in ≥50% of tumor cells).

In some embodiments, an item 17 of the disclosure relates to a combination according to any one of items 1 to 16, wherein the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g cholangiacarcinoma), prostate cancer, and skin cancer.

In some embodiments, an item 18 of the disclosure relates to a combination according to item 17, wherein the cancer is gastric cancer, gastroesophageal junction cancer, or esophageal cancer.

In some embodiments, an item 19 of the disclosure relates to a combination according to item 17, wherein the cancer is lung cancer.

In some embodiments, an item 20 of the disclosure relates to a combination according to item 19, wherein the lung cancer is nonsquamous non-small cell lung cancer (NSQ NSCLC).

In some embodiments, an item 21 of the disclosure relates to a combination according to item 20, wherein the subject has advanced or metastatic NSQ NSCLC.

In some embodiments, an item 22 of the disclosure relates to a combination according to item 20, wherein the subject has NSQ NSCLC with no epidermal growth factor receptor (EGFR) sensitizing mutation or v-raf murine sarcoma viral oncogene homolog B1 (BRAF) mutation or anaplastic lymphoma kinase/c-ros oncogene 1 (ALK/ROS) alterations.

In some embodiments, an item 23 of the disclosure relates to a combination according to any one of items 1 to 22, wherein the subject has received no prior systemic chemotherapy for treatment of the cancer.

In some embodiments, an item 24 of the disclosure relates to a combination according to any one of items 1 to 23, wherein the anti-PD-1 antibody is selected from the group consisting of pembrolizumab, nivolumab, cemiplimab, sintilimab, dostarlimab, and tislelizumab.

In some embodiments, an item 25 of the disclosure relates to a combination according to item 24, wherein the anti-PD-1 antibody is pembrolizumab.

In some embodiments, an item 26 of the disclosure relates to a combination according to any one of items 1 to 23, wherein the anti-PD-L1 antibody is selected from the group consisting of atezolizumab, avelumab, and durvalumab.

In some embodiments, an item 27 of the disclosure relates to a combination according to any one of items 1 to 26, wherein the anti-PD-1 antibody or the anti-PD-L1 antibody and the ADC are administered sequentially.

In some embodiments, an item 28 of the disclosure relates to a combination according to any one of items 1 to 26, wherein the anti-PD-1 antibody or the anti-PD-L1 antibody is administered before the ADC.

In some embodiments, an item 29 of the disclosure relates to a combination according to any one of items 1 to 26, wherein the anti-PD-1 antibody or the anti-PD-L1 antibody and the ADC are administered simultaneously.

In some embodiments, an item 30 of the disclosure relates to a combination according to any one of items 1 to 29, wherein anti-PD-1 antibody or the anti-PD-L1 antibody is administered to the subject intravenously in a dose of about 200 mg to about 400 mg.

In some embodiments, an item 31 of the disclosure relates to a combination according to item 30, wherein the anti-PD-1 antibody or the anti-PD-L1 antibody is administered to the subject intravenously in a dose selected from 200 mg, 350 mg, 360 mg, and 400 mg.

In some embodiments, an item 32 of the disclosure relates to a combination according to any one of items 13 to 31, wherein the tusamitamab ravtansine is administered to the subject intravenously in a dose of about 60 mg/m² to about 190 mg/m².

In some embodiments, an item 33 of the disclosure relates to a combination according to item 32, wherein the tusamitamab ravtansine is administered to the subject intravenously in a dose of about 120 mg/m² to about 170 mg/m².

In some embodiments, an item 34 of the disclosure relates to a combination according to any one of items 30 to 33, wherein the anti-PD-1 antibody or the anti-PD-L1 antibody is administered to the subject intravenously in a dose of about 200 mg and the tusamitamab ravtansine is administered to the subject intravenously in a dose of about 120 mg/m² to about 170 mg/m².

In some embodiments, an item 35 of the disclosure relates to a combination according to item 34, wherein the anti-PD-1 antibody or the anti-PD-L1 antibody and the tusamitamab ravtansine are administered once every three weeks.

In some embodiments, an item 36 of the disclosure relates to a combination according to any one of items 30 to 33, wherein the anti-PD-1 antibody or the anti-PD-L1 antibody is administered to the subject intravenously in a dose of about 400 mg and the tusamitamab ravtansine is administered to the subject intravenously in a dose of about 120 mg/m² to about 170 mg/m².

In some embodiments, an item 37 of the disclosure relates to a combination according to item 36, wherein the anti-PD-1 antibody or the anti-PD-L1 antibody is administered once every six weeks and the tusamitamab ravtansine is administered once every three weeks.

In some embodiments, an item 38 of the disclosure relates to a combination according to any one of items 1 to 37, wherein the ADC is tusamitamab ravtansine and the anti-PD-1 antibody is pembrolizumab.

In some embodiments, an item 39 of the disclosure relates to a combination according to any one of items 1 to 36, wherein the anti-PD-1 antibody or the anti-PD-L1 antibody and the ADC are administered about once every three weeks, the ADC being tusamitamab ravtansine and being administered to the subject intravenously in a dose of about 120 mg/m².

In some embodiments, an item 40 of the disclosure relates to a combination according to any one of items 1 to 36, wherein the anti-PD-1 antibody or the anti-PD-L1 antibody and the ADC are administered about once every three weeks, the ADC being tusamitamab ravtansine and being administered to the subject intravenously in a dose of about 150 mg/m².

In some embodiments, an item 41 of the disclosure relates to a combination according to the item 39 or 40, wherein the anti-PD-1 antibody is pembrolizumab.

In some embodiments, an item 42 of the disclosure relates to a combination according to any one of items 39 to 41, wherein the anti-PD-1 antibody is administered to the subject intravenously in a dose of about 200 mg.

In some embodiments, an item 43 of the disclosure relates to a combination according to any one of items 1 to 38, further comprising administering (iii) a platinum-based chemotherapy to the subject.

In some embodiments, an item 44 of the disclosure relates to a combination according to item 43, wherein the platinum-based chemotherapy is selected from the group consisting of cisplatin and carboplatin.

In some embodiments, an item 45 of the disclosure relates to a combination according to item 43 or 44, wherein the anti-PD-1 antibody or the anti-PD-L1 antibody is administered before the ADC and the platinum-based chemotherapy.

In some embodiments, an item 46 of the disclosure relates to a combination according to any one of items 43 to 45, wherein the anti-PD-1 antibody or the anti-PD-L1 antibody, the ADC, and the platinum-based chemotherapy are administered to the subject for at least four cycles.

In some embodiments, an item 47 of the disclosure relates to a combination according to any one of items 39 to 42, wherein the platinum-based chemotherapy is cisplatin.

In some embodiments, an item 48 of the disclosure relates to a combination according to item 47, wherein the cisplatin is administered intravenously to the subject in a dose from 38 mg/m² to 75 mg/m².

In some embodiments, an item 49 of the disclosure relates to a combination according to item 48, wherein the cisplatin is administered intravenously to the subject in a dose of about 75 mg/m².

In some embodiments, an item 50 of the disclosure relates to a combination according to any one of items 43 to 46, wherein the platinum-based chemotherapy is carboplatin.

In some embodiments, an item 51 of the disclosure relates to a combination according to item 50, wherein the carboplatin is administered intravenously to the subject in a dose of about (target AUC)×[(140−age)×(weight in kg)/serum creatinine (mg/dL)×72(×0.85 if female)+25], wherein the target AUC is from AUC 2.5 to AUC 5.

In some embodiments, an item 52 of the disclosure relates to a combination according to item 51, wherein the target AUC is AUC 5.

In some embodiments, an item 53 of the disclosure relates to a combination according to any one of items 1 to 52, further comprising administering pemetrexed to the subject.

In some embodiments, an item 54 of the disclosure relates to a combination according to item 53, wherein the pemetrexed is administered intravenously at a dose from 250 mg/m² to 500 mg/m².

In some embodiments, an item 55 of the disclosure relates to a combination according to item 54, wherein the pemetrexed is administered intravenously at a dose about 500 mg/m².

In some embodiments, an item 56 of the disclosure relates to a combination according to any one of items 53 to 54, wherein the pemetrexed is administered intravenously after a vitamin supplementation.

In some embodiments, an item 57 of the disclosure relates to a combination according to any one of items 53 to 56, wherein the anti-PD-1 antibody or the anti-PD-L1 antibody and the ADC are administered about once every three weeks, the ADC being tusamitamab ravtansine and being administered to the subject intravenously in a dose of about 120 mg/m².

In some embodiments, an item 58 of the disclosure relates to a combination according to any one of items 53 to 56, wherein the anti-PD-1 antibody or the anti-PD-L1 antibody and the ADC are administered about once every three weeks, the ADC being tusamitamab ravtansine and being administered to the subject intravenously in a dose of about 150 mg/m².

In some embodiments, an item 59 of the disclosure relates to a combination according to the item 57 or 58, wherein the anti-PD-1 antibody is pembrolizumab.

In some embodiments, an item 60 of the disclosure relates to a combination according to any one of items 57 to 59, wherein the anti-PD-1 antibody is administered to the subject intravenously in a dose of about 200 mg.

### BRIEF DESCRIPTION OF THE DRAWINGS

FIG. 1 is a schematic diagram depicting the decision process for tusamitamab ravtansine dose in Part A, Part B, and Part C of a Phase 2 clinical trial according to embodiments of the present disclosure.

FIG. 2 is a schematic diagram depicting the study design of Part A of a Phase 2 clinical trial according to embodiments of the present disclosure.

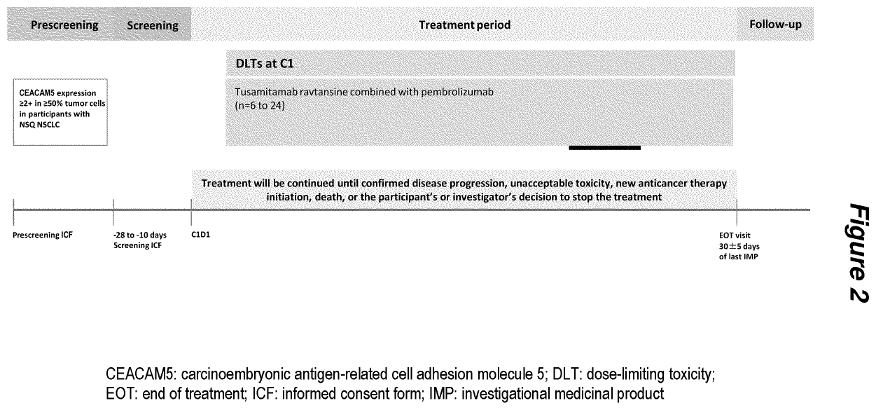

FIG. 3 is a schematic diagram depicting the study design of Part B of a Phase 2 clinical trial according to embodiments of the present disclosure.

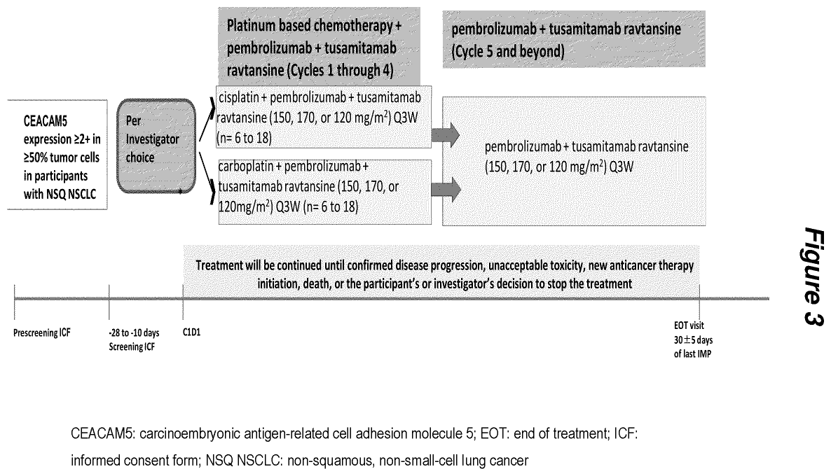

FIG. 4 is a schematic diagram depicting the study design of Part C of a Phase 2 clinical trial according to embodiments of the present disclosure.

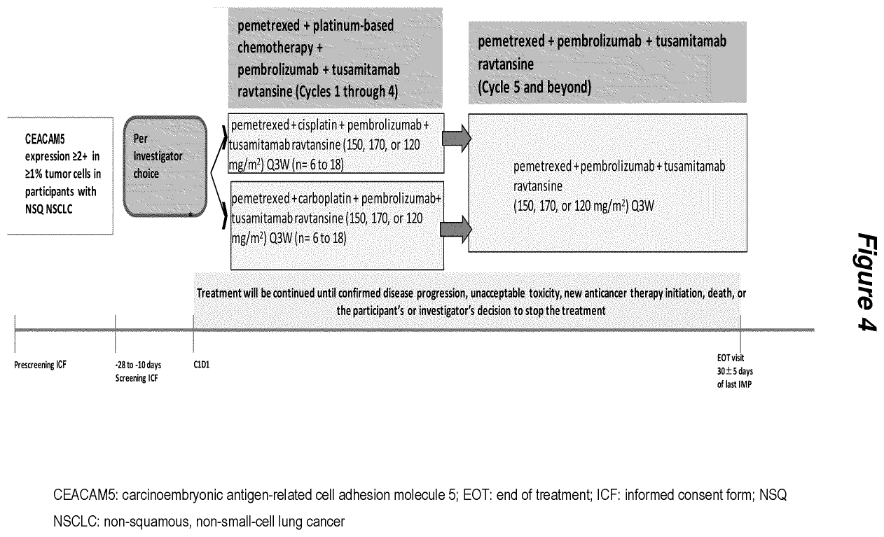

FIGS. 5A and B show the activity of the immunoconjugate huMAb2-3-SPDB-DM4 and the anti-muPD-1 antibody, as single agents or in combination against subcutaneous colon MC38 syngeneic tumor model in C57BI/6 mice. Tumor volume evolution by treatment group. The curves represent medians + or − Maximum Administered Dose (MAD) at each day for each group.

FIGS. 6A and B show the activity of the immunoconjugate huMAb2-3-SPDB-DM4 and the anti-mu/huPD-L1 antibody, as single agents or in combination against subcutaneous colon MC38 syngeneic tumor model in C57BI/6 mice. Tumor volume evolution by treatment group. The curves represent medians + or − MAD at each day for each group.

### DETAILED DESCRIPTION OF THE DISCLOSURE

The disclosure provides pharmaceutical compositions and methods of using these compositions for the treatment of cancer (e.g., lung cancer, including NSQ NSCLC), and the improvement of at least one symptom of the disease. These compositions include at least one antibody that specifically binds (CEACAM5).

Tusamitamab ravtansine is an immunoconjugate ADC combining a humanized anti-CEACAM5 antibody (tusamitamab) and the maytansinoid derivative 4 (DM4) [N2′-deacetyl-N2′-(4-methyl-4-mercapto-1-oxopentyl)-maytansine], a potent antimitotic agent that inhibits microtubule assembly. DM4 is covalently bound to the antibody through an optimized linker SPDB [N-succinimidyl 4-(2-pyridyldithio)-butyrate] that is stable in plasma and cleavable inside cells. After binding and internalization in targeted cancer cells, the ADC is degraded, releasing cytotoxic DM4 metabolites.

Tusamitamab ravtansine specifically binds to the A3B3 domain of human CEACAM5 and does not recognize other CEACAMs presenting A or/and B domains in their structure (CEACAM1, CEACAM6, CEACAM7 and CEACAM8). The naked antibody and the ADC bind to recombinant human CEACAM5 with an affinity of ˜0.02 nM (ELISA) and display high affinity for CEACAM5 expressing tumor cells (K_{D}^{APP} 0.24-0.68 nM).

After binding to the CEACAM5 antigen, tusamitamab ravtansine is internalized by the cancer cells via antigen-mediated endocytosis, delivered to lysosomes and degraded into the lysine-linked derivative lysine-SPDB-DM4. The lysine-SPDB-DM4 gets further degraded in DM4 that is subsequently S-methylated to form methyl-DM4 [Me-DM4]; all three metabolites have potent cytotoxic activity through binding to tubulin and inhibition of microtubule polymerization.

As used herein, CEACAM5 expressing cancer refers to several types of cancer, including colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiacarcinoma), prostate cancer, and skin can. In some embodiments, the cancer is lung cancer. In some embodiments, the lung cancer is non-squamous non-small cell lung cancer.

In certain embodiments, the cancer is a moderate CEACAM5 expresser. Moderate CEACAM5 expressers have ≥2+ intensity in ≥1% to <50% of expressing tumor cell population, as measured using immunohistochemistry.

In certain embodiments, the cancer is a high CEACAM5 expresser. High CEACAM5 expressers have ≥2+ intensity in ≥50% of expressing tumor cell population, as measured using immunohistochemistry. High CEACAM5 expressers represent ˜20% of lung cancer.

The ADC in monotherapy was analyzed in a Phase 1/2 study in heavily pre-treated high CEACAM5 expressers. The ADC demonstrated competitive Overall Response Rate (ORR) and Duration of Response (DoR). Most common Adverse Drug Reactions (ADRs) were ocular toxicity (reversible without treatment discontinuation), and minimal hematological/nerve toxicity.

### Non-Small Cell Lung Cancer

Non-small cell lung cancer is a disease in which malignant (cancer) cells form in the tissues of the lung. Smoking is the major cause of the disease. This is a type of epithelial lung cancer other than small cell lung carcinoma. There are several types of non-small cell lung cancer. Each type of non-small cell lung cancer has different kinds of cancer cells. The cancer cells of each type grow and spread in different ways. The types of non-small cell lung cancer are named for the kinds of cells found in the cancer and how the cells look under a microscope: (1) squamous cell carcinoma: Cancer that begins in squamous cells, which are thin, flat cells that look like fish scales. This is also called epidermoid carcinoma. (2) large cell carcinoma: Cancer that may begin in several types of large cells. (3) adenocarcinoma: Cancer that begins in the cells that line the alveoli and make substances such as mucus.

Selectively targeting potent cytotoxics to tumor cells using antibody drug conjugates (ADCs) has now been shown to be an effective strategy for the treatment of cancer, as demonstrated by the recent approvals of brentuximab vedotin for the treatment of Hodgkin lymphoma and trastuzumab emtansine (T-DM1) for the treatment of relapsed metastatic HER2+ breast cancer (Young A. et al., 2012 J Clin Oncol. 30 (18): 2183-9; Verma, S. et al., 2012 N Engl J Med. 19:1783-91). Many other malignant diseases with unmet medical needs could benefit from such therapeutic options. The mechanism of action of ADCs begins with its binding to a specific antigen, sufficiently expressed on the tumor cells in order to achieve a selective and efficient internalization of the drug.

Radical surgery (e.g., pneumonectomy, lobectomy, segmentectomy or wedge resection, sleeve resection) is the standard of care for fit stage I NSCLC patients. Adjuvant treatment should be offered only as part of an investigation trial. Stage II and IIIA adjuvant cisplatin-based chemotherapy remains the gold standard for completely resected NSCLC tumors. Other chemotherapeutic agents used in combination with cisplatin, or with each other, may include carboplatin, paclitaxel (Taxol), albumin-bound paclitaxel (nab-paclitaxel, Abraxane), docetaxel (Taxotere), Gemcitabine (Gemzar), vinorelbine (Navelbine), irinotecan (Camptosar), etoposide (VP-16), vinblastine, and pemetrexed (Alimta). Additionally, radiotherapy may be used in patients with N2 lymph nodes. In advanced stage IIIB/IV or inoperable NSCLC pts, treatment may include multiple cycles of cisplatin-based chemotherapy plus a 3rd generation cytotoxic agent or a cytostatic drug (anti-EGFR, anti-VEGFR). (See Zarogoulidis et al., J Thorac Dis. 2013 September; 5 (Suppl 4): S389-S396.)

Treatments for cancers, including lung cancers, can include angiogenesis inhibitors, epidermal growth factor receptor (EGFR) inhibitors, and immune checkpoint inhibitors.

Angiogenesis inhibitors may include, but are not limited to, Axitinib (Inlyta), Bevacizumab (Avastin), Cabozantinib (Cometriq), Everolimus (Afinitor, Zortress), Lenalidomide (Revlimid), Pazopanib (Votrient), Ramucirumab (Cyramza), Regorafenib (Stivarga), Sorafenib (Nexavar), Sunitinib (Sutent), Thalidomide (Synovir, Thalomid), Vandetanib (Caprelsa), and Ziv-aflibercept (Zaltrap).

EGFR inhibitors may include, but are not limited to, gefitinib (Iressa), erlotinib (Tarceva), lapatinib (Tykerb), cetuximab (Erbitux), neratinib (Nerlynx), osimertinib (Tagrisso), panitumumab (Vectibix), vandetanib (Caprelsa), necitumumab (Protrazza), and dacomitinib (Vizimpro).

Immune checkpoint inhibitors may include, but are not limited to, Programmed Death 1 receptor (PD-1) binding agents (e.g., pembrolizumab, nivolumab, cemiplimab, sintilimab, dostarlimab, and tislelizumab), Programmed Death-ligand 1 (PD-L1) binding agents (e.g., atezolizumab, avelumab, durvalumab), CTLA-4 binding agents (e.g., ipilimumab), OX40 or OX40L binding agents, Adenosine A2A receptor binding agents, B7-H3 binding agents, B7-H4 binding agents, BTLA binding agents, Indoleamine 2,3-dioxygenase binding agents, Killer-cell Immunoglobulin-like Receptor (KIR) binding agents, Lymphocyte Activation Gene-3 (LAG-3) binding agents, nicotinamide adenine dinucleotide phosphate NADPH oxidase isoform (NOX2) binding agents, T-cell Immunoglobulin domain and Mucin domain 3 (TIM-3) binding agents, V-domain Ig suppressor of T cell activation (VISTA) binding agents, Glucocorticoid-Induced TNFR family Related gene (GITR) binding agents, and Sialic acid-binding immunoglobulin-type lectin 7 (SIGLEC7) binding agents.

### CEACAM5 and Indications:

Carcinoembryonic antigen (CEA) is a glycoprotein involved in cell adhesion. CEA was first identified in 1965 (Gold and Freedman, J Exp Med, 121, 439, 1965) as a protein normally expressed by fetal gut during the first six months of gestation, and found in cancers of the pancreas, liver and colon. The CEA family belongs to the immunoglobulin superfamily. The CEA family, which consists of 18 genes, is sub-divided in two sub-groups of proteins: the carcinoembryonic antigen-related cell adhesion molecule (CEACAM) sub-group and the pregnancy-specific glycoprotein subgroup (Kammerer & Zimmermann, BMC Biology 2010, 8:12).

In humans, the CEACAM sub-group consists of 7 members: CEACAM1, CEACAM3, CEACAM4, CEACAM5, CEACAM6, CEACAM7, CEACAM8. Numerous studies have shown that CEACAM5, identical to the originally identified CEA, is highly expressed on the surface of colorectal, gastric, gastroesophageal junction, esophageal, lung, breast, prostate, ovary, cervix and bladder tumor cells and weakly expressed in few normal epithelial tissues such as columnar epithelial and goblet cells in colon, mucous neck cells in the stomach, and squamous epithelial cells in esophagus and cervix (Hammarström et al, 2002, in “Tumor Markers, Physiology, Pathobiology, Technology and Clinical Applications” Eds. Diamandis E. P. et al., AACC Press, Washington pp 375). Thus, CEACAM5 may constitute a therapeutic target suitable for tumor-specific targeting approaches, such as immunoconjugates.

The extracellular domains of CEACAM family members are composed of repeated immunoglobulin-like (Ig-like) domains which have been categorized in 3 types, A, B and N, according to sequence homologies. CEACAM5 contains seven such domains, namely N, A1, B1, A2, B2, A3, and B3.

CEACAM5 A1, A2, and A3 domains, on one hand, and B1, B2, and B3 domains, on the other hand, show high sequence homologies, the A domains of human CEACAM5 presenting from 84 to 87% pairwise sequence similarity, and the B domains from 69 to 80%. Furthermore, other human CEACAM members presenting A and/or B domains in their structure, namely CEACAM1, CEACAM6, CEACAM7, and CEACAM8, show homology with human CEACAM5. In particular, the A and B domains of human CEACAM6 protein display sequence homologies with A1 and A3 domains, and any of B1 to B3 domains of human CEACAM5, respectively, which are even higher than observed among the A domains and the B domains of human CEACAM5.

An embodiment of the disclosure is a method of treating cancer, wherein the cancer expresses CEACAM5.

In an embodiment the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g cholangiacarcinoma), prostate cancer, and skin cancer.

In an embodiment the cancer is gastric cancer, gastroesophageal junction cancer or esophageal cancer.

In an embodiment the cancer is lung cancer.

In an embodiment the cancer is non-squamous non-small cell lung cancer (NSQ-NSCLC).

### Anti-CEACAM5 Antibody:

Numerous anti-CEA antibodies were generated in view of CEA-targeted diagnostic or therapeutic purposes. Specificity towards related antigens has always been mentioned as a concern in this field, as an example by Sharkey et al (1990, Cancer Research 50, 2823). Due to the above-mentioned homologies, some of previously described antibodies may demonstrate binding to repetitive epitopes of CEACAM5 present in the different immunoglobulin domains show cross-reactivity to other CEACAM members such as CEACAM1, CEACAM6, CEACAM7, or CEACAM8, lacking specificity to CEACAM5. The specificity of the anti-CEACAM5 antibody is desired in view of CEA-targeted therapies such that it binds to human CEACAM5-expressing tumor cells but does not bind to some normal tissues expressing other CEACAM members. It is noteworthy that CEACAM1, CEACAM6, and CEACAM8 have been described as expressed by neutrophils of human and non-human primates (Ebrahimmnejad et al, 2000, Exp Cell Res, 260, 365; Zhao et al, 2004, J Immunol Methods 293, 207; Strickland et al, 2009 J Pathol, 218, 380) where they have been shown to regulate granulopoiesis and to play a role in immune response.

The ADC tusamitamab ravtansine has been shown to be capable of being internalized into cells expressing CEACAM5 after binding, and to induce cytotoxic activity on tumor cells in vitro. Tusamitamab ravtansine is also able to markedly inhibit tumor growth in vivo in mice bearing human primary colon and stomach tumors. See WO 2014/079886, which is incorporated herein by reference in its entirety.

As used herein, the term “about” in quantitative terms refers to plus or minus 10% of the value it modifies (rounded up to the nearest whole number if the value is not sub-dividable, such as a number of molecules or nucleotides). For example, the phrase “about 100 mg” would encompass 90 mg to 110 mg, inclusive; the phrase “about 2500 mg” would encompass 2250 mg to 2750 mg. When applied to a percentage, the term “about” refers to plus or minus 10% relative to that percentage. For example, the phrase “about 20%” would encompass 18-22% and “about 80%” would encompass 72-88%, inclusive. Moreover, where “about” is used herein in conjunction with a quantitative term it is understood that in addition to the value plus or minus 10%, the exact value of the quantitative term is also contemplated and described. For example, the term “about 23%” expressly contemplates, describes, and includes exactly 23%.

It is to be noted that the term “a” or “an” entity refers to one or more of that entity; for example, “a symptom,” is understood to represent one or more symptoms. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein “CEACAM5” designates the “carcinoembryonic antigen-related cell adhesion molecule 5”, also known as “CD66e” (Cluster of Differentiation 66e) or CEA. CEACAM5 is a glycoprotein involved in cell adhesion. CEACAM5 is highly expressed in particular on the surface of colorectal, gastric, gastroesophageal junction, esophageal, lung, and uterine tumor cells.

A reference sequence of full length human CEACAM5, including signal peptide (positions 1-34) and propeptide (positions 686-702), is available from the GenBank database under accession number AAA51967.1. Five nonsynonymous SNPs have been identified with a frequency higher than 2% in Caucasian population, four of them being localised in the N domain (at positions 80, 83, 112, 113), the last one in the A2 domain (at position 398) of human CEACAM5.

It is understood that wherever aspects or embodiments are described herein with the language “comprising,” otherwise analogous aspects described in terms of “consisting of” and/or “consisting essentially of” are also provided.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide, or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, VHH or NANOBODY® (e.g., monovalent VHH, and bivalent VHH), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may in various embodiments consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may in various embodiments comprise a homo-dimer or hetero-dimer (or other multimer) of any of the variable and constant domain configurations listed above in non-covalent association with one another and/or with one or more monomeric VH or VL domain (e.g., by disulfide bond(s)).

In specific embodiments, the antibody or antibody fragment for use in the method of the disclosure may be a multispecific antibody, which may be specific for different epitopes of one target polypeptide or may contain antigen-binding domains specific for epitopes of more than one target polypeptide. An exemplary bi-specific antibody format that can be used in the context of the present disclosure involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise an Y96F modification (by IMGT; Y436F by EU). Further modifications that may be found within the second CH3 include: D16E, L18M, N44S, K52N, V57M, and V821 (by IMGT; D356E, L358M, N384S, K392N, V397M, and V422I by EU) in the case of IgG1 antibodies; N44S, K52N, and V821 (IMGT; N384S, K392N, and V422I by EU) in the case of IgG2 antibodies; and Q15R, N44S, K52N, V57M, R69K, E79Q, and V821 (by IMGT; Q355R, N384S, K392N, V397M, R409K, E419Q, and V422I by EU) in the case of IgG4 antibodies.

Variations on the bi-specific antibody format described above are contemplated within the scope of the present disclosure. Any multispecific antibody format, including the exemplary bispecific antibody formats disclosed herein, may in various embodiments be adapted for use in the context of an antigen-binding fragment of an anti-CEACAM5 antibody using routine techniques available in the art.

The CEACAM5 antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are back-mutated to the corresponding germline residue(s) or to a conservative amino acid substitution (natural or non-natural) of the corresponding germline residue(s) (such sequence changes are referred to herein as “germline back-mutations”). A person of ordinary skill in the art, starting with the heavy and light chain variable region sequences disclosed herein, can easily produce numerous antibodies and antigen-binding fragments which comprise one or more individual germline back-mutations or combinations thereof. In certain embodiments, all of the framework residues and/or CDR residues within the VH and/or VL domains are mutated back to the germline sequence. In other embodiments, only certain residues are mutated back to the germline sequence, e.g., only the mutated residues found within the first 8 amino acids of FR1 or within the last 8 amino acids of FR4, or only the mutated residues found within CDR1, CDR2 or CDR3. Furthermore, the antibodies of the present disclosure may contain any combination of two or more germline back-mutations within the framework and/or CDR regions, i.e., wherein certain individual residues are mutated back to the germline sequence while certain other residues that differ from the germline sequence are maintained. Once obtained, antibodies and antigen-binding fragments that contain one or more germline back-mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies featured in the disclosure may in various embodiments nonetheless include amino acid residues not encoded by human germline immunoglobulin sequences (e.g., mutations introduced by random or site-specific mutagenesis in vitro or by somatic mutation in vivo), for example in the CDRs and in some embodiments CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., (1992) Nucl. Acids Res. 20:6287-6295, incorporated herein by reference in its entirety,) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In an embodiment, an immunoglobulin molecule comprises a stable four chain construct of approximately 150-160 kDa in which the dimers are held together by an interchain heavy chain disulfide bond. In another embodiment, the dimers are not linked via inter-chain disulfide bonds and a molecule of about 75-80 kDa is formed composed of a covalently coupled light and heavy chain (half-antibody). These embodiments/forms have been extremely difficult to separate, even after affinity purification.

The term “humanised antibody” or “humanized antibody” refers to an antibody which is wholly or partially of non-human origin and which has been modified to replace certain amino acids, for instance in the framework regions of the VH and VL domains, in order to avoid or minimize an immune response in humans. The constant domains of a humanized antibody are most of the time human CH and CL domains.

Numerous methods for humanisation/humanization of an antibody sequence are known in the art; see e.g. the review by Almagro & Fransson (2008) Front Biosci. 13:1619-1633. One commonly used method is CDR grafting, or antibody reshaping, which involves grafting of the CDR sequences of a donor antibody, generally a mouse antibody, into the framework scaffold of a human antibody of different specificity. Since CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing, in which non-surface exposed residues of non-human origin are retained, while surface residues are altered to human residues. Another alternative technique is known as “guided selection” (Jespers et al. (1994) Biotechnology 12, 899) and can be used to derive from a murine antibody a fully human antibody conserving the epitope and binding characteristics of the parental antibody.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al., (1993) Molecular Immunology 30:105, incorporated by reference in its entirety) to levels typically observed using a human IgG1 hinge. The instant disclosure encompasses in various embodiments antibodies having one or more mutations in the hinge, CH2 or CH3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” In various embodiments, the isolated antibody also includes an antibody in situ within a recombinant cell. In other embodiments, isolated antibodies are antibodies that have been subjected to at least one purification or isolation step. In various embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” CEACAM5, as used herein, includes antibodies that bind CEACAM5 or portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay. Specific binding can also be characterized by a dissociation constant of at least about 1×10⁻⁶ M or smaller. In other embodiments, the dissociation constant is at least about 1×10⁻⁷ M, 1×10⁻⁸ M, or 1×10⁻⁹ M. An isolated antibody that specifically binds human CEACAM5 may, however, have cross-reactivity to other antigens, such as CEACAM5 molecules from other (non-human) species.

The term “surface plasmon resonance”, as used herein, refers to an optical phenomenon that allows for the analysis of real-time interactions by detection of alterations in protein concentrations within a biosensor matrix, for example using the BIACORE™ system (Biacore Life Sciences division of GE Healthcare, Piscataway, NJ).

The term “KD”, as used herein, is intended to refer to the equilibrium dissociation constant of an antibody-antigen interaction.

“Affinity” is defined, in theory, by the equilibrium association between the whole antibody and the antigen. It can be experimentally assessed by a variety of known methods, such as measuring association and dissociation rates with surface plasmon resonance or measuring the EC50 (or apparent KD) in an immunochemical assay (ELISA, FACS). In these assays, the EC50 is the concentration of the antibody which induces a response halfway between the baseline and maximum after some specified exposure time on a defined concentration of antigen by ELISA (enzyme-linked immuno-sorbent assay) or cell expressing the antigen by FACS (Fluorescence Activated Cell Sorting).

A monoclonal antibody binding to antigen 1 (Ag1) is “cross-reactive” to antigen 2 (Ag2) when the EC50s are in a similar range for both antigens. In the present application, a monoclonal antibody binding to Ag1 is cross-reactive to Ag2 when the ratio of affinity of Ag2 to affinity of Ag1 is equal or less than 10 (for instance 5, 2, 1 or 0.5), affinities being measured with the same method for both antigens.

Affinity for human CEACAM5 or for Macaca fascicularis CEACAM5 may be determined as the EC50 value in an ELISA using soluble recombinant CEACAM5 as capture antigen.

The antibody of the disclosure may also have an apparent dissociation constant (apparent KD), as may be determined by FACS analysis on tumor cell line MKN45 (DSMZ, ACC 409) or on xenograft tumor cells deriving from patient (CR-IGR-034P available from Oncodesign Biotechnology, tumor collection CREMEC), which is ≤25 nM, for instance ≤20 nM, ≤10 nM, ≤5 nM, ≤3 nM or ≤1 nM. The apparent KD may be within the range 0.01-20 nM, or may be within the range 0.1-20 nM, 0.1-10 nM, or 0.1-5 nM.

Additionally, antibodies according to the disclosure have been shown to be able to detect CEACAM5 expression by immunohistochemistry in frozen and formalin-fixed and paraffin embedded (FFPE) tissue sections.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The anti-CEACAM5 antibodies useful for the methods described herein may in various embodiments include one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes in various embodiments methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). Numerous antibodies and antigen-binding fragments may be constructed which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a certain germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present disclosure.

The present disclosure also includes methods involving the use of anti-CEACAM5 antibodies comprising variants of any of the HCVR, LCVR, and/or CDR amino acid sequences disclosed herein having one or more conservative substitutions. For example, the present disclosure includes the use of anti-CEACAM5 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

According to the present disclosure, the anti-CEACAM5 antibody, or antigen-binding fragment thereof, in various embodiments comprises a heavy chain variable region (HCVR), light chain variable region (LCVR), and/or complementarity determining regions (CDRs) comprising any of the amino acid sequences of the anti-CEACAM5 antibodies described in Intl. Patent Pub. No. WO 2014/079886 A1, incorporated herein by reference in its entirety.

Amino acid sequence modification(s) of the antibodies described herein are contemplated. For example, it may be desirable to improve the binding affinity and/or other biological properties of the antibody. It is known that when a humanised antibody is produced by simply grafting only CDRs in VH and VL of an antibody derived from a non-human animal in FRs of the VH and VL of a human antibody, the antigen binding activity may be reduced in comparison with that of the original antibody derived from a non-human animal. It is considered that several amino acid residues of the VH and VL of the non-human antibody, not only in CDRs but also in FRs, may be directly or indirectly associated with the antigen binding activity. Hence, substitution of these amino acid residues with different amino acid residues derived from FRs of the VH and VL of the human antibody would reduce the binding activity. In order to solve the problem, in human antibodies grafted with non-human CDRs, attempts have to be made to identify, among amino acid sequences of the FR of the VH and VL of human antibodies, an amino acid residue which is directly associated with binding of the antibody, or which interacts with an amino acid residue of a CDR, or which maintains the three-dimensional structure of the antibody and which is directly associated with binding to the antigen. The reduced antigen binding activity could be increased by replacing the identified amino acids with amino acid residues of the original antibody derived from a non-human animal.

Modifications and changes may be made in the structure of the antibodies of the present disclosure, and in the DNA sequences encoding them, and still result in a functional antibody or polypeptide with desirable characteristics.

A further object of the present disclosure also encompasses function-conservative variants of the polypeptides of the present disclosure. For example, certain amino acids may be substituted by other amino acids in a protein structure without appreciable loss of activity. Since the interactive capacity and nature of a protein define its biological functional activity, certain amino acid substitutions can be made in a protein sequence, and of course in its DNA encoding sequence, while nevertheless obtaining a protein with like properties. It is thus contemplated that various changes may be made in the antibody sequences of the disclosure, or corresponding DNA sequences which encode said polypeptides, without appreciable loss of their biological activity. It is known in the art that certain amino acids may be substituted by other amino acids having a similar hydropathic index or score and still result in a protein with similar biological activity, i.e. still obtain a biological functionally equivalent protein. It is also possible to use well-established technologies, such as alanine-scanning approaches, to identify, in an antibody or polypeptide of the disclosure, all the amino acids that can be substituted without significant loss of binding to the antigen. Such residues can be qualified as neutral, since they are not involved in antigen binding or in maintaining the structure of the antibody. One or more of these neutral positions can be substituted by alanine or by another amino acid can without changing the main characteristics of the antibody or polypeptide of the disclosure.

Neutral positions can be seen as positions where any amino acid substitution could be incorporated to the antibodies. Indeed, in the principle of alanine-scanning, alanine is chosen since it this residue does not carry specific structural or chemical features. It is generally admitted that if an alanine can be substituted for a specific amino acid without changing the properties of a protein, many other, if not all amino acid substitutions are likely to be also neutral. In the opposite case where alanine is the wild-type amino acid, if a specific substitution can be shown as neutral, it is likely that other substitutions would also be neutral. As outlined above, amino acid substitutions are generally therefore based on the relative similarity of the amino acid side-chain substituents, for example, their hydrophobicity, hydrophilicity, charge, size, and the like. Exemplary substitutions which take any of the foregoing characteristics into consideration are well known to those of skill in the art and include: arginine and lysine; glutamate and aspartate; serine and threonine; glutamine and asparagine; and valine, leucine and isoleucine.

It may be also desirable to modify the antibody of the disclosure with respect to effector function, e.g. so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing inter-chain disulfide bond formation in this region. The homodimeric antibody thus generated may have improved internalization capability and/or increased complement-mediated cell killing and/or antibody-dependent cellular cytotoxicity (ADCC) (Caron P C. et al. 1992; and Shopes B. 1992).

Another type of amino acid modification of the antibody of the disclosure may be useful for altering the original glycosylation pattern of the antibody, i.e. by deleting one or more carbohydrate moieties found in the antibody, and/or adding one or more glycosylation sites that are not present in the antibody. The presence of either of the tripeptide sequences asparagine-X-serine, and asparagine-X-threonine, where X is any amino acid except proline, creates a potential glycosylation site. Addition or deletion of glycosylation sites to the antibody is conveniently accomplished by altering the amino acid sequence such that it contains one or more of the above-described tripeptide sequences (for N-linked glycosylation sites).

Another type of modification involves the removal of sequences identified, either in silico or experimentally, as potentially resulting in degradation products or heterogeneity of antibody preparations. As examples, deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in an antibody or polypeptide of the disclosure, it may therefore be desirable to remove the site, typically by conservative substitution to remove one of the implicated residues. Such substitutions in a sequence to remove one or more of the implicated residues are also intended to be encompassed by the present disclosure.

Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. For example, such methods are described in WO87/05330.

Removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Another type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337.

In an embodiment, the anti-CEACAM5 antibody is Tusamitamab (CAS Registry No. 2349294-95-5).

Tusamitamab comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

HCDR1
(SEQ ID NO: 1)
GFVFSSYD
HCDR2
(SEQ ID NO: 2)
ISSGGGIT
HCDR3
(SEQ ID NO: 3)
AAHYFGSSGPFAY
LCDR1
(SEQ ID NO: 4)
ENIFSY
LCDR2
NTR
LCDR3
(SEQ ID NO: 5)
QHHYGTPFT

Tusamitamab comprises a variable domain of a heavy chain (VH) consisting of SEQ ID NO: 6 and a variable domain of a light chain (VL) consisting of SEQ ID NO: 7.

(SEQ ID NO: 6)
EVQLQESGPGLVKPGGSLSLSCAASGFVFSSYDMSWVRQTPERGL
EWVAYISSGGGITYAPSTVKGRFTVSRDNAKNTLYLQMNSLTSED
TAVYYCAAHYFGSSGPFAYVVGQGTLVTVSS
(SEQ ID NO: 7)
DIQMTQSPASLSASVGDRVTITCRASENIFSYLAWYQQKPGKSPK
LLVYNTRTLAEGVPSRFSGSGSGTDFSLTISSLQPEDFATYYCQH
HYGTPFTFGSGTKLEI

### Cytotoxic Payload and Immunoconjugate

The present disclosure also includes cytotoxic conjugates, or immunoconjugates, or antibody-drug conjugates, or conjugates. As used herein, all these terms have the same meaning and are interchangeable.

Accordingly, the disclosure relates to “immunoconjugates” comprising an antibody of the disclosure (e.g. anti-CEACAM5 antibody) linked or conjugated to at least one growth inhibitory agent, such as a cytotoxic agent or a radioactive isotope.

A “growth inhibitory agent”, or “anti-proliferative agent”, which can be used indifferently, refers to a compound or composition which inhibits growth of a cell, especially tumour cell, either in vitro or in vivo.

The term “cytotoxic agent” as used herein refers to a substance that inhibits or prevents the function of cells and/or causes destruction of cells. The term “cytotoxic agent” is intended to include chemotherapeutic agents, enzymes, antibiotics, and toxins such as small molecule toxins or enzymatically active toxins of bacterial, fungal, plant or animal origin, including fragments and/or variants thereof, and the various antitumor or anticancer agents disclosed below. In some embodiments, the cytotoxic agent is a taxoid, vincas, a maytansinoid or maytansinoid analog such as DM1 or DM4, a small drug, a tomaymycin or pyrrolobenzodiazepine derivative, a cryptophycin derivative, a leptomycin derivative, an auristatin or dolastatin analog, a prodrug, topoisomerase II inhibitors, a DNA alkylating agent, an anti-tubulin agent, a CC-1065 or CC-1065 analog.

As used herein “maytansinoids” denotes maytansinoids and maytansinoid analogs. Maytansinoids are drugs that inhibit microtubule formation and that are highly toxic to mammalian cells.

Examples of suitable maytansinoids include maytansinol and maytansinol analogs.

Examples of suitable maytansinol analogues include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Specific examples of suitable analogues of maytansinol having a modified aromatic ring include:
(1) C-19-dechloro (U.S. Pat. No. 4,256,746) (prepared by LAH reduction of ansamytocin P2);
(2) C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and
(3) C-20-demethoxy, C-20-acyloxy (—OCOR), +/−dechloro (U.S. Pat. No. 4,294,757) (prepared by acylation using acyl chlorides).

Specific examples of suitable analogues of maytansinol having modifications of other positions include:
(1) C-9-SH (U.S. Pat. No. 4,424,219) (prepared by the reaction of maytansinol with H2S or P2S5);
(2) C-14-alkoxymethyl (demethoxy/CH₂OR) (U.S. Pat. No. 4,331,598);
(3) C-14-hydroxymethyl or acyloxymethyl (CH₂OH or CH₂OAc) (U.S. Pat. No. 4,450,254) (prepared from Nocardia);
(4) C-15-hydroxy/acyloxy (U.S. Pat. No. 4,364,866) (prepared by the conversion of maytansinol by Streptomyces);
(5) C-15-methoxy (U.S. Pat. Nos. 4,313,946 and 4,315,929) (isolated from Trewia nudiflora);
(6) C-18-N-demethyl (U.S. Pat. Nos. 4,362,663 and 4,322,348) (prepared by the demethylation of maytansinol by Streptomyces); and
(7) 4,5-deoxy (U.S. Pat. No. 4,371,533) (prepared by the titanium trichloride/LAH reduction of maytansinol).

In an embodiment of the disclosure, the cytotoxic conjugates of the present disclosure utilize the thiol-containing maytansinoid (DM1), formally termed N2′-deacetyl-N2′-(3-mercapto-1-oxopropyl)-maytansine, as the cytotoxic agent. DM1 is represented by the following structural formula (I):

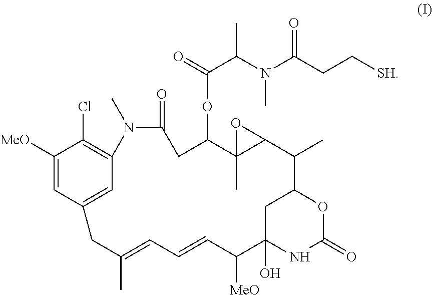 [SMILES: COc1cc2cc(c1Cl)N(C)C(=O)CC(OC(=O)C(C)N(C)C(=O)CCS)C1(C)OC1C(C)C1CC(O)(NC(=O)O1)C(OC)/C=C/C=C(\C)C2]

In another embodiment, the cytotoxic conjugates of the present disclosure utilize the thiol-containing maytansinoid DM4, formally termed N2′-deacetyl-N-2′(4-methyl-4-mercapto-1-oxopentyl)-maytansine, as the cytotoxic agent. DM4 is represented by the following structural formula (II):

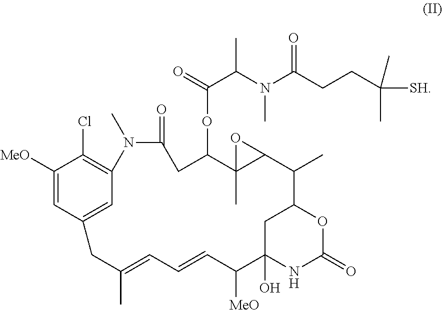 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

In further embodiments of the disclosure, other maytansines, including thiol and disulfide-containing maytansinoids bearing a mono or di-alkyl substitution on the carbon atom bearing the sulfur atom, may be used. These include a maytansinoid having, at C-3, C-14 hydroxymethyl, C-15 hydroxy, or C-20 desmethyl, an acylated amino acid side chain with an acyl group bearing a hindered sulfhydryl group, wherein the carbon atom of the acyl group bearing the thiol functionality has one or two substituents, said substituents being CH3, C2H5, linear or branched alkyl or alkenyl having from 1 to 10 reagents and any aggregate which may be present in the solution.

Examples of these cytotoxic agents and of methods of conjugation are further given in the application WO 2008/010101 which is incorporated by reference.

The term “radioactive isotope” is intended to include radioactive isotopes suitable for treating cancer, such as At211, Bi212, Er169, I131, I125, Y90, In111, P32, Re186, Re188, Sm153, Sr89, and radioactive isotopes of Lu. Such radioisotopes generally emit mainly beta-radiation. In an embodiment the radioactive isotope is alpha-emitter isotope, more precisely Thorium 227 which emits alpha-radiation.

The immunoconjugates according to the present disclosure can be prepared as described in the application WO 2004/091668, the entire content of which is incorporated herein by reference.

In some embodiments, the antibodies of the present disclosure are covalently attached, directly or via a cleavable or non-cleavable linker, to at least one cytotoxic agent or growth inhibitory agent.

“Linker”, as used herein, means a chemical moiety comprising a covalent bond or a chain of atoms that covalently attaches a polypeptide (e.g., an antibody) to a drug (or prodrug) moiety. Suitable linkers are well known in the art and include disulfide groups, thioether groups, acid labile groups, photolabile groups, peptidase labile groups and esterase labile groups. Exemplary linkers include, but are not limited to, heterobifunctional crosslinking reagents such as N-succinimidyl pyridyldithiobutyrate (SPDB), butanoic acid 4-[(5-nitro-2-pyridinyl) dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl(N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to the antibody (WO 94/11026).

The linker may be a “cleavable linker” facilitating release of the cytotoxic agent or growth inhibitory agent in the cell. For example, an acid-labile linker, a peptidase-sensitive linker, an esterase labile linker, a photolabile linker or a disulfide-containing linker (See e.g. U.S. Pat. No. 5,208,020) may be used. The linker may be also a “non-cleavable linker” (for example SMCC linker) that might lead to better tolerance in some cases.

Alternatively, a fusion protein comprising the antibody of the disclosure and a cytotoxic or growth inhibitory polypeptide may be made, by recombinant techniques or peptide synthesis. The length of DNA may comprise respective regions encoding the two portions of the conjugate either adjacent one another or separated by a region encoding a linker peptide which does not destroy the desired properties of the conjugate.

The antibodies of the present disclosure may also be used in Dependent Enzyme Mediated Prodrug Therapy by conjugating the polypeptide to a prodrug-activating enzyme which converts a prodrug (e.g. a peptidyl chemotherapeutic agent, see WO81/01145) to an active anti-cancer drug (See, for example, WO 88/07378 and U.S. Pat. No. 4,975,278). The enzyme component of the immunoconjugate useful for ADEPT includes any enzyme capable of acting on a prodrug in such a way so as to convert it into its more active, cytotoxic form. Enzymes that are useful in the method of this disclosure include, but are not limited to, alkaline phosphatase useful for converting phosphate-containing prodrugs into free drugs; arylsulfatase useful for converting sulfate-containing prodrugs into free drugs; cytosine deaminase useful for converting non-toxic fluorocytosine into the anticancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as O-galactosidase and neuraminidase useful for converting glycosylated prodrugs into free drugs; P-lactamase useful for converting drugs derivatized with P-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. The enzymes can be covalently bound to the polypeptides of the disclosure by techniques well known in the art such as the use of the heterobifunctional crosslinking reagents discussed above.

According to an embodiment, in the conjugate of the disclosure, the growth inhibitory agent is a maytansinoid, in an embodiment DM1 or DM4.

In said conjugate, the antibody is conjugated to said at least one growth inhibitory agent by a linking group. In an embodiment said linking group is a cleavable or a non-cleavable linker, such as SPDB, sulfo-SPDB, or SMCC.

The conjugate may be selected from the group consisting of:
an antibody-SPDB-DM4 conjugate of formula (III)

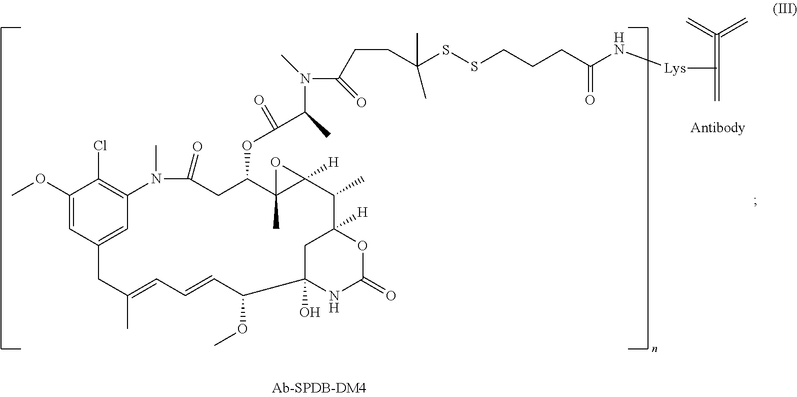

an antibody-sulfo-SPDB-DM4 conjugate of formula (IV)

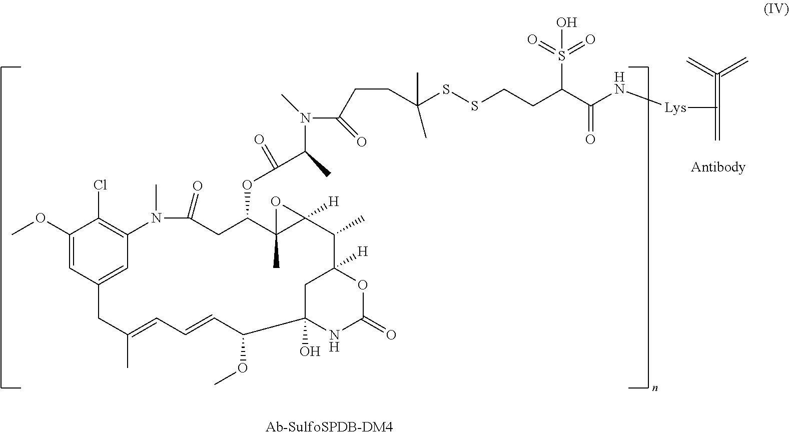

and
an antibody-SMCC-DM1 conjugate of formula (V)

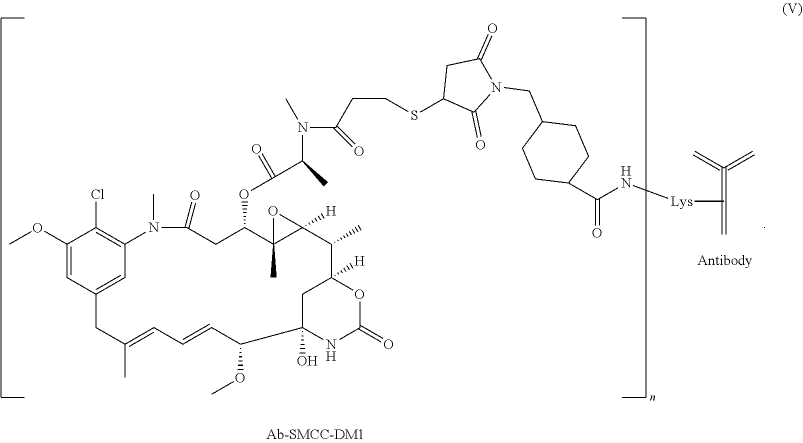

In an embodiment the conjugate is a conjugate of formula (III), (IV) or (V) as defined above, in which the antibody is an antibody described herein.

In formulas (III), (IV) and (V) above, “n” corresponds to the number of molecules of chemotherapeutic agent conjugated per molecule of antibody. It corresponds to the “drug-to-antibody ratio” (or “DAR”) defined herein and may range from 1 to 10.

In an embodiment, the conjugate is tusamitamab ravtansine (CAS Registry No. 2254086-60-5).

The conjugate tusamitamab ravtansine is also referred to as huMAb2-3-SPDB-DM4 in the Example section.

The conjugates of the disclosure may be prepared by in vitro methods. In general, the conjugate can be obtained by a process comprising the steps of:
(i) bringing into contact an optionally-buffered aqueous solution of an antibody according to the disclosure) with solutions of a linker and a cytotoxic compound; and
(ii) then optionally separating the conjugate which was formed in (i) from the unreacted antibody, linker and cytotoxic compounds.

The aqueous solution of cell-binding agent can be buffered with buffers such as, e.g. potassium phosphate, acetate, citrate or N-2-Hydroxyethylpiperazine-N′-2-ethanesulfonic acid (HEPES buffer). The buffer depends upon the nature of the cell-binding agent. The cytotoxic compound is in solution in an organic polar solvent, e.g., dimethyl sulfoxide (DMSO) or dimethylacetamide (DMA).

The reaction temperature is usually comprised between 2° and 40° C. The reaction time can vary from 1 to 24 hours. The reaction between the cell-binding agent and the cytotoxic agent can be monitored by size exclusion chromatography (SEC) with a refractometric and/or UV detector. If the conjugate yield is too low, the reaction time can be extended.

A number of different chromatography methods can be used by the person skilled in the art in order to perform the separation of step (ii): the conjugate can be purified e.g. by SEC, adsorption chromatography (such as ion exchange chromatography, IEC), hydrophobic interaction chromatography (HIC), affinity chromatography, mixed-support chromatography such as hydroxyapatite chromatography, or high performance liquid chromatography (HPLC). Purification by dialysis or diafiltration can also be used.

As used herein, the term “aggregates” means the associations which can be formed between two or more cell-binding agents, said agents being modified or not by conjugation. The aggregates can be formed under the influence of a great number of parameters, such as a high concentration of cell-binding agent in the solution, the pH of the solution, high shearing forces, the number of bonded dimers and their hydrophobic character, the temperature (see Wang & Gosh, 2008, J. Membrane Sci., 318:311-316, and references cited therein); note that the relative influence of some of these parameters is not clearly established. In the case of proteins and antibodies, the person skilled in the art will refer to Cromwell et al. (2006, AAPS Journal, 8 (3): E572-E579). The content in aggregates can be determined with techniques well known to the skilled person, such as SEC (see Walter et al., 1993, Anal. Biochem., 212 (2): 469-480).

After step (i) or (ii), the conjugate-containing solution can be submitted to an additional step (iii) of chromatography, ultrafiltration and/or diafiltration.

The conjugate is recovered at the end of these steps in an aqueous solution.

According to an embodiment, the conjugate according to the disclosure is characterised by a “drug-to-antibody ratio” (or “DAR”) ranging from 1 to 10, for instance from 2 to 5, or for example from 3 to 4. This is generally the case of conjugates including maytansinoid molecules.

This DAR number can vary with the nature of the antibody and of the drug (i.e. the growth-inhibitory agent) used along with the experimental conditions used for the conjugation (like the ratio growth-inhibitory agent/antibody, the reaction time, the nature of the solvent and of the cosolvent if any). Thus, the contact between the antibody and the growth-inhibitory agent leads to a mixture comprising several conjugates differing from one another by different drug-to-antibody ratios; optionally the naked antibody; optionally aggregates. The DAR that is determined is thus a mean value.

A method which can be used to determine the DAR consists in measuring spectrophotometrically the ratio of the absorbance at of a solution of substantially purified conjugate at λ_{D} and 280 nm. 280 nm is a wavelength generally used for measuring protein concentration, such as antibody concentration. The wavelength λ_{D} is selected so as to allow discriminating the drug from the antibody, i.e. as readily known to the skilled person, λ_{D} is a wavelength at which the drug has a high absorbance and λ_{D} is sufficiently remote from 280 nm to avoid substantial overlap in the absorbance peaks of the drug and antibody. λ_{D} may be selected as being 252 nm in the case of maytansinoid molecules. A method of DAR calculation may be derived from Antony S. Dimitrov (ed), LLC, 2009, Therapeutic Antibodies and Protocols, vol 525, 445, Springer Science:

The absorbances for the conjugate at λ_{D} (A_{λD}) and at 280 nm (A280) are measured either on the monomeric peak of the size exclusion chromatography (SEC) analysis (allowing to calculate the “DAR(SEC)” parameter) or using a classic spectrophotometer apparatus (allowing to calculate the “DAR(UV)” parameter). The absorbances can be expressed as follows:

$${A}_{λ D} = ( {c}_{D} \times {ε}_{D λ D} ) + ( {c}_{A} \times {ε}_{A λ D} )$$

$${A}_{280} = ( {c}_{D} \times {ε}_{D 280} ) + ( {c}_{A} \times {ε}_{A 280} )$$

wherein:
c_{D} and c_{A} are respectively the concentrations in the solution of the drug and of the antibody,
ε_{DλD} and ε_{D280} are respectively the molar extinction coefficients of the drug at λ_{D} and 280 nm, and
ε_{AλD} and ε_{A280} are respectively the molar extinction coefficients of the antibody at λ_{D} and 280 nm.

Resolution of these two equations with two unknowns leads to the following equations:

$${c}_{D} = [ ( {ε}_{A 280} \times {A}_{λ D} ) - ( {ε}_{A λ D} \times {A}_{280} ) ] / [ ( {ε}_{D λ D} \times {ε}_{A 280} ) - ( {ε}_{A λ D} \times {ε}_{D 280} ) ]$$

$${c}_{A} = [ {A}_{280} - ( {c}_{D} \times {ε}_{D 280} ) ] / {ε}_{A 280}$$

The average DAR is then calculated from the ratio of the drug concentration to that of the antibody: DAR=c_{D}/c_{A}.

### Anti-PD-1 Antibodies; Anti-PD-L1 Antibodies

Anti-PD-1 antibodies and anti-PD-L1 antibodies capable of interfering with interaction between PD-1, which is expressed on the surface of immune cells, and PD-L1, which is expressed on the surface of cancer cells, are useful as immune checkpoint inhibitors, thereby blocking a pathway that shields tumor cells from immune system components able and poised to fight cancer. When PD-1 and PD-L1 interact, they form a biochemical “shield” protecting tumor cells from being destroyed by the immune system. Thus, blockade of either PD-1 or PD-L1 leading to blockade of interaction between PD-1 and PD-L1 prevents or unmasks the biochemical “shield” protecting tumor cells from being destroyed by the immune system.

A number of anti-PD-1 antibodies have been approved for clinical use in the treatment of cancer. These include pembrolizumab (KEYTRUDA®), nivolumab (OPDIVO®), cemiplimab (LIBTAYO®), sintilimab (TYVYT®), dostarlimab (JEMPERLI®), and tislelizumab.

Similarly, a number of anti-PD-L1 antibodies have been approved for clinical use in the treatment of cancer. These include atezolizumab (TECENTRIQ®), avelumab (BAVENCIO®), and durvalumab (IMFINZI®).

### Platinum-Based Chemotherapies

Platinum-based anti-neoplastic agents are coordination complexes of platinum. These drugs are used to treat almost half of people receiving chemotherapy for cancer. In this form of chemotherapy, commonly used drugs include cisplatin, carboplatin, oxaliplatin, and nedaplatin. Their main mechanism of action is believed to be the induction of cancer cell apoptosis as a response to their covalent binding to DNA. In recent years, this picture has increased in complexity, based on studies indicating that cellular molecules other than DNA may potentially act as targets, and that part of the antitumor effects of platinum drugs occurs through modulation of the immune system. These immunogenic effects include modulation of STAT signaling; induction of an immunogenic type of cancer cell death through exposure of calreticulin and release of ATP and high-mobility group protein box-1 (HMGB-1); and enhancement of the effector immune response through modulation of programmed death receptor 1-ligand (PD-L1) and mannose-6-phosphate receptor expression. Both basic and clinical studies indicate that at least part of the antitumor efficacy of platinum-based chemotherapeutics may be due to immune potentiating mechanisms.

### Pemetrexed

Pemetrexed (N-[4-[2-(2-amino-4,7-dihydro-4-oxo-3H-pyrrolo[2,3-d]pyrimidin-5-yl) ethyl]benzoyl]-L-Glutamic acid) is an antimetabolite that inhibits at least three enzymes involved in the folate pathway: thymidylate synthase, dihydrofolate reductase, and glycinamide ribonucleotide formyltransferase. Pemetrexed has demonstrated clinical activity in non-small cell lung cancer as well as in a broad array of other solid tumors, including mesothelioma, breast, colorectal, bladder, cervical, gastric and pancreatic cancer. In September 2008, the FDA approved pemetrexed as a first-line treatment in combination with cisplatin against locally advanced and metastatic NSCLC in patients with non-squamous histology. Patients are recommended to take folic acid and vitamin B12 supplement even if levels are normal when they are on pemetrexed therapy.

### Pharmaceutical Compositions

The antibodies, immunoconjugates, and compounds of the disclosure may be combined with pharmaceutically acceptable excipients, and optionally sustained-release matrices, such as biodegradable polymers, to form therapeutic compositions.

Thus, another object of the disclosure relates to a pharmaceutical composition comprising an antibody, an immunoconjugate or a compound of the disclosure and a pharmaceutically acceptable carrier or excipient.

The disclosure also relates to an antibody, an immunoconjugate or a compound according to the disclosure, for use as a medicament.

The disclosure also relates to an antibody, an immunoconjugate or a compound according to the disclosure, for use as for treating cancer.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

As used herein, “pharmaceutically-acceptable carriers” includes any and all solvents, dispersion media, coatings, antibacterial and antifungal agents, and the like that are physiologically compatible. Examples of suitable carriers, diluents and/or excipients include one or more of water, amino acids, saline, phosphate buffered saline, buffer phosphate, acetate, citrate, succinate; amino acids and derivates such as histidine, arginine, glycine, proline, glycylglycine; inorganic salts NaCl, calcium chloride; sugars or polyalcohols such as dextrose, glycerol, ethanol, sucrose, trehalose, mannitol; surfactants such as Polysorbate 80, polysorbate 20, poloxamer 188; and the like, as well as combination thereof. In many cases, it will be preferable to include isotonic agents, such as sugars, polyalcohols, or sodium chloride in the composition, and formulation may also contain an antioxidant such as tryptamine and a stabilizing agent such as Tween 20.

The form of the pharmaceutical compositions, the route of administration, the dosage and the regimen naturally depend upon the condition to be treated, the severity of the illness, the age, weight, and gender of the patient, etc.

The pharmaceutical compositions of the disclosure can be formulated for a topical, oral, parenteral, intranasal, intravenous, intramuscular, subcutaneous or intraocular administration and the like.

In an embodiment, the pharmaceutical compositions contain vehicles which are pharmaceutically acceptable for a formulation capable of being injected. These may be isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical composition can be administrated through drug combination devices.

The doses used for the administration can be adapted as a function of various parameters, and for instance as a function of the mode of administration used, of the relevant pathology, or alternatively of the desired duration of treatment.

To prepare pharmaceutical compositions, an effective amount of the antibody or immunoconjugate of the disclosure may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and injectable with the appropriate device or system for delivery without degradation. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant. Dispersions can also be prepared in glycerol, liquid polyethylene glycols, and mixtures thereof and in oils. Under ordinary conditions of storage and use, these preparations contain a preservative to prevent the growth of microorganisms.

A polypeptide, antibody or immunoconjugate of the disclosure can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, glycine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with any of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more concentrated, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibody or immunoconjugate of the disclosure may be formulated within a therapeutic mixture to comprise about 0.01 to 100 milligrams, per dose or so.

In addition to the antibody or immunoconjugate formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of polypeptides into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles, or biodegradable polylactide or polylactide co glycolide nanoparticles that meet these requirements are contemplated for use in the present disclosure, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

### Methods of Treatment:

### Combination of Anti-CEACAM5 ADC and Anti-PD1/PDL1 Antibody:

An aspect of the disclosure is a method of treating a cancer, comprising administering to a subject in need thereof an effective amount of (i) an anti-CEACAM5 ADC and (ii) at least an anti-PD-1 antibody or an anti-PD-L1 antibody.

As used herein, an “effective amount” or “therapeutically effective amount” is a dose of the therapeutic that results in treatment of CEACAM5 expressing cancer (e.g., lung cancer, gastric cancer, gastroesophageal junction cancer, or esophageal cancer).

As used herein, “treating” refers to causing a detectable improvement in one or more symptoms associated with a CEACAM5 expressing cancer or causing a biological effect (e.g., a decrease in the level of a particular biomarker) that is correlated with the underlying pathologic mechanism(s) giving rise to the condition or symptom(s). For example, a dose of therapeutic which causes an improvement in any of the following symptoms or conditions associated with a CEACAM5 expressing cancer is deemed a “therapeutically effective amount.”

In another example, a treatment has not been effective when a dose of therapeutic does not result in a detectable improvement in one or more parameters or symptoms associated with a CEACAM5 expressing cancer (e.g., lung cancer, gastric cancer, gastroesophageal junction cancer, or esophageal cancer) or which does not cause a biological effect that is correlated with the underlying pathologic mechanism(s) giving rise to the condition or symptom(s) of cancer.

According to some of these embodiments, the anti-CEACAM5 ADC is administered intravenously.

In accordance with the methods of the present disclosure, a therapeutically effective amount of therapeutic that is administered to the subject will vary depending upon the age and the size (e.g., body weight or body surface area) of the subject as well as the route of administration and other factors well known to those of ordinary skill in the art.

An aspect of the disclosure is a method of treating a cancer, comprising administering to a subject in need thereof an effective amount of (i) an antibody-drug conjugate (ADC) comprising an anti-CEACAM5 antibody and (ii) an anti-PD-1 antibody or an anti-PD-L1 antibody, wherein the cancer expresses CEACAM5, thereby treating the cancer.

In an embodiment, the anti-CEACAM5 antibody comprises a HCDR1 having the amino acid sequence of SEQ ID NO: 1a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

HCDR1
(SEQ ID NO: 1)
GFVFSSYD
HCDR2
(SEQ ID NO: 2)
ISSGGGIT
HCDR3
(SEQ ID NO: 3)
AAHYFGSSGPFAY
LCDR1
(SEQ ID NO: 4)
ENIFSY
LCDR2
NTR
LCDR3
(SEQ ID NO: 5)
QHHYGTPFT

In certain embodiments, the anti-CEACAM5 antibody comprises a variable domain of a heavy chain (VH) consisting of SEQ ID NO: 6.

In certain embodiments, the anti-CEACAM5 antibody comprises a variable domain of a light chain (VL) consisting of SEQ ID NO: 7.

In certain embodiments, the anti-CEACAM5 antibody comprises a variable domain of a heavy chain (VH) consisting of SEQ ID NO: 6 and a variable domain of a light chain (VL) consisting of SEQ ID NO: 7.

(SEQ ID NO: 6)
EVQLQESGPGLVKPGGSLSLSCAASGFVFSSYDMSWVRQTP
ERGLEWVAYISSGGGITYAPSTVKGRFTVSRDNAKNTLYLQ
MNSLTSEDTAVYYCAAHYFGSSGPFAYVVGQGTLVTVSS
(SEQ ID NO: 7)
DIQMTQSPASLSASVGDRVTITCRASENIFSYLAWYQQKPG
KSPKLLVYNTRTLAEGVPSRFSGSGSGTDFSLTISSLQPED
FATYYCQHHYGTPFTFGSGTKLEI

In certain embodiments, the anti-CEACAM5 antibody comprises a heavy chain (HC) consisting of SEQ ID NO: 8.

In certain embodiments, the anti-CEACAM5 antibody comprises a light chain (LC) consisting of SEQ ID NO: 9.

In certain embodiments, the anti-CEACAM5 antibody comprises a heavy chain (HC) consisting of SEQ ID NO: 8 and a light chain (LC) consisting of SEQ ID NO: 9.

(SEQ ID NO: 8)
EVQLQESGPGLVKPGGSLSLSCAASGFVFSSYDMSWVRQTPERGL
EWVAYISSGGGITYAPSTVKGRFTVSRDNAKNTLYLQMNSLTSED
TAVYYCAAHYFGSSGPFAYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNYNHKPSNTKVDKKVEPKSCDK
THTCPPCPAPELLGGPSVFLFPPKPKDTLMISRTPEVTCVVVDVS
HEDPEVKFNWYVDGVEVHNAKTKPREEQYNSTYRWSVLTVLHQDW
LNGKEYKCKVSNKALPAPIEKTISKAKGQPREPQVYTLPPSRDEL
TKNQVSLTCLVKGFYPSDIAVEWESNGQPENNYKTTPPVLDSDGS
FFLYSKLTVDKSRWQQGNVFSCSVMHEALHNHYTQKSLSLSPG
(SEQ ID NO: 9)
DIQMTQSPASLSASVGDRVTITCRASENIFSYLAWYQQKPGKSPK
LLVYNTRTLAEGVPSRFSGSGSGTDFSLTISSLQPEDFATYYCQH
HYGTPFTFGSGTKLEIKRTVAAPSVFIFPPSDEQLKSGTASWCLL
NNFYPREAKVQWKVDNALQSGNSQESVTEQDSKDSTYSLSSTLTL
SKADYEKHKVYACEVTHQGLSSPVTKSFNRGEC

In certain embodiments, the anti-CEACAM5 antibody is tusamitamab.

In certain embodiments, the cytotoxic agent is selected from the group consisting of radioisotopes, protein toxins, small molecule toxins, and any combination thereof.

In certain embodiments, the small molecule toxin is selected from the group consisting of antimetabolites, DNA-alkylating agents, DNA-cross-linking agents, DNA-intercalating agents, anti-microtubule agents, topoisomerase inhibitors, and any combination thereof.

In certain embodiments, the anti-microtubule agent is selected from the group consisting of taxanes, vinca alkaloids, maytansinoids, colchicine, podophyllotoxin, griseofulvin, and any combination thereof.

In certain embodiments, the maytansinoid is selected from the group consisting of N2′-deacetyl-N2′-(3-mercapto-1-oxopropyl)-maytansine (DM1), N2′-deacetyl-N-2′(4-methyl-4-mercapto-1-oxopentyl)-maytansine (DM4), and any combination thereof.

In certain embodiments, the toxin is N2′-deacetyl-N2′-(3-mercapto-1-oxopropyl)-maytansine (DM1).

In certain embodiments, the toxin is N2′-deacetyl-N2′-(4-methyl-4-mercapto-1-oxopentyl)-maytansine (DM4).

In certain embodiments, the anti-CEACAM5-antibody is covalently attached via a cleavable or non-cleavable linker to the at least one cytotoxic agent.

In certain embodiments, the linker is selected from the group consisting of N-succinimidyl pyridyldithiobutyrate (SPDB), butanoic acid 4-[(5-nitro-2-pyridinyl) dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl(N-maleimidomethyl) cyclohexane-1-carboxylate (SMCC), and any combination thereof.

In certain embodiments, the toxin is covalently attached to the anti-CEACAM5 antibody directly.

In certain embodiments the toxin is covalently attached to the anti-CEACAM5 antibody by a linker consisting of N-succinimidyl pyridyldithiobutyrate (SPDB).

In certain embodiments the toxin is covalently attached to the anti-CEACAM5 antibody by a linker consisting of butanoic acid 4-[(5-nitro-2-pyridinyl) dithio]-2,5-dioxo-1-pyrrolidinyl ester (nitro-SPDB).

In certain embodiments the toxin is covalently attached to the anti-CEACAM5 antibody by a linker consisting of 4-(Pyridin-2-yldisulfanyl)-2-sulfo-butyric acid (sulfo-SPDB).

In certain embodiments the toxin is covalently attached to the anti-CEACAM5 antibody by a linker consisting of N-succinimidyl (2-pyridyldithio) propionate (SPDP).

In certain embodiments the toxin is covalently attached to the anti-CEACAM5 antibody by a linker consisting of succinimidyl (N-maleimidomethyl) cyclohexane-1-carboxylate (SMCC).

In certain embodiments, the ADC is characterized by a drug-to-antibody ratio (DAR) ranging from 1 to 10. In certain embodiments, the ADC has a DAR of 1. In certain embodiments, the ADC has a DAR of 2. In certain embodiments, the ADC has a DAR of 3. In certain embodiments, the ADC has a DAR of 4. In certain embodiments, the ADC has a DAR of 5. In certain embodiments, the ADC has a DAR of 6. In certain embodiments, the ADC has a DAR of 7. In certain embodiments, the ADC has a DAR of 8. In certain embodiments, the ADC has a DAR of 9. In certain embodiments, the ADC has a DAR of 10.

In certain embodiments, the ADC is characterized by a DAR of 2 to 5. In certain embodiments, the ADC is characterized by a DAR of 3 to 4.

In certain embodiments, the ADC is tusamitamab ravtansine.

In certain embodiments, the cancer expresses CEACAM5 with moderate or high intensity defined by immunohistochemistry (IHC).

In certain embodiments, immunohistochemical analysis can be performed on a contemporaneous sample or samples of tumor obtained from the subject. In certain embodiments, immunohistochemical analysis can be performed on a suitable historical sample or samples of tumor obtained from the subject.

In certain embodiments, the cancer expresses CEACAM5 with moderate intensity (immunohistochemistry intensity ≥2+ in ≥1% to <50% of tumor cells). In certain embodiments, the cancer expresses CEACAM5 with high intensity (immunohistochemistry intensity ≥2+ in ≥50% of tumor cells).

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g cholangiacarcinoma), prostate cancer, and skin cancer.

In certain embodiments, the cancer is selected from the group consisting of gastric cancer, gastroesophageal junction cancer, esophageal cancer and lung cancer. In certain embodiments, the cancer is gastric cancer.

In certain embodiments, the cancer is gastroesophageal junction cancer.

In certain embodiments, the cancer is esophageal cancer.

In certain embodiments, the cancer is lung cancer.

In certain embodiments, the lung cancer is nonsquamous non-small cell lung cancer (NSQ NSCLC).

In certain embodiments, the subject has advanced or metastatic NSQ NSCLC. In some embodiments, the subject has stage 3A NSQ NSCLC, e.g., where primary tumor has spread to the lymph nodes on the same side of the chest where it started. In some embodiments, the subject has stage 3B NSQ NSCLC, e.g., where primary tumor has spread to the lymph nodes on the same or opposite side of the chest where it started, above the collarbone, or in the space between the lungs. In some embodiments, the subject has stage 3C NSQ NSCLC, e.g., where large primary tumor has grown and spread to lymph nodes on the opposite side of the chest from where it started, above the collarbone, or in the space between the lungs, with two or more tumors on the same side of the chest. In some embodiments, the subject has stage 4 NSQ NSCLC, e.g., where there is metastasis to one or more sites outside of the chest. In some embodiments, the subject has widely metastatic NSQ NSCLC.

In certain embodiments, the subject has NSQ NSCLC with no epidermal growth factor receptor (EGFR) sensitizing mutation or v-raf murine sarcoma viral oncogene homolog B1 (BRAF) mutation or anaplastic lymphoma kinase/c-ros oncogene 1 (ALK/ROS) alterations. In certain embodiments, the subject has NSQ NSCLC with no EGFR sensitizing mutation. In certain embodiments, the subject has NSQ NSCLC with no BRAF mutation. In certain embodiments, the subject has NSQ NSCLC with no anaplastic lymphoma kinase/c-ros oncogene 1 (ALK/ROS) alterations. In certain embodiments, the subject has NSQ NSCLC without any combination of EGFR sensitizing mutation, BRAF mutation, and ALK/ROS alterations.

In certain embodiments, the subject has received no prior systemic chemotherapy for treatment of the cancer. In some embodiments, the subject has received no prior treatment with a platinum-based chemotherapy, e.g., cisplatin or carboplatin. In certain embodiments, the subject has received no prior treatment with pemetrexed.

In certain embodiments, the subject has received no prior immunotherapy for treatment of the cancer. Immunotherapy includes treatment with an immune checkpoint inhibitor, e.g., anti-PD-1 antibody or anti-PD-L1 antibody. In certain embodiments, the subject has received no prior treatment with an anti-PD-1 antibody or an anti-PD-L1 antibody. In certain embodiments, the subject has received no prior treatment with an anti-PD-L1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the anti-PD-1 antibody is nivolumab.

In certain embodiments, the anti-PD-1 antibody is cemiplimab.

In certain embodiments, the anti-PD-1 antibody is sintilimab.

In certain embodiments, the anti-PD-1 antibody is dostarlimab.

In certain embodiments, the anti-PD-1 antibody is tislelizumab.

In certain embodiments, the anti-PD-1 antibody is not pembrolizumab.

In certain embodiments, the anti-PD-1 antibody is not nivolumab.

In certain embodiments, the anti-PD-1 antibody is not cemiplimab.

In certain embodiments, the anti-PD-1 antibody is not sintilimab.

In certain embodiments, the anti-PD-1 antibody is not dostarlimab.

In certain embodiments, the anti-PD-1 antibody is not tislelizumab.

In certain embodiments, the anti-PD-L1 antibody is selected from the group consisting of atezolizumab, avelumab, and durvalumab.

In certain embodiments, the anti-PD-L1 antibody is atezolizumab.

In certain embodiments, the anti-PD-L1 antibody is avelumab.

In certain embodiments, the anti-PD-L1 antibody is durvalumab.

In certain embodiments, the anti-PD-L1 antibody is not atezolizumab.

In certain embodiments, the anti-PD-L1 antibody is not avelumab.

In certain embodiments, the anti-PD-L1 antibody is not durvalumab.

In certain embodiments, the ADC is tusamitamab ravtansine and the anti-PD-1 antibody is pembrolizumab.

### Anti-CEACAM5 ADC Dosing:

In certain embodiments, the dose of the ADC varies depending on the body surface area of the subject.

In certain embodiments, the dose of anti-CEACAM5 ADC administered to the subject is from about 1 mg/m² to about 500 mg/m².

In some embodiments, the dose of the ADC administered to the subject is from about 5 mg/m² to about 300 mg/m².

In various embodiments, the dose of the ADC administered to the subject is from about 5 mg/m² to about 250 mg/m².

In a further embodiment, the dose of the ADC administered to the subject is from about 60 mg/m² to about 190 mg/m².

In various embodiments, the dose is about 5, 10, 20, 30, 40, 60, 80, 100, 110, 120, 130, 140, 150, 160, 170, 180, 190, 200, or 210 mg/m² based on the body surface area of the subject.

In an embodiment, the dose of the ADC is 120 mg/m².

In an embodiment, the dose of the ADC is 150 mg/m².

In an embodiment, the dose of the ADC is 170 mg/m².

### Anti-PD1/PDL1 Dosing:

In general, anti-PD-1 or anti-PD-L1 antibodies can be administered in fixed doses (e.g., 200-400 mg) or on a per weight basis (e.g., 10 mg/kg), according to the type of cancer being treated. In certain embodiments, the anti-PD-1 or anti-PD-L1 administered intravenously. Intravenous administration can be an infusion or an injection. Typically, intravenous administration is an infusion.

In certain embodiments, the anti-PD-1 antibody or the anti-PD-L1 antibody is administered to the subject intravenously in a dose of about 200 mg to about 400 mg. In certain embodiments, the anti-PD-1 antibody or the anti-PD-L1 antibody is administered to the subject intravenously in a dose of 200 mg to 400 mg.

In certain embodiments, the anti-PD-1 antibody or the anti-PD-L1 antibody is administered to the subject intravenously in a dose of about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg.

In certain embodiments, the anti-PD-1 antibody or the anti-PD-L1 antibody is administered to the subject intravenously in a dose of 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, or 400 mg.

In certain embodiments, the anti-PD-1 antibody or the anti-PD-L1 antibody is administered to the subject intravenously in a dose selected from 200 mg, 350 mg, 360 mg, and 400 mg.

In certain embodiments, the anti-PD-1 antibody or the anti-PD-L1 antibody is administered to the subject intravenously in a dose selected from 200 mg.

In certain embodiments, the anti-PD-1 antibody or the anti-PD-L1 antibody is administered to the subject intravenously in a dose selected from 400 mg.

In certain exemplary embodiments, the PD1 antibody is pembrolizumab. In other exemplary embodiments, the PD1 antibody is sintilimab.

### Dosing Schedules for CEACAM5 ADC and Anti-PD1/PDL1 Combination Therapy:

In general, the combination of CEACAM5 ADC (e.g., tusamitamab ravtansine) and the anti-PD-1 antibody or the anti-PD-L1 antibody may be administered simultaneously or contemporaneously in any order. In certain embodiments, the anti-PD-1 antibody or the anti-PD-L1 antibody and the ADC are administered sequentially; that is, the anti-PD-1 antibody and the ADC are administered sequentially, or the anti-PD-L1 antibody and the ADC are administered sequentially. In certain embodiments, the anti-PD-1 antibody or the anti-PD-L1 antibody is administered before the ADC. In certain embodiments, the ADC is administered before the anti-PD-1 antibody or the anti-PD-L1 antibody.

In certain embodiments, there is a delay of at least about 30 minutes between the end of administration of the first agent and the start of administration of the second agent, provided the administration of both agents is completed within a single day or 24-hour period.

In certain embodiments, the anti-PD-1 antibody or the anti-PD-L1 antibody and the ADC are administered simultaneously; that is, the anti-PD-1 antibody and the ADC are administered simultaneously, or the anti-PD-L1 antibody and the ADC are administered simultaneously.

In certain embodiments, administration of both agents begins essentially simultaneously, e.g., within minutes of each other. In certain embodiments, administration of both agents ends essentially simultaneously, e.g., within minutes of each other. In certain embodiments, administration of both agents begins essentially simultaneously, e.g., within minutes of each other, and ends essentially simultaneously, e.g., within minutes of each other.

In certain embodiments, the anti-PD-1 antibody or the anti-PD-L1 antibody and the ADC are administered to the subject for at least four cycles; that is, the anti-PD-1 antibody and the ADC are administered to the subject for at least four cycles, or the anti-PD-L1 antibody and the ADC are administered to the subject for at least four cycles.

In certain embodiments, each cycle is about two to six weeks. In certain embodiments, each cycle is two to six weeks.

In certain embodiments, each cycle is about two weeks. In certain embodiments, each cycle is two weeks. In certain embodiments, each cycle is 12 to 17 days. In certain embodiments, a cycle is 12 days. In certain embodiments, a cycle is 13 days. In certain embodiments, a cycle is 14 days. In certain embodiments, a cycle is 15 days. In certain embodiments, a cycle is 16 days. In certain embodiments, a cycle is 17 days. In certain embodiments, at least one cycle is a day or two shorter or longer than at least one other cycle.

In certain embodiments, each cycle is about three weeks. In certain embodiments, each cycle is three weeks. In certain embodiments, each cycle is 18 to 24 days. In certain embodiments, a cycle is 18 days. In certain embodiments, a cycle is 19 days. In certain embodiments, a cycle is 20 days. In certain embodiments, a cycle is 21 days. In certain embodiments, a cycle is 22 days. In certain embodiments, a cycle is 23 days. In certain embodiments, a cycle is 24 days. In certain embodiments, at least one cycle is one to three days shorter or longer than at least one other cycle.

In certain embodiments, each cycle is about four weeks. In certain embodiments, each cycle is four weeks. In certain embodiments, each cycle is 25 to 32 days. In certain embodiments, a cycle is 25 days. In certain embodiments, a cycle is 26 days. In certain embodiments, a cycle is 27 days. In certain embodiments, a cycle is 28 days. In certain embodiments, a cycle is 29 days. In certain embodiments, a cycle is 30 days. In certain embodiments, a cycle is 31 days. In certain embodiments, a cycle is 32 days. In certain embodiments, at least one cycle is one to four days shorter or longer than at least one other cycle.

In certain embodiments, each cycle is about five weeks. In certain embodiments, each cycle is five weeks. In certain embodiments, each cycle is 33 to 40 days. In certain embodiments, a cycle is 33 days. In certain embodiments, a cycle is 34 days. In certain embodiments, a cycle is 35 days. In certain embodiments, a cycle is 36 days. In certain embodiments, a cycle is 37 days. In certain embodiments, a cycle is 38 days. In certain embodiments, a cycle is 39 days. In certain embodiments, a cycle is 40 days. In certain embodiments, at least one cycle is one to four days shorter or longer than at least one other cycle.

In certain embodiments, each cycle is about six weeks. In certain embodiments, each cycle is six weeks. In certain embodiments, each cycle is 36 to 48 days. In certain embodiments, a cycle is 36 days. In certain embodiments, a cycle is 37 days. In certain embodiments, a cycle is 38 days. In certain embodiments, a cycle is 39 days. In certain embodiments, a cycle is 40 days. In certain embodiments, a cycle is 41 days. In certain embodiments, a cycle is 42 days. In certain embodiments, a cycle is 43 days. In certain embodiments, a cycle is 44 days. In certain embodiments, a cycle is 45 days. In certain embodiments, a cycle is 46 days. In certain embodiments, a cycle is 47 days. In certain embodiments, a cycle is 48 days. In certain embodiments, at least one cycle is one to six days shorter or longer than at least one other cycle.

In certain embodiments, each tusamitamab ravtansine cycle is selected from the group consisting of: about two weeks, about three weeks, about four weeks, and about five weeks. In certain embodiments, each tusamitamab ravtansine cycle is about two weeks. In certain embodiments, each tusamitamab ravtansine cycle is about three weeks. In certain embodiments, each tusamitamab ravtansine cycle is about four weeks. In certain embodiments, each tusamitamab ravtansine cycle is about five weeks.

In certain embodiments, each tusamitamab ravtansine cycle is selected from the group consisting of: two weeks, three weeks, four weeks, and five weeks. In certain embodiments, each tusamitamab ravtansine cycle is two weeks. In certain embodiments, each tusamitamab ravtansine cycle is three weeks. In certain embodiments, each tusamitamab ravtansine cycle is four weeks. In certain embodiments, each tusamitamab ravtansine cycle is five weeks.

In certain embodiments, each anti-PD-1 antibody or anti-PD-L1 antibody cycle is selected from the group consisting of: about two weeks, about three weeks, and about six weeks. In certain embodiments, each anti-PD-1 antibody or anti-PD-L1 antibody cycle is about two weeks. In certain embodiments, each anti-PD-1 antibody or anti-PD-L1 antibody cycle is about three weeks. In certain embodiments, each anti-PD-1 antibody or anti-PD-L1 antibody cycle is about four weeks. In certain embodiments, each anti-PD-1 antibody or anti-PD-L1 antibody cycle is about five weeks. In certain embodiments, each anti-PD-1 antibody or anti-PD-L1 antibody cycle is about six weeks.

In certain embodiments, each anti-PD-1 antibody or anti-PD-L1 antibody cycle is two weeks. In certain embodiments, each anti-PD-1 antibody or anti-PD-L1 antibody cycle is three weeks. In certain embodiments, each anti-PD-1 antibody or anti-PD-L1 antibody cycle is four weeks. In certain embodiments, each anti-PD-1 antibody or anti-PD-L1 antibody cycle is five weeks. In certain embodiments, each anti-PD-1 antibody or anti-PD-L1 antibody cycle is six weeks.

In certain embodiments, each tusamitamab ravtansine cycle is two weeks and each anti-PD-1 antibody or anti-PD-L1 antibody cycle is two weeks. In certain embodiments, each tusamitamab ravtansine cycle is two weeks and each anti-PD-1 antibody or anti-PD-L1 antibody cycle is three weeks. In certain embodiments, each tusamitamab ravtansine cycle is two weeks and each anti-PD-1 antibody or anti-PD-L1 antibody cycle is six weeks.

In certain embodiments, each tusamitamab ravtansine cycle is three weeks and each anti-PD-1 antibody or anti-PD-L1 antibody cycle is two weeks. In certain embodiments, each tusamitamab ravtansine cycle is three weeks and each anti-PD-1 antibody or anti-PD-L1 antibody cycle is three weeks. In certain embodiments, each tusamitamab ravtansine cycle is three weeks and each anti-PD-1 antibody or anti-PD-L1 antibody cycle is six weeks.

In certain embodiments, each tusamitamab ravtansine cycle is four weeks and each anti-PD-1 antibody or anti-PD-L1 antibody cycle is two weeks. In certain embodiments, each tusamitamab ravtansine cycle is four weeks and each anti-PD-1 antibody or anti-PD-L1 antibody cycle is three weeks. In certain embodiments, each tusamitamab ravtansine cycle is four weeks and each anti-PD-1 antibody or anti-PD-L1 antibody cycle is six weeks.

In certain embodiments, each tusamitamab ravtansine cycle is four weeks and each anti-PD-1 antibody or anti-PD-L1 antibody cycle is two weeks. In certain embodiments, each tusamitamab ravtansine cycle is four weeks and each anti-PD-1 antibody or anti-PD-L1 antibody cycle is three weeks. In certain embodiments, each tusamitamab ravtansine cycle is four weeks and each anti-PD-1 antibody or anti-PD-L1 antibody cycle is six weeks.

In certain embodiments, each tusamitamab ravtansine cycle is six weeks and each anti-PD-1 antibody or anti-PD-L1 antibody cycle is two weeks. In certain embodiments, each tusamitamab ravtansine cycle is six weeks and each anti-PD-1 antibody or anti-PD-L1 antibody cycle is three weeks. In certain embodiments, each tusamitamab ravtansine cycle is six weeks and each anti-PD-1 antibody or anti-PD-L1 antibody cycle is six weeks.

In certain embodiments, the tusamitamab ravtansine is administered to the subject intravenously in a dose of about 60 mg/m² to about 190 mg/m². In certain embodiments, the tusamitamab ravtansine is administered to the subject intravenously in a dose of 60 mg/m² to 190 mg/m².

In certain embodiments, the tusamitamab ravtansine is administered to the subject intravenously in a dose of about 120 mg/m² to about 170 mg/m². In certain embodiments, the tusamitamab ravtansine is administered to the subject intravenously in a dose of 120 mg/m² to 170 mg/m².

In certain embodiments, the tusamitamab ravtansine is administered to the subject intravenously in a dose of about 120 mg/m².

In certain embodiments, the tusamitamab ravtansine is administered to the subject intravenously in a dose of about 130 mg/m².

In certain embodiments, the tusamitamab ravtansine is administered to the subject intravenously in a dose of about 140 mg/m².

In certain embodiments, the tusamitamab ravtansine is administered to the subject intravenously in a dose of about 150 mg/m².

In certain embodiments, the tusamitamab ravtansine is administered to the subject intravenously in a dose of about 160 mg/m².

In certain embodiments, the tusamitamab ravtansine is administered to the subject intravenously in a dose of about 170 mg/m².

In certain embodiments, the tusamitamab ravtansine is administered to the subject intravenously in a dose of 120 mg/m².

In certain embodiments, the tusamitamab ravtansine is administered to the subject intravenously in a dose of 130 mg/m².

In certain embodiments, the tusamitamab ravtansine is administered to the subject intravenously in a dose of 140 mg/m².

In certain embodiments, the tusamitamab ravtansine is administered to the subject intravenously in a dose of 150 mg/m².

In certain embodiments, the tusamitamab ravtansine is administered to the subject intravenously in a dose of 160 mg/m².

In certain embodiments, the tusamitamab ravtansine is administered to the subject intravenously in a dose of 170 mg/m².

In certain embodiments, the anti-PD-1 antibody or the anti-PD-L1 antibody and the tusamitamab ravtansine are administered about once every three weeks. In certain embodiments, the anti-PD-1 antibody or the anti-PD-L1 antibody and the tusamitamab ravtansine are administered once every three weeks.

In certain embodiments, the anti-PD-1 antibody or the anti-PD-L1 antibody and the tusamitamab ravtansine are administered about once every four weeks. In certain embodiments, the anti-PD-1 antibody or the anti-PD-L1 antibody and the tusamitamab ravtansine are administered once every four weeks.

In certain embodiments, the anti-PD-1 antibody or the anti-PD-L1 antibody and the tusamitamab ravtansine are administered about once every five weeks. In certain embodiments, the anti-PD-1 antibody or the anti-PD-L1 antibody and the tusamitamab ravtansine are administered once every five weeks.

In certain embodiments, the anti-PD-1 antibody or the anti-PD-L1 antibody and the tusamitamab ravtansine are administered about once every six weeks. In certain embodiments, the anti-PD-1 antibody or the anti-PD-L1 antibody and the tusamitamab ravtansine are administered once every six weeks.

In certain embodiments, the anti-PD-1 antibody or the anti-PD-L1 antibody is administered about once every three weeks and the tusamitamab ravtansine is administered about once every six weeks.

In certain embodiments, the anti-PD-1 antibody or the anti-PD-L1 antibody is administered about once every three weeks and the tusamitamab ravtansine is administered once every six weeks.

In certain embodiments, the anti-PD-1 antibody or the anti-PD-L1 antibody is administered once every three weeks and the tusamitamab ravtansine is administered once every six weeks.

In certain embodiments, the anti-PD-1 antibody or the anti-PD-L1 antibody is administered once every three weeks and the tusamitamab ravtansine is administered once every six weeks.

In certain embodiments, the anti-PD-1 antibody or the anti-PD-L1 antibody is administered about once every six weeks and the tusamitamab ravtansine is administered about once every three weeks.

In certain embodiments, the anti-PD-1 antibody or the anti-PD-L1 antibody is administered once every six weeks and the tusamitamab ravtansine is administered about once every three weeks.

In certain embodiments, the anti-PD-1 antibody or the anti-PD-L1 antibody is administered about once every six weeks and the tusamitamab ravtansine is administered once every three weeks.

In certain embodiments, the anti-PD-1 antibody or the anti-PD-L1 antibody is administered once every six weeks and the tusamitamab ravtansine is administered once every three weeks.

In certain embodiments, the anti-PD-1 antibody or the anti-PD-L1 antibody is administered to the subject intravenously in a dose of about 200 mg and the tusamitamab ravtansine is administered to the subject intravenously in a dose of about 120 mg/m² to about 170 mg/m².

In certain embodiments, the anti-PD-1 antibody or the anti-PD-L1 antibody is administered to the subject intravenously in a dose of 200 mg and the tusamitamab ravtansine is administered to the subject intravenously in a dose of 120 mg/m² to 170 mg/m².

In certain embodiments, the anti-PD-1 antibody or the anti-PD-L1 antibody is administered to the subject intravenously in a dose of about 400 mg and the tusamitamab ravtansine is administered to the subject intravenously in a dose of about 120 mg/m² to about 170 mg/m².

In certain embodiments, the anti-PD-1 antibody or the anti-PD-L1 antibody is administered to the subject intravenously in a dose of 400 mg and the tusamitamab ravtansine is administered to the subject intravenously in a dose of 120 mg/m² to 170 mg/m².

In certain embodiments, the anti-PD-1 antibody or the anti-PD-L1 antibody is administered to the subject intravenously in a dose of 200 mg and the tusamitamab ravtansine is administered to the subject intravenously in a dose of 120 mg/m².

In certain embodiments, the anti-PD-1 antibody or the anti-PD-L1 antibody is administered to the subject intravenously in a dose of 200 mg and the tusamitamab ravtansine is administered to the subject intravenously in a dose of 130 mg/m².

In certain embodiments, the anti-PD-1 antibody or the anti-PD-L1 antibody is administered to the subject intravenously in a dose of 200 mg and the tusamitamab ravtansine is administered to the subject intravenously in a dose of 140 mg/m².

In certain embodiments, the anti-PD-1 antibody or the anti-PD-L1 antibody is administered to the subject intravenously in a dose of 200 mg and the tusamitamab ravtansine is administered to the subject intravenously in a dose of 150 mg/m².

In certain embodiments, the anti-PD-1 antibody or the anti-PD-L1 antibody is administered to the subject intravenously in a dose of 200 mg and the tusamitamab ravtansine is administered to the subject intravenously in a dose of 160 mg/m².

In certain embodiments, the anti-PD-1 antibody or the anti-PD-L1 antibody is administered to the subject intravenously in a dose of 200 mg and the tusamitamab ravtansine is administered to the subject intravenously in a dose of 170 mg/m².

In certain embodiments, the anti-PD-1 antibody or the anti-PD-L1 antibody is administered to the subject intravenously in a dose of 400 mg and the tusamitamab ravtansine is administered to the subject intravenously in a dose of 120 mg/m².

In certain embodiments, the anti-PD-1 antibody or the anti-PD-L1 antibody is administered to the subject intravenously in a dose of 400 mg and the tusamitamab ravtansine is administered to the subject intravenously in a dose of 130 mg/m².

In certain embodiments, the anti-PD-1 antibody or the anti-PD-L1 antibody is administered to the subject intravenously in a dose of 400 mg and the tusamitamab ravtansine is administered to the subject intravenously in a dose of 140 mg/m².

In certain embodiments, the anti-PD-1 antibody or the anti-PD-L1 antibody is administered to the subject intravenously in a dose of 400 mg and the tusamitamab ravtansine is administered to the subject intravenously in a dose of 150 mg/m².

In certain embodiments, the anti-PD-1 antibody or the anti-PD-L1 antibody is administered to the subject intravenously in a dose of 400 mg and the tusamitamab ravtansine is administered to the subject intravenously in a dose of 160 mg/m².

In certain embodiments, the anti-PD-1 antibody or the anti-PD-L1 antibody is administered to the subject intravenously in a dose of 400 mg and the tusamitamab ravtansine is administered to the subject intravenously in a dose of 170 mg/m².

In certain embodiments, the anti-PD-1 antibody or the anti-PD-L1 antibody is administered to the subject followed by the tusamitamab ravtansine, i.e., first the anti-PD-1 antibody or the anti-PD-L1 antibody and then the tusamitamab ravtansine, wherein the anti-PD-1 antibody or the anti-PD-L1 antibody and the tusamitamab ravtansine are administered to the subject on Day 1 of a given cycle.

In certain embodiments, the anti-PD-1 antibody or the anti-PD-L1 antibody is administered to the subject followed by the tusamitamab ravtansine, i.e., first the anti-PD-1 antibody or the anti-PD-L1 antibody and then the tusamitamab ravtansine, wherein the anti-PD-1 antibody or the anti-PD-L1 antibody and the tusamitamab ravtansine are administered to the subject on Day 1 of each cycle.

In certain embodiments, the tusamitamab ravtansine is administered to the subject intravenously in a dose of about 120 mg/m². In certain embodiments, the tusamitamab ravtansine is administered to the subject intravenously in a dose of 120 mg/m².

In certain embodiments, the tusamitamab ravtansine is administered to the subject intravenously in a dose of about 150 mg/m². In certain embodiments, the tusamitamab ravtansine is administered to the subject intravenously in a dose of 150 mg/m².

In certain embodiments, the tusamitamab ravtansine is administered to the subject intravenously in a dose of about 170 mg/m². In certain embodiments, the tusamitamab ravtansine is administered to the subject intravenously in a dose of 170 mg/m².

In certain embodiments, the anti-PD-1 antibody or the anti-PD-L1 antibody is administered once every six weeks and the tusamitamab ravtansine is administered once every three weeks.

In certain embodiments, the ADC is tusamitamab ravtansine and the anti-PD-1 antibody is pembrolizumab.

An aspect of the disclosure is a method of treating a cancer, comprising administering to a subject in need thereof an effective amount of (i) an antibody-drug conjugate (ADC) that comprises an anti-CEACAM5 antibody and (ii) an anti-PD-1 antibody or an anti-PD-L1 antibody, thereby treating the cancer, wherein:

the cancer expresses CEACAM5 with high intensity (immunohistochemical intensity ≥2+ in ≥50% of tumor cells);

the anti-PD-1 antibody or the anti-PD-L1 antibody and the ADC are administered to the subject on a single day once a cycle, where each cycle is about three weeks.

An aspect of the disclosure is a method of treating a cancer, comprising administering to a subject in need thereof an effective amount of (i) an antibody-drug conjugate (ADC) that comprises an anti-CEACAM5 antibody and (ii) an anti-PD-1 antibody or anti-PD-L1 antibody, thereby treating the cancer, wherein:

the cancer expresses CEACAM5 with high intensity (immunohistochemical intensity ≥2+ in ≥50% of tumor cells);
the ADC is tusamitamab ravtansine;
the anti-PD-1 antibody or the anti-PD-L1 antibody and the tusamitamab ravtansine are administered to the subject on a single day once a cycle, where each cycle is about three weeks.

An aspect of the disclosure is a method of treating a cancer, comprising administering to a subject in need thereof an effective amount of (i) an antibody-drug conjugate (ADC) that comprises an anti-CEACAM5 antibody and (ii) an anti-PD-1 antibody or anti-PD-L1 antibody, thereby treating the cancer, wherein:
the cancer expresses CEACAM5 with high intensity (immunohistochemical intensity ≥2+ in ≥50% of tumor cells);
the ADC is tusamitamab ravtansine;
the anti-PD-1 antibody is pembrolizumab;
the pembrolizumab and the tusamitamab ravtansine are administered to the subject on a single day once a cycle, where each cycle is about three weeks.

An aspect of the disclosure is a method of treating a cancer, comprising administering to a subject in need thereof an effective amount of (i) an antibody-drug conjugate (ADC) that comprises an anti-CEACAM5 antibody and (ii) an anti-PD-1 antibody, thereby treating the cancer, wherein:
the cancer expresses CEACAM5 with high intensity (immunohistochemical intensity ≥2+ in ≥50% of tumor cells);
the ADC is tusamitamab ravtansine;
the anti-PD-1 antibody is pembrolizumab;
the pembrolizumab and the tusamitamab ravtansine are administered to the subject on a single day once a cycle, where each cycle is about three weeks;
wherein tusamitamab ravtansine is administered at a dose about 120 mg/m² to 170 mg/m² and pembrolizumab is administered at a dose about 200 mg/m².

### Triple Combination Therapy of Anti-CEACAM5 ADC, an Anti-PD1/PDL1 Antibody and a Platinum-Based Chemotherapy:

A further aspect of the disclosure is a method of treating a CEACAM5-expressing cancer in a subject in need thereof, comprising administering a triple combination therapy comprising a combination of an effective amount of (i) an antibody-drug conjugate (ADC) comprising an anti-CEACAM5 antibody, (ii) an anti-PD-1 antibody or an anti-PD-L1 antibody, and (iii) a platinum-based chemotherapy.

In certain embodiments, the platinum-based chemotherapy is selected from the group consisting of cisplatin and carboplatin.

In certain embodiments, the method comprises administering cisplatin to the subject.

In certain embodiments, the cisplatin is administered intravenously to the subject in a dose from 38 mg/m² to 75 mg/m².

In certain embodiments, the cisplatin is administered intravenously to the subject in a dose of about 75 mg/m².

In certain embodiments, the cisplatin is administered intravenously to the subject in a dose of about 75 mg/m² following administration of the ADC on Day 1 of each of cycles 1 to 4.

In certain embodiments, the method comprises administering carboplatin to the subject.

In certain embodiments, the carboplatin is administered intravenously to the subject in a dose of about (target AUC)×[(140−age)×(weight in kg)/serum creatinine (mg/dL)×72(×0.85 if female)+25] following administration of the ADC on Day 1 of each of cycles 1 to 4, wherein the target AUC is 5 mg*min/mL and the dose of carboplatin per administration is not to exceed 750 mg.

In certain embodiments, the carboplatin is administered intravenously to the subject in a dose of about (target AUC)×[(140−age)×(weight in kg)/serum creatinine (mg/dL)×72(×0.85 if female)+25], wherein the target AUC is from AUC 2.5 to AUC 5.

In certain embodiments, the target AUC is AUC 5.

In certain embodiments, the carboplatin is administered intravenously to the subject in a dose of (target AUC)×[(140−age)×(weight in kg)/serum creatinine (mg/dL)×72(×0.85 if female)+25] following administration of the pemetrexed on Day 1 of each of cycles 1 to 4, wherein the target AUC is 5 mg*min/mL and the dose of carboplatin per administration is not to exceed 750 mg.

In certain embodiments of the triple combination therapy, the anti-CEACAM5 antibody comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

In certain embodiments, the anti-CEACAM5 antibody comprises a variable domain of a heavy chain (VH) consisting of SEQ ID NO: 6 and a variable domain of a light chain (VL) consisting of SEQ ID NO: 7.

In certain embodiments, the anti-CEACAM5 antibody is tusamitamab.

In certain embodiments, the ADC comprises at least one cytotoxic agent.

In certain embodiments, the cytotoxic agent is selected from the group consisting of radioisotopes, protein toxins, small molecule toxins, and any combination thereof.

In certain embodiments, the small molecule toxin is selected from the group consisting of antimetabolites, DNA-alkylating agents, DNA-cross-linking agents, DNA-intercalating agents, anti-microtubule agents, topoisomerase inhibitors, and any combination thereof.

In certain embodiments, the anti-microtubule agent is selected from the group consisting of taxanes, vinca alkaloids, maytansinoids, colchicine, podophyllotoxin, griseofulvin, and any combination thereof.

In certain embodiments, the maytansinoid is selected from the group consisting of N2′-deacetyl-N2′-(3-mercapto-1-oxopropyl)-maytansine (DM1), N2′-deacetyl-N-2′(4-methyl-4-mercapto-1-oxopentyl)-maytansine (DM4), and any combination thereof.

In certain embodiments, the anti-CEACAM5-antibody is covalently attached via a cleavable or non-cleavable linker to the at least one cytotoxic agent.

In certain embodiments, the linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

In certain embodiments, the ADC is characterized by a drug-to-antibody ratio (DAR) ranging from 1 to 10.

In certain embodiments, the ADC is tusamitamab ravtansine.

In certain embodiments, wherein the cancer expresses CEACAM5 with moderate or high intensity defined by immunohistochemistry.

In certain embodiments, the cancer expresses CEACAM5 with moderate intensity (immunohistochemistry intensity ≥2+ in ≥1% to <50% of tumor cells).

In certain embodiments, the cancer expresses CEACAM5 with high intensity (immunohistochemistry intensity ≥2+ in ≥50% of tumor cells).

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g cholangiacarcinoma), prostate cancer, and skin cancer.

In certain embodiments, the cancer is gastric cancer, gastroesophageal junction cancer, or esophageal cancer.

In certain embodiments, the cancer is lung cancer.

In certain embodiments, the lung cancer is nonsquamous non-small cell lung cancer (NSQ NSCLC).

In some embodiments, the subject has stage 3A NSQ NSCLC, e.g., where primary tumor has spread to the lymph nodes on the same side of the chest where it started. In some embodiments, the subject has stage 3B NSQ NSCLC, e.g., where primary tumor has spread to the lymph nodes on the same or opposite side of the chest where it started, above the collarbone, or in the space between the lungs. In some embodiments, the subject has stage 3C NSQ NSCLC, e.g., where large primary tumor has grown and spread to lymph nodes on the opposite side of the chest from where it started, above the collarbone, or in the space between the lungs, with two or more tumors on the same side of the chest. In some embodiments, the subject has stage 4 NSQ NSCLC, e.g., where there is metastasis to one or more sites outside of the chest. In some embodiments, the subject has widely metastatic NSQ NSCLC.

In certain embodiments, the subject has NSQ NSCLC with no epidermal growth factor receptor (EGFR) sensitizing mutation or v-raf murine sarcoma viral oncogene homolog B1 (BRAF) mutation or anaplastic lymphoma kinase/c-ros oncogene 1 (ALK/ROS) alterations.

In certain embodiments, the subject has received no prior systemic chemotherapy for treatment of the cancer. In certain embodiments, the subject has received no prior systemic treatment with platinum, e.g., cisplatin or carboplatin.

In certain embodiments, the subject has received no prior immunotherapy for treatment of the cancer. Immunotherapy includes treatment with an immune checkpoint inhibitor, e.g., anti-PD-1 antibody or anti-PD-L1 antibody. In certain embodiments, the subject has received no prior treatment with an anti-PD-1 antibody or anti-PD-L1 antibody. In certain embodiments, the subject has received no prior treatment with an anti-PD-L1 antibody.

In certain embodiments, the anti-PD-1 antibody or anti-PD-L1 antibody is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the anti-PD-1 antibody or anti-PD-L1 antibody is an anti-PD-L1 antibody.

In certain embodiments, the anti-PD-L1 antibody is selected from the group consisting of atezolizumab, avelumab, and durvalumab.

In certain embodiments of the triple combination therapy, the anti-PD-1 antibody or the anti-PD-L1 antibody is administered before the ADC. In certain embodiments, the ADC is administered before the anti-PD-1 antibody or the anti-PD-L1 antibody. In certain embodiments, the anti-PD-1 antibody or the anti-PD-L1 antibody is administered before the ADC and the platinum-based chemotherapy. In certain embodiments, there is a delay of at least about 30 minutes between the end of administration of the first agent and the start of administration of the second agent, provided the administration of both agents is completed within a single day or 24-hour period.

In certain embodiments, the anti-PD-1 antibody or the anti-PD-L1 antibody and the ADC are administered simultaneously; that is, the anti-PD-1 antibody and the ADC are administered simultaneously, or the anti-PD-L1 antibody and the ADC are administered simultaneously. In certain embodiments, administration of both agents begins essentially simultaneously, e.g., within minutes of each other. In certain embodiments, administration of both agents ends essentially simultaneously, e.g., within minutes of each other. In certain embodiments, administration of both agents begins essentially simultaneously, e.g., within minutes of each other, and ends essentially simultaneously, e.g., within minutes of each other.

In certain embodiments, the NSQ NSCLC expresses CEACAM5 with high intensity (immunohistochemistry intensity ≥2+ in ≥50% of tumor cells).

Immunohistochemical analysis can be performed on a contemporaneous sample or samples of tumor obtained from the subject, or using a suitable historical sample or samples of tumor obtained from the subject.

In certain embodiments, the ADC is tusamitamab ravtansine.

In certain embodiments, the anti-PD-1 antibody is pembrolizumab.

In certain embodiments, the ADC is tusamitamab ravtansine and the anti-PD-1 antibody is pembrolizumab.

In certain embodiments of the triple combination therapy, each cycle is about two to six weeks. In certain embodiments, each cycle is two to six weeks.

In certain embodiments, each cycle is about two weeks. In certain embodiments, each cycle is two weeks.

In certain embodiments, each cycle is about three weeks. In certain embodiments, each cycle is three weeks.

In certain embodiments, each cycle is about six weeks. In certain embodiments, each cycle is six weeks.

In certain embodiments, each tusamitamab ravtansine cycle is selected from the group consisting of: two weeks, three weeks, and four weeks.

In certain embodiments, each anti-PD-1 antibody or anti-PD-L1 antibody cycle is selected from the group consisting of: two weeks, three weeks, and six weeks.

In certain embodiments, each tusamitamab ravtansine cycle is two weeks and each anti-PD-1 antibody or anti-PD-L1 antibody cycle is two weeks. In certain embodiments, each tusamitamab ravtansine cycle is two weeks and each anti-PD-1 antibody or anti-PD-L1 antibody cycle is three weeks. In certain embodiments, each tusamitamab ravtansine cycle is two weeks and each anti-PD-1 antibody or anti-PD-L1 antibody cycle is six weeks.

In certain embodiments, each tusamitamab ravtansine cycle is three weeks and each anti-PD-1 antibody or anti-PD-L1 antibody cycle is two weeks. In certain embodiments, each tusamitamab ravtansine cycle is three weeks and each anti-PD-1 antibody or anti-PD-L1 antibody cycle is three weeks. In certain embodiments, each tusamitamab ravtansine cycle is three weeks and each anti-PD-1 antibody or anti-PD-L1 antibody cycle is six weeks.

In certain embodiments, each tusamitamab ravtansine cycle is six weeks and each anti-PD-1 antibody or anti-PD-L1 antibody cycle is two weeks. In certain embodiments, each tusamitamab ravtansine cycle is six weeks and each anti-PD-1 antibody or anti-PD-L1 antibody cycle is three weeks. In certain embodiments, each tusamitamab ravtansine cycle is six weeks and each anti-PD-1 antibody or anti-PD-L1 antibody cycle is six weeks.

In certain embodiments, the anti-PD-1 antibody or the anti-PD-L1 antibody is administered to the subject intravenously in a dose of about 200 mg to about 400 mg. In certain embodiments, the anti-PD-1 antibody or the anti-PD-L1 antibody is administered to the subject intravenously in a dose of 200 mg to 400 mg.

In certain embodiments, the pembrolizumab is administered to the subject intravenously in a dose of about 200 mg to about 400 mg. In certain embodiments, the pembrolizumab is administered to the subject intravenously in a dose of 200 mg to 400 mg. The intravenous administration can be an infusion or an injection. Typically, intravenous administration is an infusion.

In certain embodiments, the pembrolizumab is administered to the subject intravenously in a dose of about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg.

In certain embodiments, the pembrolizumab is administered to the subject intravenously in a dose of 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, or 400 mg.

In certain embodiments, the anti-PD-1 antibody or the anti-PD-L1 antibody is administered to the subject intravenously in a dose selected from 200 mg, 350 mg, 360 mg, and 400 mg.

In certain embodiments, the tusamitamab ravtansine is administered to the subject intravenously in a dose of about 60 mg/m² to about 190 mg/m². In certain embodiments, the tusamitamab ravtansine is administered to the subject intravenously in a dose of 60 mg/m² to 190 mg/m².

In certain embodiments, the tusamitamab ravtansine is administered to the subject intravenously in a dose of about 120 mg/m² to about 170 mg/m². In certain embodiments, the tusamitamab ravtansine is administered to the subject intravenously in a dose of 120 mg/m² to 170 mg/m².

In certain embodiments, the tusamitamab ravtansine is administered to the subject intravenously in a dose of about 120 mg/m². In certain embodiments, the tusamitamab ravtansine is administered to the subject intravenously in a dose of 120 mg/m².

In certain embodiments, the tusamitamab ravtansine is administered to the subject intravenously in a dose of about 130 mg/m². In certain embodiments, the tusamitamab ravtansine is administered to the subject intravenously in a dose of 130 mg/m².

In certain embodiments, the tusamitamab ravtansine is administered to the subject intravenously in a dose of about 140 mg/m². In certain embodiments, the tusamitamab ravtansine is administered to the subject intravenously in a dose of 140 mg/m².

In certain embodiments, the tusamitamab ravtansine is administered to the subject intravenously in a dose of about 150 mg/m². In certain embodiments, the tusamitamab ravtansine is administered to the subject intravenously in a dose of 150 mg/m².

In certain embodiments, the tusamitamab ravtansine is administered to the subject intravenously in a dose of about 160 mg/m². In certain embodiments, the tusamitamab ravtansine is administered to the subject intravenously in a dose of 160 mg/m².

In certain embodiments, the tusamitamab ravtansine is administered to the subject intravenously in a dose of about 170 mg/m². In certain embodiments, the tusamitamab ravtansine is administered to the subject intravenously in a dose of 170 mg/m².

In certain embodiments, the anti-PD-1 antibody is administered to the subject intravenously in a dose of about 200 mg and the tusamitamab ravtansine is administered to the subject intravenously in a dose of about 120 mg/m² to about 170 mg/m². In certain embodiments, the anti-PD-1 antibody is administered to the subject intravenously in a dose of 200 mg and the tusamitamab ravtansine is administered to the subject intravenously in a dose of 120 mg/m² to 170 mg/m².

In certain embodiments, the pembrolizumab is administered to the subject intravenously in a dose of about 200 mg and the tusamitamab ravtansine is administered to the subject intravenously in a dose of about 120 mg/m² to about 170 mg/m². In certain embodiments, the pembrolizumab is administered to the subject intravenously in a dose of 200 mg and the tusamitamab ravtansine is administered to the subject intravenously in a dose of 120 mg/m² to 170 mg/m².

In certain embodiments, the anti-PD-1 antibody and the tusamitamab ravtansine are administered about once every three weeks. In certain embodiments, the anti-PD-1 antibody and the tusamitamab ravtansine are administered once every three weeks.

In certain embodiments, the pembrolizumab and the tusamitamab ravtansine are administered about once every three weeks. In certain embodiments, the pembrolizumab and the tusamitamab ravtansine are administered once every three weeks.

In certain embodiments, the pembrolizumab is administered about once every three weeks and the tusamitamab ravtansine is administered about once every six weeks. In certain embodiments, the pembrolizumab is administered about once every three weeks and the tusamitamab ravtansine is administered once every six weeks. In certain embodiments, the pembrolizumab is administered once every three weeks and the tusamitamab ravtansine is administered once every six weeks. In certain embodiments, the pembrolizumab is administered once every three weeks and the tusamitamab ravtansine is administered once every six weeks.

In certain embodiments, the pembrolizumab is administered about once every six weeks and the tusamitamab ravtansine is administered about once every three weeks. In certain embodiments, the pembrolizumab is administered once every six weeks and the tusamitamab ravtansine is administered about once every three weeks. In certain embodiments, the pembrolizumab is administered about once every six weeks and the tusamitamab ravtansine is administered once every three weeks. In certain embodiments, the pembrolizumab is administered once every six weeks and the tusamitamab ravtansine is administered once every three weeks.

In certain embodiments, the pembrolizumab and the tusamitamab ravtansine are administered about once every six weeks. In certain embodiments, the pembrolizumab and the tusamitamab ravtansine are administered once every six weeks.

In certain embodiments, the pembrolizumab is administered to the subject intravenously in a dose of about 400 mg and the tusamitamab ravtansine is administered to the subject intravenously in a dose of about 120 mg/m² to about 170 mg/m².

In certain embodiments, the pembrolizumab is administered to the subject intravenously in a dose of 400 mg and the tusamitamab ravtansine is administered to the subject intravenously in a dose of 120 mg/m² to 170 mg/m².

In certain embodiments, the pembrolizumab is administered to the subject intravenously in a dose of 400 mg and the tusamitamab ravtansine is administered to the subject intravenously in a dose of 120 mg/m². In certain embodiments, the pembrolizumab is administered to the subject intravenously in a dose of 400 mg and the tusamitamab ravtansine is administered to the subject intravenously in a dose of 130 mg/m². In certain embodiments, the pembrolizumab is administered to the subject intravenously in a dose of 400 mg and the tusamitamab ravtansine is administered to the subject intravenously in a dose of 140 mg/m². In certain embodiments, the pembrolizumab is administered to the subject intravenously in a dose of 400 mg and the tusamitamab ravtansine is administered to the subject intravenously in a dose of 150 mg/m². In certain embodiments, the pembrolizumab is administered to the subject intravenously in a dose of 400 mg and the tusamitamab ravtansine is administered to the subject intravenously in a dose of 160 mg/m². In certain embodiments, the pembrolizumab is administered to the subject intravenously in a dose of 400 mg and the tusamitamab ravtansine is administered to the subject intravenously in a dose of 170 mg/m².

In certain embodiments, the pembrolizumab is administered to the subject followed by the tusamitamab ravtansine, i.e., first the pembrolizumab and then the tusamitamab ravtansine, wherein the pembrolizumab and the tusamitamab ravtansine are administered to the subject on Day 1 of a given cycle.

In certain embodiments, the pembrolizumab is administered to the subject followed by the tusamitamab ravtansine, i.e., first the pembrolizumab and then the tusamitamab ravtansine, wherein the pembrolizumab and the tusamitamab ravtansine are administered to the subject on Day 1 of each cycle.

In certain embodiments, the tusamitamab ravtansine is administered to the subject intravenously in a dose of about 120 mg/m². In certain embodiments, the tusamitamab ravtansine is administered to the subject intravenously in a dose of 120 mg/m².

In certain embodiments, the tusamitamab ravtansine is administered to the subject intravenously in a dose of about 150 mg/m². In certain embodiments, the tusamitamab ravtansine is administered to the subject intravenously in a dose of 150 mg/m².

In certain embodiments, the tusamitamab ravtansine is administered to the subject intravenously in a dose of about 170 mg/m². In certain embodiments, the tusamitamab ravtansine is administered to the subject intravenously in a dose of 170 mg/m².

In certain embodiments, the pembrolizumab is administered to the subject intravenously in a dose of about 200 mg and the tusamitamab ravtansine is administered to the subject intravenously in a dose of about 120 mg/m² to about 170 mg/m².

In certain embodiments, the pembrolizumab is administered to the subject intravenously in a dose of 200 mg and the tusamitamab ravtansine is administered to the subject intravenously in a dose of 120 mg/m² to 170 mg/m².

Quadruple Combination Therapy of (i) Anti-CEACAM5 ADC, (ii) an Anti-PD1/PDL1 Antibody, (iii) a Platinum-Based Chemotherapy, and (iv) Pemetrexed:

In yet another aspect, the disclosure provides a method of treating a CEACAM5-expressing cancer in a subject in need thereof, comprising administering a quadruple combination therapy comprising a combination of an effective amount of (i) an antibody-drug conjugate (ADC) comprising an anti-CEACAM5 antibody, (ii) an anti-PD-1 antibody or an anti-PD-L1 antibody, (iii) a platinum-based chemotherapy and (iv) pemetrexed.

In certain embodiments, the pemetrexed is administered intravenously at a dose from 250 mg/m² to 500 mg/m².

In certain embodiments, the pemetrexed is administered intravenously at a dose of about 250 mg/m². In certain embodiments, the pemetrexed is administered intravenously at a dose of 250 mg/m².

In certain embodiments, the pemetrexed is administered intravenously at a dose of about 300 mg/m². In certain embodiments, the pemetrexed is administered intravenously at a dose of 300 mg/m².

In certain embodiments, the pemetrexed is administered intravenously at a dose of about 350 mg/m². In certain embodiments, the pemetrexed is administered intravenously at a dose of 350 mg/m².

In certain embodiments, the pemetrexed is administered intravenously at a dose of about 400 mg/m². In certain embodiments, the pemetrexed is administered intravenously at a dose of 400 mg/m².

In certain embodiments, the pemetrexed is administered intravenously at a dose of about 450 mg/m². In certain embodiments, the pemetrexed is administered intravenously at a dose of 450 mg/m².

In certain embodiments, the pemetrexed is administered intravenously at a dose of about 500 mg/m². In certain embodiments, the pemetrexed is administered intravenously at a dose of 500 mg/m².

In certain embodiments, the pemetrexed is administered intravenously after a vitamin supplementation.

In certain embodiments, the pemetrexed is administered intravenously to the subject in a dose of about 500 mg/m² following administration of the ADC on Day 1 of a given cycle. In certain embodiments, the pemetrexed is administered intravenously to the subject in a dose of 500 mg/m² following administration of the ADC on Day 1 of a given cycle.

In certain embodiments, the pemetrexed is administered intravenously to the subject in a dose of about 500 mg/m² following administration of the ADC on Day 1 of each cycle. In certain embodiments, the pemetrexed is administered intravenously to the subject in a dose of 500 mg/m² following administration of the ADC on Day 1 of each cycle.

In certain embodiments of the quadruple combination therapy, the anti-CEACAM5 antibody of the ADC comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

In certain embodiments, the anti-CEACAM5 antibody comprises a variable domain of a heavy chain (VH) consisting of SEQ ID NO: 6 and a variable domain of a light chain (VL) consisting of SEQ ID NO: 7.

In certain embodiments, the anti-CEACAM5 antibody is tusamitamab.

In certain embodiments, the ADC comprises at least one cytotoxic agent.

In certain embodiments, the cytotoxic agent is selected from the group consisting of radioisotopes, protein toxins, small molecule toxins, and any combination thereof.

In certain embodiments, the small molecule toxin is selected from the group consisting of antimetabolites, DNA-alkylating agents, DNA-cross-linking agents, DNA-intercalating agents, anti-microtubule agents, topoisomerase inhibitors, and any combination thereof.

In certain embodiments, the anti-microtubule agent is selected from the group consisting of taxanes, vinca alkaloids, maytansinoids, colchicine, podophyllotoxin, griseofulvin, and any combination thereof.

In certain embodiments, the maytansinoid is selected from the group consisting of N2′-deacetyl-N2′-(3-mercapto-1-oxopropyl)-maytansine (DM1), N2′-deacetyl-N-2′(4-methyl-4-mercapto-1-oxopentyl)-maytansine (DM4), and any combination thereof.

In certain embodiments, the toxin is N2′-deacetyl-N2′-(4-methyl-4-mercapto-1-oxopentyl)-maytansine (DM4).

In certain embodiments, the anti-CEACAM5-antibody is covalently attached via a cleavable or non-cleavable linker to the at least one cytotoxic agent.

In certain embodiments, the linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

In certain embodiments, the toxin is covalently attached to the anti-CEACAM5 antibody by an N-succinimidyl pyridyldithiobutyrate (SPDB) linker.

In certain embodiments, the toxin is DM4 and the toxin is covalently attached to the anti-CEACAM5 antibody by an SPDB linker

In certain embodiments, the ADC is characterized by a drug-to-antibody ratio (DAR) ranging from 1 to 10.

In certain embodiments, the ADC is tusamitamab ravtansine.

In certain embodiments, the cancer expresses CEACAM5 with moderate or high intensity defined by immunohistochemistry.

In certain embodiments, the cancer expresses CEACAM5 with moderate intensity (immunohistochemistry intensity ≥2+ in ≥1% to <50% of tumor cells).

In certain embodiments, the cancer expresses CEACAM5 with high intensity (immunohistochemistry intensity ≥2+ in ≥50% of tumor cells).

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g cholangiacarcinoma), prostate cancer, and skin cancer.

In certain embodiments, the cancer is gastric cancer, gastroesophageal junction cancer, or esophageal cancer.

In certain embodiments, the cancer is lung cancer.

In certain embodiments, the lung cancer is nonsquamous non-small cell lung cancer (NSQ NSCLC).

In certain embodiments, the subject has advanced or metastatic NSQ NSCLC.

In certain embodiments, the subject has NSQ NSCLC with no epidermal growth factor receptor (EGFR) sensitizing mutation or v-raf murine sarcoma viral oncogene homolog B1 (BRAF) mutation or anaplastic lymphoma kinase/c-ros oncogene 1 (ALK/ROS) alterations.

In certain embodiments, the subject has received no prior systemic chemotherapy for treatment of the cancer. In certain embodiments, the subject has received no prior systemic chemotherapy for treatment of the NSQ NSCLC.

In certain embodiments, the subject has received no prior systemic treatment with platinum, e.g., cisplatin or carboplatin.

In certain embodiments, the subject has received no prior systemic treatment with pemetrexed.

In certain embodiments, the subject has received no prior immunotherapy for treatment of the cancer. Immunotherapy includes treatment with an immune checkpoint inhibitor, e.g., an anti-PD-1 antibody or an anti-PD-L1 antibody. In certain embodiments, the subject has received no prior treatment with an anti-PD-1 antibody. In certain embodiments, the subject has received no prior treatment with an anti-PD-L1 antibody.

In certain embodiments, the anti-PD-1 antibody or anti-PD-L1 antibody is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the anti-PD-1 antibody or anti-PD-L1 antibody is an anti-PD-L1 antibody.

In certain embodiments, the anti-PD-L1 antibody is selected from the group consisting of atezolizumab, avelumab, and durvalumab.

In certain embodiments, the platinum-based chemotherapy is selected from the group consisting of cisplatin and carboplatin.

In certain embodiments, the anti-PD-1 antibody or the anti-PD-L1 antibody and the ADC are administered sequentially; that is, the anti-PD-1 antibody and the ADC are administered sequentially, or the anti-PD-L1 antibody and the ADC are administered sequentially. In certain embodiments, the anti-PD-1 antibody or the anti-PD-L1 antibody is administered before the ADC. In certain embodiments, the ADC is administered before the anti-PD-1 antibody or the anti-PD-L1 antibody. In certain embodiments, the anti-PD-1 antibody or the anti-PD-L1 antibody is administered before the ADC, the platinum-based chemotherapy, and the pemetrexed.

In certain embodiments, there is a delay of at least about 30 minutes between the end of administration of the first agent and the start of administration of the second agent, provided the administration of both agents is completed within a single day or 24-hour period.

In certain embodiments, the anti-PD-1 antibody or the anti-PD-L1 antibody and the ADC are administered simultaneously; that is, the anti-PD-1 antibody and the ADC are administered simultaneously, or the anti-PD-L1 antibody and the ADC are administered simultaneously. In certain embodiments, administration of both agents begins essentially simultaneously, e.g., within minutes of each other. In certain embodiments, administration of both agents ends essentially simultaneously, e.g., within minutes of each other. In certain embodiments, administration of both agents begins essentially simultaneously, e.g., within minutes of each other, and ends essentially simultaneously, e.g., within minutes of each other.

In certain embodiments, the NSQ NSCLC expresses CEACAM5 with at least moderate intensity (immunohistochemistry intensity ≥2+ in ≥1% to <50% of tumor cells).

In certain embodiments, the NSQ NSCLC expresses CEACAM5 with high intensity (immunohistochemistry intensity ≥2+ in ≥50% of tumor cells).

Immunohistochemical analysis can be performed on a contemporaneous sample or samples of tumor obtained from the subject, or using a suitable historical sample or samples of tumor obtained from the subject.

In certain embodiments, the subject has advanced or metastatic NSQ NSCLC. In some embodiments, the subject has stage 3A NSQ NSCLC, e.g., where primary tumor has spread to the lymph nodes on the same side of the chest where it started. In some embodiments, the subject has stage 3B NSQ NSCLC, e.g., where primary tumor has spread to the lymph nodes on the same or opposite side of the chest where it started, above the collarbone, or in the space between the lungs. In some embodiments, the subject has stage 3C NSQ NSCLC, e.g., where large primary tumor has grown and spread to lymph nodes on the opposite side of the chest from where it started, above the collarbone, or in the space between the lungs, with two or more tumors on the same side of the chest. In some embodiments, the subject has stage 4 NSQ NSCLC, e.g., where there is metastasis to one or more sites outside of the chest. In some embodiments, the subject has widely metastatic NSQ NSCLC.

In certain embodiments, the subject has NSQ NSCLC with no epidermal growth factor receptor (EGFR) sensitizing mutation or v-raf murine sarcoma viral oncogene homolog B1 (BRAF) mutation or anaplastic lymphoma kinase/c-ros oncogene 1 (ALK/ROS) alterations.

In certain embodiments, the subject has NSQ NSCLC with no EGFR sensitizing mutation. In certain embodiments, the subject has NSQ NSCLC with no BRAF mutation. In certain embodiments, the subject has NSQ NSCLC with no anaplastic lymphoma kinase/c-ros oncogene 1 (ALK/ROS) alterations. In certain embodiments, the subject has NSQ NSCLC without any combination of EGFR sensitizing mutation, BRAF mutation, and ALK/ROS alterations.

In certain embodiments, the ADC is tusamitamab ravtansine.

In certain embodiments, the anti-PD-1 antibody is pembrolizumab.

In certain embodiments, the ADC is tusamitamab ravtansine and the anti-PD-1 antibody is pembrolizumab.

In certain embodiments, the anti-PD-1 antibody or the anti-PD-L1 antibody, the ADC, and the platinum-based chemotherapy are administered to the subject for at least four cycles.

In certain embodiments, each cycle is about two to six weeks. In certain embodiments, each cycle is two to six weeks.

In certain embodiments, each cycle is about two weeks. In certain embodiments, each cycle is two weeks.

In certain embodiments, each cycle is about three weeks. In certain embodiments, each cycle is three weeks.

In certain embodiments, each cycle is about six weeks. In certain embodiments, each cycle is six weeks.

In certain embodiments, each tusamitamab ravtansine cycle is selected from the group consisting of: two weeks, three weeks, and four weeks.

In certain embodiments, each anti-PD-1 or anti-PD-L1 cycle is selected from the group consisting of: two weeks, three weeks, and six weeks.

In certain embodiments, the ADC is tusamitamab ravtansine.

In certain embodiments, the anti-PD-1 antibody or the anti-PD-L1 antibody is administered to the subject intravenously in a dose of about 200 mg to about 400 mg. In certain embodiments, the anti-PD-1 antibody or the anti-PD-L1 antibody is administered to the subject intravenously in a dose of 200 mg to 400 mg. The intravenous administration can be an infusion or an injection. Typically, intravenous administration is an infusion.

In certain embodiments, the pembrolizumab is administered to the subject intravenously in a dose of about 200 mg to about 400 mg. In certain embodiments, the pembrolizumab is administered to the subject intravenously in a dose of 200 mg to 400 mg. The intravenous administration can be an infusion or an injection. Typically, intravenous administration is an infusion.

In certain embodiments, the pembrolizumab is administered to the subject intravenously in a dose of about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg.

In certain embodiments, the pembrolizumab is administered to the subject intravenously in a dose of 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, or 400 mg.

In certain embodiments, the anti-PD-1 antibody or the anti-PD-L1 antibody is administered to the subject intravenously in a dose selected from 200 mg, 350 mg, 360 mg, and 400 mg.

In certain embodiments, the tusamitamab ravtansine is administered to the subject intravenously in a dose of about 60 mg/m² to about 190 mg/m². In certain embodiments, the tusamitamab ravtansine is administered to the subject intravenously in a dose of 60 mg/m² to 190 mg/m².

In certain embodiments, the tusamitamab ravtansine is administered to the subject intravenously in a dose of about 120 mg/m² to about 170 mg/m². In certain embodiments, the tusamitamab ravtansine is administered to the subject intravenously in a dose of 120 mg/m² to 170 mg/m².

In certain embodiments, the tusamitamab ravtansine is administered to the subject intravenously in a dose of about 120 mg/m². In certain embodiments, the tusamitamab ravtansine is administered to the subject intravenously in a dose of 120 mg/m².

In certain embodiments, the tusamitamab ravtansine is administered to the subject intravenously in a dose of about 130 mg/m². In certain embodiments, the tusamitamab ravtansine is administered to the subject intravenously in a dose of 130 mg/m².

In certain embodiments, the tusamitamab ravtansine is administered to the subject intravenously in a dose of about 140 mg/m². In certain embodiments, the tusamitamab ravtansine is administered to the subject intravenously in a dose of 140 mg/m².

In certain embodiments, the tusamitamab ravtansine is administered to the subject intravenously in a dose of about 150 mg/m². In certain embodiments, the tusamitamab ravtansine is administered to the subject intravenously in a dose of 150 mg/m².

In certain embodiments, the tusamitamab ravtansine is administered to the subject intravenously in a dose of about 160 mg/m². In certain embodiments, the tusamitamab ravtansine is administered to the subject intravenously in a dose of 160 mg/m².

In certain embodiments, the tusamitamab ravtansine is administered to the subject intravenously in a dose of about 170 mg/m². In certain embodiments, the tusamitamab ravtansine is administered to the subject intravenously in a dose of 170 mg/m².

In certain embodiments, the anti-PD-1 antibody or the anti-PD-L1 antibody is administered to the subject intravenously in a dose of about 200 mg and the tusamitamab ravtansine is administered to the subject intravenously in a dose of about 120 mg/m² to about 170 mg/m². In certain embodiments, the anti-PD-1 antibody or the anti-PD-L1 antibody is administered to the subject intravenously in a dose of 200 mg and the tusamitamab ravtansine is administered to the subject intravenously in a dose of 120 mg/m² to 170 mg/m².

In certain embodiments, the pembrolizumab is administered to the subject intravenously in a dose of about 200 mg and the tusamitamab ravtansine is administered to the subject intravenously in a dose of about 120 mg/m² to about 170 mg/m².

In certain embodiments, the pembrolizumab is administered to the subject intravenously in a dose of 200 mg and the tusamitamab ravtansine is administered to the subject intravenously in a dose of 120 mg/m² to 170 mg/m².

In certain embodiments, the anti-PD-1 antibody or the anti-PD-L1 antibody and the tusamitamab ravtansine are administered about once every three weeks. In certain embodiments, the anti-PD-1 antibody or the anti-PD-L1 antibody and the tusamitamab ravtansine are administered once every three weeks.

In certain embodiments, the pembrolizumab and the tusamitamab ravtansine are administered about once every three weeks. In certain embodiments, the pembrolizumab and the tusamitamab ravtansine are administered once every three weeks.

In certain embodiments, the pembrolizumab is administered about once every three weeks and the tusamitamab ravtansine is administered about once every six weeks. In certain embodiments, the pembrolizumab is administered about once every three weeks and the tusamitamab ravtansine is administered once every six weeks. In certain embodiments, the pembrolizumab is administered once every three weeks and the tusamitamab ravtansine is administered once every six weeks. In certain embodiments, the pembrolizumab is administered once every three weeks and the tusamitamab ravtansine is administered once every six weeks.

In certain embodiments, the pembrolizumab is administered about once every six weeks and the tusamitamab ravtansine is administered about once every three weeks. In certain embodiments, the pembrolizumab is administered once every six weeks and the tusamitamab ravtansine is administered about once every three weeks. In certain embodiments, the pembrolizumab is administered about once every six weeks and the tusamitamab ravtansine is administered once every three weeks. In certain embodiments, the pembrolizumab is administered once every six weeks and the tusamitamab ravtansine is administered once every three weeks.

In certain embodiments, the anti-PD-1 antibody or the anti-PD-L1 antibody is administered to the subject intravenously in a dose of about 400 mg and the tusamitamab ravtansine is administered to the subject intravenously in a dose of about 120 mg/m² to about 170 mg/m².

In certain embodiments, the anti-PD-1 antibody or the anti-PD-L1 antibody is administered to the subject intravenously in a dose of 400 mg and the tusamitamab ravtansine is administered to the subject intravenously in a dose of 120 mg/m² to 170 mg/m².

In certain embodiments, the pembrolizumab is administered to the subject intravenously in a dose of about 400 mg and the tusamitamab ravtansine is administered to the subject intravenously in a dose of about 120 mg/m² to about 170 mg/m².

In certain embodiments, the pembrolizumab is administered to the subject intravenously in a dose of 400 mg and the tusamitamab ravtansine is administered to the subject intravenously in a dose of 120 mg/m² to 170 mg/m².

In certain embodiments, the pembrolizumab is administered to the subject intravenously in a dose of 400 mg and the tusamitamab ravtansine is administered to the subject intravenously in a dose of 120 mg/m². In certain embodiments, the pembrolizumab is administered to the subject intravenously in a dose of 400 mg and the tusamitamab ravtansine is administered to the subject intravenously in a dose of 130 mg/m². In certain embodiments, the pembrolizumab is administered to the subject intravenously in a dose of 400 mg and the tusamitamab ravtansine is administered to the subject intravenously in a dose of 140 mg/m². In certain embodiments, the pembrolizumab is administered to the subject intravenously in a dose of 400 mg and the tusamitamab ravtansine is administered to the subject intravenously in a dose of 150 mg/m². In certain embodiments, the pembrolizumab is administered to the subject intravenously in a dose of 400 mg and the tusamitamab ravtansine is administered to the subject intravenously in a dose of 160 mg/m². In certain embodiments, the pembrolizumab is administered to the subject intravenously in a dose of 400 mg and the tusamitamab ravtansine is administered to the subject intravenously in a dose of 170 mg/m².

In certain embodiments, the anti-PD-1 antibody or the anti-PD-L1 antibody and the tusamitamab ravtansine are administered about once every six weeks. In certain embodiments, the anti-PD-1 antibody or the anti-PD-L1 antibody and the tusamitamab ravtansine are administered once every six weeks.

In certain embodiments, the pembrolizumab and the tusamitamab ravtansine are administered about once every six weeks. In certain embodiments, the pembrolizumab and the tusamitamab ravtansine are administered once every six weeks.

In certain embodiments, the anti-PD-1 antibody or the anti-PD-L1 antibody is administered once every six weeks and the tusamitamab ravtansine is administered once every three weeks.

In certain embodiments, the pembrolizumab is administered to the subject followed by the tusamitamab ravtansine, i.e., first the pembrolizumab and then the tusamitamab ravtansine, wherein the pembrolizumab and the tusamitamab ravtansine are administered to the subject on Day 1 of a given cycle.

In certain embodiments, the pembrolizumab is administered to the subject followed by the tusamitamab ravtansine, i.e., first the pembrolizumab and then the tusamitamab ravtansine, wherein the pembrolizumab and the tusamitamab ravtansine are administered to the subject on Day 1 of each cycle.

In certain embodiments, the tusamitamab ravtansine is administered to the subject intravenously in a dose of about 120 mg/m². In certain embodiments, the tusamitamab ravtansine is administered to the subject intravenously in a dose of 120 mg/m².

In certain embodiments, the tusamitamab ravtansine is administered to the subject intravenously in a dose of about 150 mg/m². In certain embodiments, the tusamitamab ravtansine is administered to the subject intravenously in a dose of 150 mg/m².

In certain embodiments, the tusamitamab ravtansine is administered to the subject intravenously in a dose of about 170 mg/m². In certain embodiments, the tusamitamab ravtansine is administered to the subject intravenously in a dose of 170 mg/m².

In certain embodiments, the method comprises administering cisplatin to the subject.

In certain embodiments, the cisplatin is administered intravenously to the subject in a dose from 38 mg/m² to 75 mg/m².

In certain embodiments, the cisplatin is administered intravenously to the subject in a dose of about 75 mg/m² following administration of the pemetrexed on Day 1 of each of cycles 1 to 4. In certain embodiments, the cisplatin is administered intravenously to the subject in a dose of 75 mg/m² following administration of the pemetrexed on Day 1 of each of cycles 1 to 4.

In certain embodiments, the method comprises administering carboplatin to the subject.

In certain embodiments, the carboplatin is administered intravenously to the subject in a dose of about (target AUC)×[(140−age)×(weight in kg)/serum creatinine (mg/dL)×72(×0.85 if female)+25], wherein the target AUC is from AUC 2.5 to AUC 5.

In certain embodiments, the target AUC is AUC 5.

In certain embodiments, the carboplatin is administered intravenously to the subject in a dose of about (target AUC)×[(140−age)×(weight in kg)/serum creatinine (mg/dL)×72(×0.85 if female)+25] following administration of the pemetrexed on Day 1 of each of cycles 1 to 4, wherein the target AUC is 5 mg*min/mL and the dose of carboplatin per administration is not to exceed 750 mg.

In certain embodiments, the carboplatin is administered intravenously to the subject in a dose of (target AUC)×[(140−age)×(weight in kg)/serum creatinine (mg/dL)×72(×0.85 if female)+25] following administration of the pemetrexed on Day 1 of each of cycles 1 to 4, wherein the target AUC is 5 mg*min/mL and the dose of carboplatin per administration is not to exceed 750 mg.

In certain embodiments, the ADC is tusamitamab and the anti-PD-1 antibody is pembrolizumab.

### EXAMPLES

### Example 1. Phase 2, Open-Label, Multicenter Trial of Combination Treatment of Patients with CEACAM5-Positive Expression Advanced/Metastatic NSQ NSCLC

The efficacy, safety, and pharmacokinetic (PK) of tusamitamab ravtansine, an ADC consisting of anti-carcinoembryonic antigen-related cell adhesion molecule 5 (CEACAM5) conjugated to the cytotoxic agent DM4 is assessed in human subjects with in CEACAM5-positive advanced/metastatic non-squamous non-small-cell lung cancer.

In a previous Phase 1 trial (NCT02187848), 92 NSQ NSCLC patients treated with tusamitamab ravtansine showed anti-tumor activity in 64 heavily pre-treated patients with NSQ NSCLC with tumor CEACAM5 expression ≥50%. This anti-tumor activity was associated with an overall response rate of 20.3% per Response Evaluation Criteria in Solid Tumors (RECIST)∘1.1 (95%. CI: 12.27%-31.71%), warranting further development of tusamitamab ravtansine to treat this patient population. In 28 heavily pre-treated patients with NSQ NSCLC with tumor CEACAM5 expression ≥1% and <5 0%, the overall response rate was 7.1% per RECIST∘1.1 (95%. CI: 1.98%-22.65%).

Pembrolizumab is the first approved and the most used immune checkpoint inhibitor (ICI) in the first line NSCLC in combination with standard of care (SOC) and as a single agent therapy (see, e.g., Gandhi, L., et al., N. Engl. J. Med. 2018, 378 (22): 2078-926; and Tony, S K M, et al., Lancet 2019, 393 (10183): 1819-30, which are incorporated herein by reference in their entirety). The combination of tusamitamab ravtansine with an ICI should lead to improved outcomes, as compared to a combination with untargeted, toxic systemic chemotherapy.

### Objectives

### Primary Objective

The primary objective of this trial is to assess the tolerability and to determine recommended doses of tusamitamab ravtansine in combination with pembrolizumab and tusamitamab ravtansine in combination with pembrolizumab and platinum-based chemotherapy with or without pemetrexed, in the NSQ NSCLC population. The endpoint consists of the incidence of drug-related dose-limiting toxicity (DLT) at Cycle 1 (C1D1 to C1D21), including but not limited to corneal toxicity.

### Secondary Objective

The secondary objectives of this trial are the following:
(i) To assess the safety and tolerability of tusamitamab ravtansine in combination with pembrolizumab (T2), and tusamitamab ravtansine in combination with pembrolizumab and platinum-based chemotherapy (T3) with or without pemetrexed (T4). The endpoint consists of the incidence of treatment-emergent adverse events (TEAEs), serious adverse events (SAEs) and laboratory abnormalities according to the National Cancer Institute (NCI) Common Terminology Criteria for Adverse Events (CTCAE) V5.0.
(ii) To assess the antitumor activity of tusamitamab ravtansine in combination with pembrolizumab, and tusamitamab ravtansine in combination with pembrolizumab and platinum-based chemotherapy with or without pemetrexed in the NSQ NSCLC population. The endpoint consists of an objective response rate defined as proportion of participants who have a confirmed complete response (CR) or partial response (PR) as per RECIST 1.1.
(iii) To assess the pharmacokinetics (PK) of tusamitamab ravtansine, pembrolizumab, pemetrexed, cisplatin, and carboplatin, each when given in combination as a doublet (tusamitamab ravtansine+pembrolizumab-T2) or a triplet (tusamitamab ravtansine+pembrolizumab+platinum-based chemotherapy-T3) or a quadruplet (tusamitamab ravtansine+pembrolizumab+platinum-based chemotherapy+pemetrexed-T4). The endpoint consists of pharmacokinetic concentrations of tusamitamab ravtansine, pembrolizumab, pemetrexed, cisplatin, and carboplatin.
(iv) To assess the immunogenicity of tusamitamab ravtansine in combination with pembrolizumab, and tusamitamab ravtansine in combination with pembrolizumab and platinum based chemotherapy with or without pemetrexed. The endpoint consists of incidence of anti-therapeutic antibodies (ATAs) against tusamitamab ravtansine.

### Study Design

This study is a Phase 2, open-label, multicenter study comprised of 3 parts:

Part A is to assess safety, efficacy (anti-tumor activity), and PK of tusamitamab ravtansine combined with pembrolizumab (T2) in NSQ NSCLC participants with CEACAM5 high expression tumors (defined as CEACAM5 immunohistochemistry [IHC] intensity ≥2+ in ≥50% of tumor cells. Intensity of the CEACAM5 staining is scored on a scale of 0 to 3, where 0 is negative, 1+ is weak positive, 2+ is moderate positive, and 3+ is strong positive. A general description of IHC process including scoring is given by So-Woon Kim et al., J Pathol Transl Med, 2016, 50 (6): 411-418, doi: 10.4132/jptm.2016.08.08, which is incorporated herein by reference in its entirety.

Part B is to assess safety, efficacy (anti-tumor activity), and PK of tusamitamab ravtansine combined with pembrolizumab and platinum-based chemotherapy (T3) in NSQ NSCLC participants with CEACAM5 high expression tumors; and

Part C is to assess safety, efficacy (anti-tumor activity), and PK of tusamitamab ravtansine combined with pembrolizumab, platinum-based chemotherapy and pemetrexed (T4) in NSQ NSCLC participants with CEACAM5 high or moderate expression (defined as intensity ≥2+ in ≥1% and <50% of tumor cells) tumors.

During the prescreening phase, patients' tumor samples are collected to evaluate CEACAM5 status (central assessment by IHC). The central assessment by immunohistochemistry (IHC) is based on the intensity evaluation of the staining of CEACAM5 to evaluate antigen expression.

During the screening phase, only participants with NSQ NSCLC determined to be CEACAM5 high expression (≥50%) tumors go through protocol screening procedures and are enrolled in Part A, Part B or Part C per Investigator's choice. Participants with CEACAM5 moderate expression (≥1% and <50%) tumors go through protocol screening procedures and are enrolled in Part C.

### Part A

The tolerability of the tusamitamab ravtansine and pembrolizumab combination (T2) is assessed.

The first 3 participants receive once every three weeks (Q3W) a 200 mg pembrolizumab infusion followed by a tusamitamab ravtansine infusion at the starting dose of 150 mg/m².

The DLT observation period is the first cycle (21 days). Depending on the DLTs observed, up to 3 dose levels (DLs) of tusamitamab ravtansine are tested: 150 mg/m², 170 mg/m², and 120 mg/m².

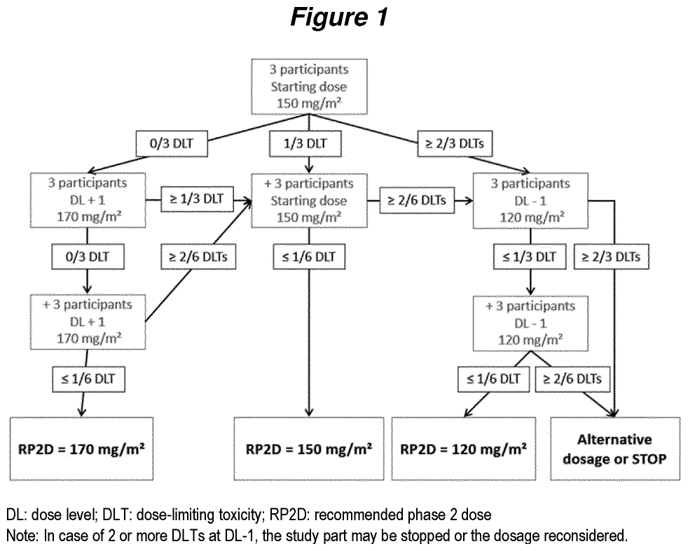

For each DL of the combination arm (starting dose, DL plus 1 [DL+1], and DL minus 1 [DL-1] if applicable), a minimum of 1 week is mandatory between the first dose of the first participant treated at this DL and the first dose of the next participant treated at the same DL. Once 3 participants have been treated at this DL and are DLT-evaluable, the tolerability of the combination is assessed according to the algorithm illustrated in FIG. 1.

### Part B

The tolerability and safety of the pembrolizumab, tusamitamab ravtansine, and platinum-based chemotherapy combinations (T3) are assessed. Participants can be assigned to either cisplatin or carboplatin, per Investigator choice.

Cisplatin combination arm: Participants receive Q3W pembrolizumab 200 mg+tusamitamab ravtansine (150 mg/m², 170 mg/m², or 120 mg/m²)+cisplatin 75 mg/m², all on Day 1 for the first 4 cycles, followed by Q3W pembrolizumab 200 mg+tusamitamab ravtansine (150 mg/m², 170 mg/m², or 120 mg/m²) on Day 1 of subsequent cycles.

Carboplatin combination arm: Participants receive Q3W pembrolizumab 200 mg+tusamitamab ravtansine (150 mg/m², 170 mg/m², or 120 mg/m²)+carboplatin AUC 5, all on Day 1 for the first 4 cycles, followed by Q3W pembrolizumab 200 mg+tusamitamab ravtansine (150 mg/m², 170 mg/m² or 120 mg/m² Q3W) on Day 1 of subsequent cycles.

The DLT observation period is the first cycle (21 days). Depending on the DLTs observed, up to 3 dose levels (DLs) of tusamitamab ravtansine can be tested during this safety run-in part: 150 mg/m², 170 mg/m², and 120 mg/m².

For each DL of the cisplatin and carboplatin combination arms, a minimum of 1 week is mandatory between the first dose of the first participant treated at this DL and the first dose of the next participant treated at the same DL. Once 3 participants assigned to a combination arm have been treated at a DL and are DLT-evaluable, the tolerability of the combination is assessed according to the decision algorithm as illustrated in FIG. 1. The tolerability of each triplet combination is assessed in at least 6 participants.

Approximately 12 to 36 treated participants in the cisplatin and carboplatin combination arms in Part B (6 to 18 in each arm) are expected to be evaluable for tolerability and safety.

### Part C

The tolerability and safety of the tusamitamab ravtansine, pembrolizumab, platinum-based chemotherapy and pemetrexed combinations (T4) are assessed. Participants can be assigned to either cisplatin or carboplatin, per Investigator choice.

Cisplatin combination arm: Participants receive Q3W pembrolizumab 200 mg+tusamitamab ravtansine (150 mg/m², 170 mg/m², or 120 mg/m²)+pemetrexed 500 mg/m² (with vitamin supplementation)+cisplatin 75 mg/m² all on Day 1 for the first 4 cycles, followed by Q3W pembrolizumab 200 mg+tusamitamab ravtansine (150 mg/m², 170 mg/m², or 120 mg/m²)+pemetrexed 500 mg/m² (with vitamin supplementation) on Day 1 of subsequent cycles.

Carboplatin combination arm: Participants receive Q3W pembrolizumab 200 mg+tusamitamab ravtansine (150 mg/m², 170 mg/m², or 120 mg/m²)+pemetrexed 500 mg/m² (with vitamin supplementation)+carboplatin AUC 5 all on Day 1 for the first 4 cycles, followed by Q3W pembrolizumab 200 mg+tusamitamab ravtansine (150 mg/m², 170 mg/m² or 120 mg/m² Q3W)+pemetrexed 500 mg/m² (with vitamin supplementation) on Day 1 of subsequent cycles.

The DLT observation period is the first cycle (21 days). Depending on the DLTs observed, up to 3 dose levels (DLs) of tusamitamab ravtansine can be tested during this safety run-in part: 150 mg/m², 170 mg/m², and 120 mg/m².

For each DL of the cisplatin and carboplatin combination arms, a minimum of 1 week is mandatory between the first dose of the first participant treated at this DL and the first dose of the next participant treated at the same DL. Once 3 participants assigned to a combination arm have been treated at a DL and are DLT-evaluable, the tolerability of the combination is assessed according to the decision algorithm illustrated in FIG. 1. The tolerability of each quadruplet combination is assessed in at least 6 participants.

Approximately 12 to 36 treated participants in the cisplatin and carboplatin combination arms in Part C (6 to 18 in each arm) are expected to be evaluable for tolerability and safety.

### Patients

### Inclusion Criteria

Participants are eligible to be included in the study if they satisfy the following criteria:

Histologically- or cytologically-confirmed diagnosis of advanced or metastatic NSQ NSCLC with no epidermal growth factor receptor (EGFR) sensitizing mutation or v-raf murine sarcoma viral oncogene homolog B1 (BRAF) mutation or anaplastic large-cell lymphoma kinase (ALK)/c-ros oncogene (ROS) alterations.

No prior systemic chemotherapy for the treatment of the participant's advanced or metastatic disease (treatment with chemotherapy and/or radiation as part of neoadjuvant/adjuvant therapy is allowed as long as completed at least 6 months prior to diagnosis of advanced or metastatic disease).

Expression of CEACAM5 as demonstrated prospectively by a centrally assessed IHC assay of ≥2+ in intensity involving at least 50% (for Part A and Part B) and at least 1% (for Part C) of the tumor cell population in archival tumor sample (or if not available fresh biopsy sample will be collected if considered an acceptable risk by the treating physician). At least 5 slides of formalin-fixed, paraffin embedded (FFPE) tumor tissue sectioned at a thickness of 4 μm are required. If less material is available, the patient can still be considered eligible after discussion with the Sponsor, who may assess and confirm that the available material is sufficient for key evaluations.

Measurable disease based on RECIST 1.1.

### Exclusion Criteria

Participants are excluded from the study if any of the following criteria applied:

### 1. Medical Conditions

Medical condition requiring concomitant administration of a medication with a narrow therapeutic window, that is metabolized by cytochrome P450 (CYP450), and for which a dose reduction cannot be considered.

Medical conditions requiring concomitant administration of strong cytochrome P450 family 3 subfamily A (CYP3A) inhibitor, unless it could be discontinued at least 2 weeks before the first administration of study intervention and for the entire study treatment period.

Uncontrolled brain metastases and history of leptomeningeal disease.

Significant concomitant illness, including any severe medical condition that, in the opinion of the investigator or Sponsor, would impair the patient's participation in the study or interpretation of the results.

History within the last 3 years of an invasive malignancy other than the one treated in this study, with the exception of resected/ablated basal or squamous-cell carcinoma of the skin or carcinoma in situ of the cervix, or other local tumors considered cured by local treatment.

History of known acquired immunodeficiency syndrome (AIDS) related illnesses or known HIV disease requiring antiretroviral treatment, or active hepatitis A, B (defined as either positive hepatitis B surface antigen (HBsAg) or positive hepatitis B viral DNA test above the lower limit of detection of the assay), or C (defined as a known positive hepatitis C antibody result and known quantitative hepatitis C virus (HCV) RNA results greater than the lower limits of detection of the assay) infection. HIV serology will be tested at screening only for participants enrolled at German sites or in any country where mandatory per local requirements.

History of active autoimmune disease that has required systemic treatment in the past 2 years.

History of allogeneic tissue/solid organ transplantation.

Active infection requiring IV systemic therapy within 2 weeks prior to first study intervention administration or active tuberculosis.

Interstitial lung disease or history of pneumonitis that has required oral or IV steroids.

Non-resolution of any prior treatment-related toxicity to <Grade 2 according to NCI CTCAE V5.0, with the exception of alopecia, vitiligo, or active thyroiditis controlled with hormone-replacement therapy.

Unresolved corneal disorder or any previous corneal disorder considered by an ophthalmologist to predict higher risk of drug-induced keratopathy. The use of contact lenses is not permitted. Patients using contact lenses who are not willing to stop wearing them for the duration of the study intervention are excluded.

Symptomatic herpes zoster within 3 months prior to screening.

Significant allergies to humanized monoclonal antibodies.

Clinically significant multiple or severe drug allergies, intolerance to topical corticosteroids, or severe post-treatment hypersensitivity reactions (including, but not limited to, erythema multiforme major, linear immunoglobulin A [IgA] dermatosis, toxic epidermal necrolysis, and exfoliative dermatitis).

### 2. Prior/Concomitant Therapy

Concurrent treatment with any other anticancer therapy.

Have received prior chemotherapy treatment for advanced/metastatic NSCLC.

The patient is a candidate for a curative treatment with either surgical resection and/or chemoradiation.

Washout period before the first administration of study intervention of less than 3 weeks or less than 5 times the half-life, whichever is shorter, for any investigational treatment.

Any prior therapy targeting CEACAM5.

Any prior treatment with any other anti-programmed cell death protein 1 (PD-1), or programmed death-ligand 1 (PD-L1) or programmed death-ligand 2 (PD-L2), anti-CD137, or anti-cytotoxic T-lymphocyte-associated antigen-4 (CTLA-4) antibody (including ipilimumab or any other antibody or drug specifically targeting T-cell co-stimulation or checkpoint pathways).

Any prior maytansinoid treatment (maytansinoid emtansine (DM1) or ravtansine (DM4) ADC).

Is receiving systemic steroid therapy ≤3 days prior to the first dose of study therapy or receiving any other form of immunosuppressive medication. Daily steroid replacement therapy or any corticosteroid premedication if applicable are allowed.

Any radiation therapy to lung >30 Gy within 6 months of first study intervention administration.

Has received or will receive a live vaccine within 30 days prior to the first study intervention administration.

Any major surgery within the preceding 3 weeks of the first study intervention administration.

Current participation in any other clinical study involving an investigational study treatment or any other type of medical research.

Poor organ function as defined by any one of the following:
Serum creatinine >1.5×upper limit of normal (ULN) or 1.0-1.5×ULN with estimated glomerular filtration rate (eGFR)<60 mL/min/1.73 m² as estimated using a modification of diet in renal disease (MDRD) formula.

Total bilirubin >1.0×ULN.

Aspartate aminotransferase (AST), alanine aminotransferase (ALT) >2.5×ULN or AST, ALT >1.5×ULN concomitant with ALP >2.5×ULN. ALP >5×ULN with normal ALT/AST, for patients with bone metastases.

Neutrophils <1.5×10⁹/L or platelet count <100×10⁹/L or hemoglobulin <9 g/dL (no blood infusion within 2 weeks before screening)

Thyroid-stimulating hormone (TSH) out of normal limits. If TSH is not within normal limits at baseline, the subject may still be eligible if T3 and free T4 are within the normal limits

International normalized ratio (INR) >1.5 unless participant is receiving anticoagulant therapy or within therapeutic range if receiving anticoagulation that would affect the INR

### Target Number

In Part A, approximately 38 to 150 participants are prescreened (prescreening failure estimated to be about 80%, and study screen failure rate to be about 20%) to achieve 6 up to 24 DLT-evaluable participants (including 12 DLT-evaluable participants at the recommended Phase 2 dose (RP2D), in addition to 12 participants treated at DL other than RP2D).

In Part B, approximately 75 to 225 participants are prescreened (prescreening failure estimated to be about 80% and study screen failure rate to be about 20%) to achieve 12 up to 36 DLT evaluable participants (6 to 18 DLT evaluable participants in each triplet combination arm).

In Part C, approximately 28 to 82 participants are prescreened (prescreening failure estimated to be 45% and study screen failure rate to be 20%) to achieve 12 up to 36 DLT evaluable participants (6 to 18 DLT evaluable participants in each quadruplet combination arm).

“Enrolled” means a participant's, or their legally acceptable representative's, agreement to participate in a clinical study following completion of the informed consent process. Potential participants who are screened for the purpose of determining eligibility for the study, but do not participate in the study, are not considered enrolled, unless otherwise specified by the protocol.

### Clinical and Laboratory Monitoring

### Statistical Analysis

Efficacy analyses (overall response rate (ORR) as per RECIST 1.1) are performed on the all-treated population by dose level and overall, for each combination arm. Objective response rates are derived using the local radiologist's/Investigator's assessment.

The study cut-off for primary safety endpoints analysis (DLT) is at the end of the first cycle of the last participant treated to determine the RP2D in Part A, Part B or Part C. The study cut-off for ORR (secondary efficacy endpoint analysis) corresponds to the date on which all treated participants have had at least 2 post-baseline tumor assessments, experienced confirmed objective response, or have discontinued the study for any reason. This study cut-off occurs approximately 4.5 months after the date of the first investigational medicinal product (IMP) administration of the last participant: 3 months for 2 tumor assessments and 1.5 months if a confirmation of response is needed. All analyses are updated at that time.

All safety analyses are performed on the all-treated population, by treatment arm, by DL (if applicable) and overall. For each safety parameter, a baseline value is defined as the latest value or measurement taken up to the first administration of the IMP.

### Pharmacokinetics (PK)

Blood samples are collected for the measurement of tusamitamab ravtansine, pembrolizumab, cisplatin or carboplatin and pemetrexed concentrations. The actual date and time of each sample are recorded.

Concentrations of tusamitamab ravtansine, pemetrexed and cisplatin or carboplatin are used for population PK analysis by nonlinear mixed-effects modeling. Empirical Bayesian estimation of individual exposure parameters such as maximum concentration (Cₘₐₓ), trough concentration (C_{trough}) and area under the curve (AUC) are derived.

Pembrolizumab C_{trough} values for Part A, Part B and Part C are reported.

### Efficacy Assessments

### 1. Measurability of Tumor at Baseline

At baseline, tumor lesions/lymph nodes are categorized measurable or non-measurable as follows.

Measurable lesions must be accurately measured in at least 1 dimension (longest diameter in the plane of the measurement to be recorded) with a minimum size of:

10 mm by CT scan (CT scan slice thickness no greater than 5 mm).

10 mm caliper measurement by clinical exam (lesions which cannot be accurately measured with calipers should be recorded as non-measurable).

20 mm by chest X-ray.

Malignant lymph nodes: To be considered pathologically enlarged and measurable, a lymph node must be ≥15 mm in short axis when assessed by computerized tomography scan (CT scan) (CT scan slice thickness recommended to be no greater than 5 mm). At baseline and in follow-up, only the short axis will be measured and followed.

Non-measurable lesions are all other lesions, including small lesions (longest diameter <10 mm or pathological lymph nodes with ≥10 to <15 mm short axis), as well as truly non-measurable lesions. Lesions considered non-measurable include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

Special considerations regarding lesion measurability are presented below.

### (1) Bone Lesions:

Bone scan, positron emission tomography scan or plain films are not considered adequate imaging techniques to measure bone lesions. However, these techniques could be used to confirm the presence or disappearance of bone lesions.

Lytic bone lesions or mixed lytic-blastic lesions, with identifiable soft tissue components, that could be evaluated by cross sectional imaging techniques such as CT scan or magnetic resonance imaging scan (MRI scan) could be considered as measurable lesions if the soft tissue component meets the definition of measurability described above.

Blastic bone lesions are non-measurable.

### (2) Cystic Lesions:

Lesions that meet the criteria for radiographically defined simple cysts should not be considered as malignant lesions (neither measurable nor non-measurable) since they are, by definition, simple cysts.

‘Cystic lesions’ thought to represent cystic metastases could be considered as measurable lesions, if they meet the definition of measurability described above. However, if non-cystic lesions are present in the same patient, these are preferred for selection as target lesions.

(3) Lesions with Prior Local Treatment:

Tumor lesions situated in a previously irradiated area, or in an area subjected to other loco-regional therapy, are usually not considered measurable unless there has been demonstrated progression in the lesion.

### 2. Method of Assessment

Measurements are recorded in metric notation, using calipers if clinically assessed. Baseline evaluations are performed as close as possible to the treatment start and never more than 4 weeks before the beginning of the treatment.

The same method of assessment and the same technique are used to characterize each identified and reported lesion at baseline and during follow-up. Imaging-based evaluation is performed rather than clinical examination unless the lesion(s) being followed cannot be imaged but are assessable by clinical examination.

Clinical lesions: Clinical lesions are only considered measurable when they are superficial and ≥10 mm diameter as assessed using calipers.

Chest X-ray: Chest CT is preferred over chest X-ray, particularly when progression is an important endpoint, since CT is more sensitive than X-ray, particularly in identifying new lesions. However, lesions on chest X-ray may be considered measurable if they are clearly defined and surrounded by aerated lung.

CT, MRI: CT is the best currently available and reproducible method to measure lesions selected for response assessment. Measurability of lesions on CT scan is based on the assumption that CT slice thickness is 5 mm or less. When CT scans have slice thickness greater than 5 mm, the minimum size for a measurable lesion is twice the slice thickness.

Tumor markers: Tumor markers alone can not be used to assess objective tumor response.

Cytology, histology: These techniques could be used to differentiate between partial response (PR) and complete response (CR) in rare cases if required by protocol.

### 3. Baseline Documentation of ‘Target’ and ‘Non-Target’ Lesions

When more than 1 measurable lesion is present at baseline all lesions up to a maximum of 5 lesions total (and a maximum of 2 lesions per organ) representative of all involved organs are identified as target lesions and are recorded and measured at baseline.

Target lesions are selected on the basis of their size (lesions with the longest diameter), be representative of all involved organs, and lent themselves to reproducible repeated measurements.

Lymph nodes are normal anatomical structures which may be visible by imaging even if not involved by tumor. Pathological nodes which are defined as measurable and may be identified as target lesions must meet the criterion of a short axis of ≥15 mm by CT scan. Only the short axis of these nodes contributed to the baseline sum. All other pathological nodes (those with short axis ≥10 mm but <15 mm) are not considered non-target lesions. Nodes that have a short axis <10 mm are considered non-pathological and are not recorded or followed.

A sum of the diameters (longest for non-nodal lesions, short axis for nodal lesions) for all target lesions is calculated and reported as the baseline sum diameters. The baseline sum diameters are used as reference to further characterize any objective tumor regression in the measurable dimension of the disease.

All other lesions (or sites of disease) including pathological lymph nodes are identified as non-target lesions and were also recorded at baseline. Measurements are not required and these lesions are followed as ‘present’, ‘absent’, or ‘unequivocal progression’. In addition, it is possible to record multiple non-target lesions involving the same organ as a single item on the case (e.g., ‘multiple enlarged pelvic lymph nodes’ or ‘multiple liver metastases’).

### 4. Response Criteria

Response criteria are described in Table 1.

TABLE 1
Response criteria

| Response | |
|---|---|
| criterion | Evaluation of target lesions |
| CR | Disappearance of all target lesions. Any pathological |
| | lymph nodes (whether target or non-target) must have |
| | reduction in short axis to <10 mm. |
| PR | At least a 30% decrease in the sum of diameters of target |
| | lesions, taking as reference the baseline sum diameters. |
| PD | At least a 20% increase in the sum of diameters of target |
| | lesions, taking as reference the smallest sum on study |
| | (this includes the baseline sum if that is the smallest |
| | on study). In addition to the relative increase of 20%, |
| | the sum must also demonstrate an absolute increase of at |
| | least 5 mm. (Note: the appearance of 1 or more new lesions |
| | is also considered progression). |
| SD | Neither sufficient shrinkage from the baseline study to |
| | qualify for PR nor sufficient increase to qualify for PD, |
| | taking as reference the smallest sum diameters while on |
| | study. |

Abbreviations: CR = complete response; PD = progressive disease; PR = partial response; SD = stable disease.

### 5. Special Notes on the Assessment of Target Lesions

Lymph nodes identified as target lesions have the actual short axis measurement recorded and are measured in the same anatomical plane as the baseline examination, even if the nodes regress to below 10 mm on study. This means that when lymph nodes are included as target lesions, the ‘sum’ of lesions is not zero even if CR criteria is met, since a normal lymph node is defined as having a short axis of <10 mm. For PR, SD and PD, the actual short axis measurement of the nodes is included in the sum of target lesions.

Target lesions that become ‘too small to measure’: All lesions (nodal and non-nodal) recorded at baseline have their actual measurements recorded at each subsequent evaluation, even when very small (e.g., 2 mm). However, sometimes lesions or lymph nodes which are recorded as target lesions at baseline became so faint on CT scan that the radiologist may not feel comfortable assigning an exact measure and may report them as being ‘too small to measure’. When this occurs, it is important that a value be recorded on the case report form (CRF). If it is the opinion of the radiologist that the lesion has likely disappeared, the measurement is recorded as 0 mm. If the lesion is believed to be present and is faintly seen but too small to measure, a default value of 5 mm is assigned.

When non-nodal lesions ‘fragment’, the longest diameters of the fragmented portions are added together to calculate the target lesion sum. Similarly, as lesions coalesce, a plane between them is maintained that aid in obtaining maximal diameter measurements of each individual lesion. If the lesions have truly coalesced such that they are no longer separable, the vector of the longest diameter in this instance is the maximal longest diameter for the ‘coalesced lesion’.

### 6. Evaluation of Non-Target Lesions

While some non-target lesions are actually measurable, they needed not be measured and instead are assessed only qualitatively at the time points specified in the protocol.

CR: Disappearance of all non-target lesions and normalization of tumor marker level. All lymph nodes must be non-pathological in size (<10 mm short axis).

Non-CR/Non-PD: Persistence of 1 or more non-target lesion(s) and/or maintenance of tumor marker level above the normal limits.

Progressive Disease (PD): Unequivocal progression of existing non-target lesions. (Note: the appearance of 1 or more new lesions is also considered progression).

The concept of progression of non-target disease is as follows:

When the participant also has measurable disease; in this setting, to achieve ‘unequivocal progression’ on the basis of the non-target disease, there needs to be an overall level of substantial worsening in non-target disease such that, even in presence of SD or PR in target disease, the overall tumor burden has increased sufficiently to merit discontinuation of therapy. A modest ‘increase’ in the size of 1 or more non-target lesions is usually not sufficient to qualify for unequivocal progression status.

When the participant has only non-measurable disease; to achieve ‘unequivocal progression’ on the basis of the non-target disease, there needs to be an overall level of substantial worsening such that the overall tumor burden has increased sufficiently to merit discontinuation of therapy. A modest ‘increase’ in the size of 1 or more non-target lesions is usually not sufficient to qualify for unequivocal progression status. Because worsening in non-target disease cannot be easily quantified (by definition: if all lesions are truly non-measurable) a useful test that can be applied when assessing patients for unequivocal progression is to consider if the increase in overall disease burden based on the change in non-measurable disease is comparable in magnitude to the increase that would be required to declare PD for measurable disease: i.e., an increase in tumor burden representing an additional 73% increase in ‘volume’ (which is equivalent to a 20% increase diameter in a measurable lesion). Examples include an increase in a pleural effusion from ‘trace’ to ‘large’, an increase in lymphangitic disease from localized to widespread, or may be described in protocols as ‘sufficient to require a change in therapy’. If ‘unequivocal progression’ is seen, the patient is considered to have overall PD at that point.

### 7. New Lesions

The appearance of new malignant lesions denotes disease progression. The finding of a new lesion is unequivocal: i.e., not attributable to differences in scanning technique, change in imaging modality or findings thought to represent something other than tumor (for example, some ‘new’ bone lesions may be simply healing or flare of preexisting lesions). This is particularly important when the participant's baseline lesions show PR or CR. For example, necrosis of a liver lesion is reported on a CT scan report as a ‘new’ cystic lesion, which it is not.

A lesion identified on a follow-up study in an anatomical location that is not scanned at baseline is considered a new lesion and indicates disease progression. An example of this is a patient who has visceral disease at baseline and while on study has a CT or MRI brain ordered which reveals metastases. The participant's brain metastases are considered to constitute PD even if he/she did not have brain imaging at baseline.

If a new lesion is equivocal, for example because of its small size, continued therapy and follow-up evaluation clarify if it represents new disease. If repeated scans confirm that there is a new lesion, then progression is declared using the date of the initial scan.

While fluorodeoxyglucose-positron emission tomography (FDG-PET) response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:

Negative FDG-PET at baseline, with a positive FDG-PET at follow-up was a sign of PD based on a new lesion.

No FDG-PET at baseline and a positive FDG-PET at follow-up:

If the positive FDG-PET at follow-up corresponds to a new site of disease confirmed by CT, this is PD;

If the positive FDG-PET at follow-up is not confirmed as a new site of disease on CT, additional follow-up CT scans are needed to determine if there is truly progression occurring at that site (if so, the date of PD is the date of the initial abnormal FDG-PET scan). If the positive FDG-PET at follow-up corresponds to a preexisting site of disease on CT that was not progressing on the basis of the anatomic images, this is not PD.

### 8. Evaluation of Best Overall Response

Time point response: At each protocol specified time point, a response assessment occurs. Table 2 provides a summary of the overall response status calculation at each time point for patients who have measurable disease at baseline.

TABLE 2
Response in patients with target disease

| Target | | | Overall |
|---|---|---|---|
| lesions | Non-target lesions | New lesions | response |
| CR | CR | No | CR |
| CR | Non-CR/non-PD | No | PR |
| CR | Not evaluated | No | PR |
| PR | Non-PD or not | No | PR |
| | all evaluated | | |
| SD | Non-PD or not | No | SD |
| | all evaluated | | |
| Not all | Non-PD | No | Inevaluable |
| evaluated | | | |
| PD | Any | Yes or No | PD |
| Any | PD | Yes or No | PD |
| Any | Any | Yes | PD |

Abbreviations: CR = complete response; PD = progressive disease; PR = partial response; SD = stable disease.

When patients have non-measurable (therefore non-target) disease only, Table 3 is to be used.

TABLE 3
Response in patients with non-target disease only

| Non-target lesions | New lesions | Overall response |
|---|---|---|
| CR | No | CR |
| Non-CR/non-PD | No | Non-CR/non-PD |
| Not all evaluated | No | Inevaluable |
| Unequivocal PD | Yes or No | PD |
| Any | Yes | PD |

Abbreviations: CR = complete response; PD = progressive disease; PR = partial response; SD = stable disease.

Missing assessments and inevaluable designation: When no imaging/measurement is done at all at a particular time point, the patient is not evaluable (NE) at that time point.

If only a subset of lesion measurements are made at an assessment, usually the case is also considered NE at that time point, unless a convincing argument can be made that the contribution of the individual missing lesion(s) would not change the assigned time point response. This would be most likely to happen in the case of PD.

### 9. Special Notes on Response Assessment

When nodal disease is included in the sum of target lesions and the nodes decrease to ‘normal’ size (<10 mm), they may still have a measurement reported on scans. This measurement is recorded even though the nodes are normal in order not to overstate progression should it be based on increase in size of the nodes. As noted earlier, this means that patients with CR may not have a total sum of ‘zero’ on the CRF.

In trials where confirmation of response is required, repeated ‘NE’ time point assessments may have complicated best response determination. The analysis plan for the trial addresses how missing data/assessments are addressed in determination of response and progression. For example, in most trials it is reasonable to consider a patient with time point responses of PR-NE-PR as a confirmed response.

Patients with a global deterioration of health status requiring discontinuation of treatment without objective evidence of disease progression at that time are reported as ‘symptomatic deterioration’. Every effort is made to document objective progression even after discontinuation of treatment. Symptomatic deterioration is not a descriptor of an objective response; it is a reason for stopping study therapy.

The objective response status of such patients is to be determined by evaluation of target and non-target disease. For equivocal findings of progression (e.g., very small and uncertain new lesions; cystic changes or necrosis in existing lesions), treatment may continue until the next scheduled assessment. If at the next scheduled assessment, progression was confirmed, the date of progression is the earlier date when progression was suspected.

### 10. Duration of Response

The duration of overall response is measured from the time measurement criteria are first met for CR/PR (whichever is first recorded) until the first date that recurrent or PD is objectively documented (taking as reference for PD the smallest measurements recorded on study).

The duration of overall CR is measured from the time measurement criteria are first met for CR until the first date that recurrent disease is objectively documented.

### 11. Duration of Stable Disease

Stable disease is measured from the start of the treatment until the criteria for progression are met, taking as reference the smallest sum on study (if the baseline sum was the smallest, this is the reference for calculation of PD).

Efficacy measurements are performed based on best practice such as the practice described by Eisenhauer, E. A. et al., “New response evaluation criteria in solid tumours: Revised RECIST guideline (version 1.1),” Eur. J. Cancer, 2009, 45:228-47, which is incorporated herein by reference in its entirety.

### Results

The Table 4 below shows the summary of best overall response and objective response rates as per RECIST 1.1 with confirmation of response by investigator.

TABLE 4

| | T2 | | T3 | | T4 | | |
|---|---|---|---|---|---|---|---|
| | SAR408701 | SAR408701 | SAR408701 | SAR408701 | SAR408701 | SAR408701 | |
| Best Overall | 150 mg/m² | 170 mg/m² | 150 mg/m² | 170 mg/m² | 150 mg/m² | 170 mg/m² | All |
| Response [n (%)] | (N = 3) | (N = 2) | (N = 4) | (N = 1) | (N = 7) | (N = 3) | (N = 20) |
| Number | 3 | 2 | 4 | 1 | 7 | 3 | 20 |
| Complete Response | 0 | 0 | 0 | 0 | 0 | 0 | 0 |
| Partial Response | 3 (100) | 0 | 2 (50.0) | 0 | 3 (42.9) | 2 (66.7) | 10 (50.0) |
| Stable Disease^{a} | 0 | 2 (100) | 2 (50.0) | 1 (100) | 2 (28.6) | 1 (33.3) | 8 (40.0) |
| Progressive Disease | 0 | 0 | 0 | 0 | 2 (28.6) | 0 | 2 (10.0) |
| Not evaluable^{b} | 0 | 0 | 0 | 0 | 0 | 0 | 0 |
| Objective Response | 3 (100) | 0 | 2 (50.0) | 0 | 3 (42.9) | 2 (66.7) | 10 (50.0) |
| Rate (confirmed | | | | | | | |
| CR and PR) | | | | | | | |
| [n (%)] | | | | | | | |
| 95% CI^{c} | 29.24 to | 0.00 to | 6.76 to | 0.00 to | 9.90 to | 9.43 to | 27.20 to |
| | 100.00 | 84.19 | 93.24 | 97.50 | 81.59 | 99.16 | 72.80 |
| Disease control | 3 (100) | 2 (100) | 4 (100) | 1 (100) | 5 (71.4) | 3 (100) | 18 (90.0) |
| rate (confirmed | | | | | | | |
| CR or PR, SD) [n (%)] | | | | | | | |
| 95% CI^{c} | 29.24 to | 15.81 to | 39.76 to | 2.50 to | 29.04 to | 29.24 to | 68.30 to |
| | 100.00 | 100.00 | 100.00 | 100.00 | 96.33 | 100.00 | 98.77 |

CI: Confidence interval,
CR: Complete response,
PR: Partial response,
SD: Stable disease
^{a}Including participants with unconfirmed CR or PR.
^{b}Including participants with no post-baseline evaluable tumor assessment but with an early clinical progression.
^{c}Estimated by Clopper Pearson interval.

Confirmation of response (CR/PR) is required: the subsequent tumor assessment used for the confirmation must be assessed at least 4 weeks (>=28 days) after the initial CR/PR assessment. A response can be confirmed if there is a single non-evaluable tumor assessment performed in-between the two tumor assessments showing a response

The Table 5 below shows the summary of best overall response and objective response rate as per RECIST 1.1 with confirmation of response by investigator by PD-L1 expression among T2 part-Activity population.

TABLE 5

| | PD-L1 expression | | | | | | |
|---|---|---|---|---|---|---|---|
| | <1% | 1-49% | | ≥50% | | All | |
| | (N = 0) | (N = 2) | | (N = 3) | | (N = 5) | |
| Best Overall | | | | | | | |
| Response [n (%)] | | | | | | | |
| Number | 0 | 2 | | 3 | | 5 | |
| Complete Response | 0 | 0 | | 0 | | 0 | |
| Partial Response | 0 | 2 | (100) | 1 | (33.3) | 3 | (60.0) |
| Stable Disease^{a} | 0 | 0 | | 2 | (66.7) | 2 | (40.0) |
| Progressive Disease | 0 | 0 | | 0 | | 0 | |
| Not evaluable^{b} | 0 | 0 | | 0 | | 0 | |
| Objective Response | 0 | 2 | (100) | 1 | (33.3) | 3 | (60.0) |
| Rate (confirmed CR | | | | | | | |
| and PR) [n (%)] | | | | | | | |
| 95% CI^{c} | NC to NC | 15.81 to | | 0.84 to | | 14.66 to | |
| | | 100.00 | | 90.57 | | 94.73 | |
| Disease control rate | 0 | 2 | (100) | 3 | (100) | 5 | (100) |
| (confirmed CR or | | | | | | | |
| PR, SD) [n (%)] | | | | | | | |

| | | | | |
|---|---|---|---|---|
| 95% CI^{c} | NC to NC | 15.81 to | 29.24 to | 47.82 to |
| | | 100.00 | 100.00 | 100.00 |

CI: Confidence interval,
CR: Complete response,
PR: Partial response,
SD: Stable disease,
NE: Not evaluable,
NC: Not Calculated
^{a}Including participants with unconfirmed CR or PR.
^{b}Including participants with no post-baseline evaluable tumor assessment but with an early clinical progression.
^{c}Estimated by Clopper Pearson interval.

Confirmation of response (CR/PR) is required: the subsequent tumor assessment used for the confirmation must be assessed at least 4 weeks (>=28 days) after the initial CR/PR assessment. A response can be confirmed if there is a single non-evaluable tumor assessment performed in-between the two tumor assessments showing a response

The Table 6 below shows the summary of best overall response and objective response rate as per RECIST 1.1 with confirmation of response by investigator by PD-L1 expression among T3 part-Activity population.

TABLE 6

| | PD-L1 expression | | | | | | |
|---|---|---|---|---|---|---|---|
| | <1% | 1-49% | | ≥50% | | All | |
| | (N = 0) | (N = 4) | | (N = 1) | | (N = 5) | |
| Best Overall | | | | | | | |
| Response [n (%)] | | | | | | | |
| Number | 0 | 4 | | 1 | | 5 | |
| Complete Response | 0 | 0 | | 0 | | 0 | |
| Partial Response | 0 | 2 | (50.0) | 0 | | 2 | (40.0) |
| Stable Disease^{a} | 0 | 2 | (50.0) | 1 | (100) | 3 | (60.0) |
| Progressive Disease | 0 | 0 | | 0 | | 0 | |
| Not evaluable^{b} | 0 | 0 | | 0 | | 0 | |
| Objective Response | 0 | 2 | (50.0) | 0 | | 2 | (40.0) |
| Rate (confirmed CR | | | | | | | |
| and PR) [n (%)] | | | | | | | |
| 95% CI^{c} | NC to NC | 6.76 to | | 0.00 to | | 5.27 to | |
| | | 93.24 | | 97.50 | | 85.34 | |
| Disease control rate | 0 | 4 | (100) | 1 | (100) | 5 | (100) |
| (confirmed CR or | | | | | | | |
| PR, SD) [n (%)] | | | | | | | |

| | | | | |
|---|---|---|---|---|
| 95% CI^{c} | NC to NC | 39.76 to | 2.50 to | 47.82 to |
| | | 100.00 | 100.00 | 100.00 |

CI: Confidence interval,
CR: Complete response,
PR: Partial response,
SD: Stable disease,
NE: Not evaluable,
NC: Not Calculated
^{a}Including participants with unconfirmed CR or PR.
^{b}Including participants with no post-baseline evaluable tumor assessment but with an early clinical progression.
^{c}Estimated by Clopper Pearson interval.

Confirmation of response (CR/PR) is required: the subsequent tumor assessment used for the confirmation must be assessed at least 4 weeks (>=28 days) after the initial CR/PR assessment. A response can be confirmed if there is a single non-evaluable tumor assessment performed in-between the two tumor assessments showing a response

The Table 7 below shows the summary of best overall response and objective response rate as per RECIST 1.1 with confirmation of response by investigator by PD-L1 and CEACAM5 expression among T4 part-Activity population.

TABLE 7

| | PD-L1 expression | | | | | | | |
|---|---|---|---|---|---|---|---|---|
| | <1% | | 1-49% | | ≥50% | | All | |
| | (N = 2) | | (N = 6) | | (N = 2) | | (N = 10) | |
| CEACAM5 | | | | | | | | |
| expression: 1-49% | | | | | | | | |
| Best Overall | | | | | | | | |
| Response [n (%)] | | | | | | | | |
| Number | 2 | | 3 | | 2 | | 7 | |
| Complete Response | 0 | | 0 | | 0 | | 0 | |
| Partial Response | 2 | (100) | 2 | (66.7) | 0 | | 4 | (57.1) |
| Stable Disease^{a} | 0 | | 0 | | 1 | (50.0) | 1 | (14.3) |
| Progressive Disease | 0 | | 1 | (33.3) | 1 | (50.0) | 2 | (28.6) |
| Not evaluable^{b} | 0 | | 0 | | 0 | | 0 | |
| Objective Response | 2 | (100) | 2 | (66.7) | 0 | | 4 | (57.1) |
| Rate (confirmed CR | | | | | | | | |
| and PR) [n (%)] | | | | | | | | |
| 95% CI^{c} | 15.81 to | | 9.43 to | | 0.00 to | | 18.41 to | |
| | 100.00 | | 99.16 | | 84.19 | | 90.10 | |
| Disease control rate | 2 | (100) | 2 | (66.7) | 1 | (50.0) | 5 | (71.4) |
| (confirmed CR or | | | | | | | | |
| PR, SD) [n (%)] | | | | | | | | |
| 95% CI^{c} | 15.81 to | | 9.43 to | | 1.26 to | | 29.04 to | |
| | 100.00 | | 99.16 | | 98.74 | | 96.33 | |
| CEACAM5 | | | | | | | | |
| expression: ≥50% | | | | | | | | |
| Best Overall | | | | | | | | |
| Response [n (%)] | | | | | | | | |
| Number | 0 | | 2 | | 0 | | 2 | |
| Complete Response | 0 | | 0 | | 0 | | 0 | |
| Partial Response | 0 | | 1 | (50.0) | 0 | | 1 | (50.0) |
| Stable Disease^{a} | 0 | | 1 | (50.0) | 0 | | 1 | (50.0) |
| Progressive Disease | 0 | | 0 | | 0 | | 0 | |
| Not evaluable^{b} | 0 | | 0 | | 0 | | 0 | |
| Objective Response | 0 | | 1 | (50.0) | 0 | | 1 | (50.0) |
| Rate (confirmed CR | | | | | | | | |
| and PR) [n (%)] | | | | | | | | |
| 95% CI^{c} | NC to NC | | 1.26 to | | NC to NC | | 1.26 to | |
| | | | 98.74 | | | | 98.74 | |
| Disease control rate | 0 | | 2 | (100) | 0 | | 2 | (100) |
| (confirmed CR or | | | | | | | | |
| PR, SD) [n (%)] | | | | | | | | |
| 95% CI^{c} | NC to NC | | 15.81 to | | NC to NC | | 15.81 to | |
| | | | 100.00 | | | | 100.00 | |
| Overall | | | | | | | | |
| Best Overall | | | | | | | | |
| Response [n (%)] | | | | | | | | |
| Number | 2 | | 6 | | 2 | | 10 | |
| Complete Response | 0 | | 0 | | 0 | | 0 | |
| Partial Response | 2 | (100) | 3 | (50.0) | 0 | | 5 | (50.0) |
| Stable Disease^{a} | 0 | | 2 | (33.3) | 1 | (50.0) | 3 | (30.0) |
| Progressive Disease | 0 | | 1 | (16.7) | 1 | (50.0) | 2 | (20.0) |
| Not evaluable^{b} | 0 | | 0 | | 0 | | 0 | |
| Objective Response | 2 | (100) | 3 | (50.0) | 0 | | 5 | (50.0) |
| Rate (confirmed CR | | | | | | | | |
| and PR) [n (%)] | | | | | | | | |
| 95% CI^{c} | 15.81 to | | 11.81 to | | 0.00 to | | 18.71 to | |
| | 100.00 | | 88.19 | | 84.19 | | 81.29 | |
| Disease control rate | 2 | (100) | 5 | (83.3) | 1 | (50.0) | 8 | (80.0) |
| (confirmed CR or | | | | | | | | |
| PR, SD) [n (%)] | | | | | | | | |

| | | | | |
|---|---|---|---|---|
| 95% CI^{c} | 15.81 to | 35.88 to | 1.26 to | 44.39 to |
| | 100.00 | 99.58 | 98.74 | 97.48 |

CI: Confidence interval,
CR: Complete response,
PR: Partial response,
SD: Stable disease,
NE: Not evaluable,
NC: Not Calculated
^{a}Including participants with unconfirmed CR or PR.
^{b}Including participants with no post-baseline evaluable tumor assessment but with an early clinical progression.
^{c}Estimated by Clopper Pearson interval.

Confirmation of response (CR/PR) is required: the subsequent tumor assessment used for the confirmation must be assessed at least 4 weeks (>=28 days) after the initial CR/PR assessment. A response can be confirmed if there is a single non-evaluable tumor assessment performed in-between the two tumor assessments showing a response

The Table 8 below shows the summary of duration of response for responder participants as per RECIST 1.1—Responders in activity population

TABLE 8

| | All | |
|---|---|---|
| | (N = 10) | |
| Number of responders^{a} | | |
| Participants with an event [n(%)] | 3 | (30.0) |
| Participants censored [n(%)] | 7 | (70.0) |
| Kaplan-Meier estimates of DOR (months) | NC | (4.337; NC) |

| | |
|---|---|
| Median (95% CI)^{b} | |
| Ranges of DOR (months) | 1.48⁺; 14.52⁺ |
| Min; Max | |

CI: Confidence interval,
CR: Complete response,
PR: Partial response,
SD: Stable disease,
NE: Not evaluable,
NC: Not Calculated
^{a}Including participants with unconfirmed CR or PR.
^{b}Including participants with no post-baseline evaluable tumor assessment but with an early clinical progression.
^{c}Estimated by Clopper Pearson interval.

Confirmation of response (CR/PR) is required: the subsequent tumor assessment used for the confirmation must be assessed at least 4 weeks (>=28 days) after the initial CR/PR assessment. A response can be confirmed if there is a single non-evaluable tumor assessment performed in-between the two tumor assessments showing a response

The Table 9 below shows the summary of progression-free survival as per RECIST 1.1—All-treated population.

TABLE 9

| | All | |
|---|---|---|
| Time to Event or Censoring | (N = 23) | |
| Participants with an event[n(%)] | 9 | (39.1) |
| Participants censored[n(%)] | 14 | (60.9) |
| Kaplan-Meier estimates of PFS(months)^{a} | | |
| 25% quantile (95% CI) | 4.24 | (0.361; 9.002) |
| Median survival (95% CI) | 9.66 | (4.238; NC) |
| 75% quantile (95% CI) | NC | (9.002; NC) |
| PFS Probability (95% CI) ^{b} | | |
| 3 months | 0.752 | (0.503; 0.889) |
| 6 months | 0.608 | (0.337; 0.796) |
| 9 months | 0.608 | (0.337; 0.796) |
| 12 months | 0.405 | (0.145; 0.655) |

| | |
|---|---|
| Number of participants at risk ^{b} | |
| 3 months | 15 |
| 6 months | 7 |
| 9 months | 6 |
| 12 months | 4 |

PFS: Progression-free survival,
CI: Confidence interval,
NC: Not calculated
^{a}Estimated using a log-log transformation of the survival function and the method of Brookmeyer and Crowley.
^{b} Kaplan-Meier estimates. CIs are computed using the log-log transformation of the survival function based and the normal approximation following the Greenwood's formula.

If progression or death is not observed before the analysis cut-off date and prior to the initiation of a further anticancer therapy, then PFS is censored at the date of the last evaluable tumor assessment performed before the analysis cut-off date or date of initiation of a further anticancer therapy, whichever is earlier

If a progression or death is observed after 1 or more non-evaluable tumor assessments, then PFS is not censored and the event is the date of progression, or date of death, whichever is earlier.

All regimens T2, T3 and T4 with tusamitamab ravtansine 150 mg/m² are well tolerated with manageable toxicity and promising response data.

No increase in the safety profile from the standard of care pembrolizumab+platinum-based chemotherapy+pemetrexed is observed.

Regimens T2 and T3 with tusamitamab ravtansine 170 mg/m² are well tolerated with manageable toxicity.

### Example 2. Activity of the Immunoconjugate huMAb2-3-SPDB-DM4 and the Anti-muPD-1 Antibody, as Single Agents or in Combination Against Subcutaneous Colon MC38 Syngeneic Tumor Model in C57BI/6 Mice

Preclinical studies of PD-1 and PD-L1 blockade have relied heavily on mouse syngeneic tumor models with intact immune systems, which facilitate dissection of immunosuppressive mechanisms in the tumor microenvironment. Commercially developed monoclonal antibodies (mAbs) targeting human PD-L1 and PD-1 may not demonstrate cross-reactive binding to their mouse orthologs, and surrogate anti-mouse antibodies are often used in their place to inhibit these immune checkpoints (Schofield et al, Activity of murine surrogate antibodies for durvalumab and tremelimumab lacking effector function and the ability to deplete regulatory T cells in mouse models of cancer, mAbs, 2021; 13:1). They are functionally equivalent to the therapeutic human antibodies, but they are not strictly equivalent (IgG isotype, level of affinity, biological activity, effector function, . . . ). For preclinical investigations on PD-1 blockade, an anti muPD-1 surrogate mAb, the clone RMP1-14 (rat IgG1 anti-muPD-1) was used.

In addition, CEACAM5 protein is not expressed in rodents and human CEACAM5 engineered murine tumors do not grow in immunocompetent mice, reason why these experiments are conducted with the huMAb2-3-SPDB-DM4 ADC, which, at dose high enough, to deliver the payload in a non-specific manner. It will be administered at high dose to exploit the enhanced permeability and retention effect observed for solid tumors subcutaneously implanted in mice that leads to selective delivery of macromolecular drugs to the tumor site. Dose have been modulated to obtain different levels of antitumoral activity (inactivity, activity and/or high activity).

### Experimental Procedure

The activity of huMAb2-3-SPDB-DM4 and the anti-muPD-1 antibody, was evaluated as single agent or in combination in a subcutaneous colon MC38 syngeneic tumor, implanted s.c. in female C57BI/6 mice. Control groups were left untreated. The doses of the compounds used are given in mg/kg.

Mice were randomized in 6 groups (n=6) on day 10 post tumour implantation when median tumour burden reached about 135 mm³. huMAb2-3-SPDB-DM4 was administered at 15 and 25 mg/kg following a single IV administration on day 10 and the anti-muPD-1 antibody (clone RMP1-14) was administered at 10 mg/kg following IV administrations on days 10, 14, 17 and 21.

For the evaluation of anti-tumor activity, animals were weighed, and tumors were measured by caliper 2 times weekly. A dosage producing a 20% weight loss at nadir (mean of group) or 10% or more drug deaths, was considered an excessively toxic dosage. Animal body weights included the tumor weights. Tumor volumes were calculated using the formula mass (mm³)=[length (mm)×width (mm)×width (mm)]/2. The primary efficacy end points are ΔT/ΔC, percent median regression, partial and complete regressions (PR and CR).

Changes in tumor volume for each treated (T) and control (C) are calculated for each tumor by subtracting the tumor volume on the day of first treatment (staging day) from the tumor volume on the specified observation day. The median ΔT is calculated for the treated group and the median ΔC is calculated for the control group. Then the ratio ΔT/ΔC is calculated and expressed as a percentage: ΔT/ΔC=(delta T/delta C)×100.

The dose is considered as therapeutically active when ΔT/ΔC is lower than 40% and very active when ΔT/ΔC is lower than 10%. If ΔT/ΔC is lower than 0, the dose is considered as highly active, and the percentage of regression is dated (Plowman J, Dykes D J, Hollingshead M, Simpson-Herren L and Alley M C. Human tumor xenograft models in NCI drug development. In: Feibig H H B A, editor. Basel: Karger.; 1999 p 101-125):

% tumor regression is defined as the % of tumor volume decrease in the treated group at a specified observation day compared to its volume on the first day of first treatment.

At a specific time point and for each animal, % regression is calculated. The median % regression is then calculated for the group:

$$\% regression ( at t ) = \frac{{volume}_{t 0} - {volume}_{t}}{{volume}_{t 0}} \times 100$$

Partial regression (PR): Regressions are defined as partial if the tumor volume decreases to 50% of the tumor volume at the start of treatment.

Complete regression (CR): Complete regression is achieved when tumor volume=0 mm³ (CR is considered when tumor volume cannot be recorded).

### Results

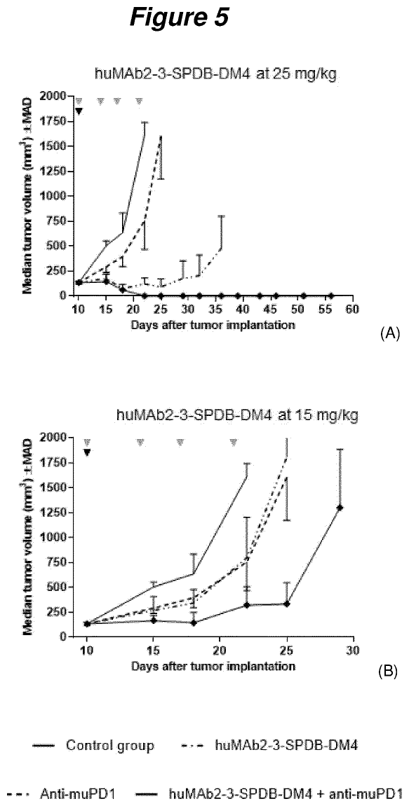

The results for efficacy evaluation of the immunoconjugate huMAb2-3-SPDB-DM4 and the anti-muPD-1 antibody, as single agents or in combination against subcutaneous colon MC38 syngeneic tumor model in C57BI/6 mice are presented on FIG. 5 and Table 10.

HuMAb2-3-SPDB-DM4 and anti-muPD-1 mAb were administered at doses lower than maximal tolerated dose (MTD) and treatments were well tolerated and did not induce toxicity.

The huMAb2-3-SPDB-DM4 as a single agent was highly active at 25 mg/kg with a ΔT/ΔC inferior to 0% (p<0.0001 vs control), a tumor regression of 43%, 4/6 PR and 1/6 CR and inactive at 15 mg/kg with a ΔT/ΔC equal to 46% (no significant vs control).

The anti-muPD-1 mAb as single agent was inactive with a ΔT/ΔC equal to 42% (no significant vs control).

The combination between huMAb2-3-SPDB-DM4 at 25 mg/kg and anti-muPD-1 mAb was highly active with a ΔT/ΔC inferior to 0% (p<0.0001 vs control), a tumor regression of 100%, 5/6 PR and 5/6 CR.

The combination between huMAb2-3-SPDB-DM4 at 15 mg/kg and anti-muPD-1 mAb was active with a ΔT/ΔC equal to 12% (p=0.0030 vs control), 2/6 PR and 1/6 CR

In conclusion to the experiment in the colon MC38 syngeneic tumor, huMAb2-3-SPDB-DM4, when it was active, synergized with anti-muPD-1 mAb in despite of the lack of activity of anti-muPD-1 mAb as single agent leading to complete tumor regression; when both huMAb2-3-SPDB-DM4 and anti-muPD-1 mAb were inactive as single agent, the combination led to a robust activity.

TABLE 10
Activity of huMAb2-3-SPDB-DM4 and anti-muPD-1 (clone RPM1-14),
as single agent or in combination in a subcutaneous colon MC38
syngeneic tumor, implanted s.c. in female C57BI/6 mice

| | | Dosage | Schedule | Drug |
|---|---|---|---|---|
| | | in mg/kg | in | death |
| Agent | Route | (total) | day | (day) |
| huMAb2-3- | IV | 25 (25) | 10 | 0/6 |
| SPDB-DM4 | | 15 (15) | 10 | 0/6 |
| anti-muPD- | IV | 10 (40) | 10, 14, | 0/6 |
| 1 (RPM1- | | | 17, 18 | |
| 14 cl) | | | | |
| huMAb2-3- | IV | 25 (25) | 10 | 0/6 |
| SPDB-DM4 + | | 10 (40) | 10, 14, | |
| anti- | | | 17, 18 | |
| muPD-1 | IV | 15 (15) | 10 | 0/6 |
| | | 10 (40) | 10, 14, | |
| | | | 17, 18 | |
| Control | — | — | — | — |

| | Mean body | | | | | | |
|---|---|---|---|---|---|---|---|
| | weight change | | Median % of | | | Biosatitic | |
| | in % at | Median ΔT/ΔC | regression | Regression | | p value^{a} | Biological |
| Agent | nadir (day) | in % (D 22) | (day) | PR | CR | (D 22) | comments |
| huMAb2-3- | +1.30 | <0 | 43% (D 25) | 4/6 | 1/6 | <0.0001 | Highly |
| SPDB-DM4 | (D 14) | | | | | | active |
| | +3.95 | 46 | — | 0/6 | 0/6 | ns | Inactive |
| | (D 14) | | | | | | |
| anti-muPD- | +0.07 | 42 | — | 0/6 | 0/6 | ns | Inactive |
| 1 (RPM1- | (D 14) | | | | | | |
| 14 cl) | | | | | | | |
| huMAb2-3- | +1.38 | <0 | 100 (D 22) | 5/6 | 5/6 | <0.0001 | Highly |
| SPDB-DM4 + | (D 17) | | | | | | active |
| anti- | +2.13 | 12 | — | 2/6 | 1/6 | =0.0030 | Active |
| muPD-1 | (D 17) | | | | | | |
| Control | −0.75 | — | — | — | — | — | — |
| | (D 14) | | | | | | |

^{a}Statistical analysis. The p-values were obtained using a contrast analysis to compare each treated group versus control using Bonferroni-Holm adjustment for multiplicity after a two-way Anova-Type with repeated measures on tumor volume changes from baseline. A probability less than 5% (p < 0.05) was considered as significant.
ΔT/ΔC = ratio of medians of tumor volume changes from baseline between treated and control groups;
PR = Partial regression;
CR = Complete regression

### Example 3: Activity of the Immunoconjugate huMAb2-3-SPDB-DM4 and the Anti-Mu/huPD-L1 Antibody, as Single Agents or in Combination Against Subcutaneous Colon MC38 Syngeneic Tumor Model in C57BI/6 Mice

For these preclinical studies on combination with PD-L1 blockade, an anti-huPD-L1 mAb, Atezolizumab, that is able to bind and blocks mouse PD-L1 (Magiera-Mularz et al, Human and mouse PD-L1: similar molecular structure, but different druggability profiles. iScience 2021; 24), was used.

### Experimental Procedure

The activity of huMAb2-3-SPDB-DM4 and the anti-mu/huPD-L1 antibody, was evaluated as single agent or in combination in a subcutaneous colon MC38 syngeneic tumor, implanted s.c. in female C57BI/6 mice. Control groups were left untreated. The doses of the compounds used are given in mg/kg.

Mice were randomized in 6 groups (n=6) on day 10 post tumour implantation when median tumour burden reached about 135 mm3. huMAb2-3-SPDB-DM4 was administered at 15 and 25 mg/kg following a single IV administration on day 10 and the anti-mu/huPD-L1 antibody was administered at 10 mg/kg following IV administrations on days 10, 14, 17 and 21.

See above (Ex 2) for the conditions of anti-tumor activity and toxicity evaluation.

### Results

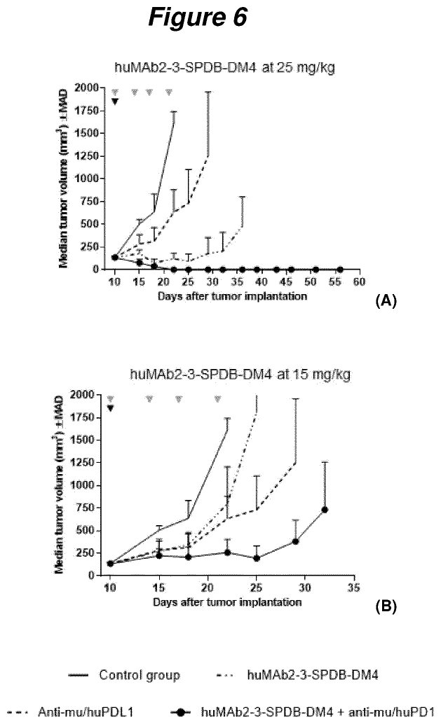

The results for efficacy evaluation of the immunoconjugate huMAb2-3-SPDB-DM4 and the anti-mu/huPD-L1 antibody, as single agents or in combination against subcutaneous colon MC38 syngeneic tumor model in C57BI/6 mice are presented on FIG. 6 and Table 11.

HuMAb2-3-SPDB-DM4 and anti-mu/huPD-L1 mAb were administered at doses lower than maximal tolerated dose (MTD) and treatments were well tolerated and did not induce toxicity.

The huMAb2-3-SPDB-DM4 as a single agent was highly active at 25 mg/kg with a ΔT/ΔC inferior to 0% (p<0.0001 vs control), a tumor regression of 43%, 4/6 PR and 1/6 CR and inactive at 15 mg/kg with a ΔT/ΔC equal to 46% (no significant vs control).

The anti-mu/PD-L1 mAb as single agent was marginally active with a ΔT/ΔC equal to 35% (no significant vs control), 1/6 PR and 1/6 CR.

The combination between huMAb2-3-SPDB-DM4 at 25 mg/kg and anti-mu/huPD-L1 mAb was highly active with a ΔT/ΔC inferior to 0% (p<0.0001 vs control), a tumor regression of 100%, 5/6 PR and 5/6 CR.

The combination between huMAb2-3-SPDB-DM4 at 15 mg/kg and anti-mu/huPD-L1 mAb was very active with a ΔT/ΔC equal to 8% (p=0.0004 vs control) and 1/6 PR

In conclusion to the experiment in the colon MC38 syngeneic tumor, huMAb2-3-SPDB-DM4, when it was active, synergized with anti-mu/huPD-L1 mAb in despite of the marginal activity of anti-mu/huPD-L11 mAb as single agent leading to complete tumor regression; when both huMAb2-3-SPDB-DM4 and anti-mu/huPD-L1 mAb were inactive or marginally active as single agent, the combination led to a robust activity.

TABLE 11
Activity of huMAb2-3-SPDB-DM4 and anti-mu/huPD-L1 (Atezolizumab),
as single agent or in combination in a subcutaneous colon MC38
syngeneic tumor, implanted s.c. in female C57BI/6 mice

| | | Dosage | | Drug |
|---|---|---|---|---|
| | | in mg/kg | Schedule | death |
| Agent | Route | (total) | in day | (day) |
| huMAb2-3- | IV | 25 (25) | 10 | 0/6 |
| SPDB-DM4 | | 15 (15) | 10 | 0/6 |
| anti- | IV | 10 (40) | 10, 14, | 0/6 |
| mu/huPD-L1 | | | 17, 18 | |
| (Atezolizumab) | | | | |
| huMAb2-3- | IV | 25 (25) | 10 | 0/6 |
| SPDB-DM4 + | | 10 (40) | 10, 14, | |
| anti- | | | 17, 18 | |
| mu/huPD-L1 | IV | 15 (15) | 10 | 0/6 |
| | | 10 (40) | 10, 14, | |
| | | | 17, 18 | |
| Control | — | — | — | — |

| | Mean body | | | | | | |
|---|---|---|---|---|---|---|---|
| | weight change | | Median % of | | | Biosatitic | |
| | in % at | Median ΔT/ΔC | regression | Regression | | p value^{a} | Biological |
| Agent | nadir (day) | in % (D 22) | (day) | PR | CR | (D 22) | comments |
| huMAb2-3- | +1.30 | <0 | 43% (D 25) | 4/6 | 1/6 | <0.0001 | Highly |
| SPDB-DM4 | (D 14) | | | | | | active |
| | +3.95 | 46 | — | 0/6 | 0/6 | ns | Inactive |
| | (D 14) | | | | | | |
| anti- | +2.08 | 35 | — | 1/6 | 1/6 | ns | Marginally |
| mu/huPD-L1 | (D 14) | | | | | | active |
| (Atezolizumab) | | | | | | | |
| huMAb2-3- | +1.93 | <0 | 100 (D 22) | 5/6 | 5/6 | <0.0001 | Highly |
| SPDB-DM4 + | (D 14) | | | | | | active |
| anti- | +1.92 | 8 | — | 1/6 | 0/6 | =0.0004 | Very |
| mu/huPD-L1 | (D 15) | | | | | | active |
| Control | −0.75 | — | — | — | — | — | — |
| | (D 14) | | | | | | |

^{a}Statistical analysis. The p-values were obtained using a contrast analysis to compare each treated group versus control using Bonferroni-Holm adjustment for multiplicity after a two-way Anova-Type with repeated measures on tumor volume changes from baseline. A probability less than 5% (p < 0.05) was considered as significant.
ΔT/ΔC = ratio of medians of tumor volume changes from baseline between treated and control groups;
PR = Partial regression;
CR = Complete regression

1. A combination of (i) an antibody-drug conjugate (ADC) comprising an anti-CEACAM5 antibody and (ii) an anti-PD-1 antibody or anti-PD-L1 antibody, in an effective amount, for use in the treatment of a cancer in a subject in need thereof, wherein the cancer expresses CEACAM5.

2. The combination for use according to claim 1, wherein the anti-CEACAM5 antibody comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

3. The combination for use according to claim 1 or 2, wherein the anti-CEACAM5 antibody comprises a variable domain of a heavy chain (VH) consisting of SEQ ID NO: 6 and a variable domain of a light chain (VL) consisting of SEQ ID NO: 7.

4. The combination for use according to any one of claims 1 to 3, wherein the anti-CEACAM5 antibody is tusamitamab.

5. The combination for use according to any one of claims 1 to 4, wherein the ADC comprises at least one cytotoxic agent.

6. The combination for use according to claim 5, wherein the cytotoxic agent is selected from the group consisting of radioisotopes, protein toxins, small molecule toxins, and any combination thereof.

7. The combination for use according to claim 6, wherein the small molecule toxin is selected from the group consisting of antimetabolites, DNA-alkylating agents, DNA-cross-linking agents, DNA-intercalating agents, anti-microtubule agents, topoisomerase inhibitors, and any combination thereof.

8. The combination for use according to claim 7, wherein the anti-microtubule agent is selected from the group consisting of taxanes, vinca alkaloids, maytansinoids, colchicine, podophyllotoxin, griseofulvin, and any combination thereof.

9. The combination for use according to any one of claims 5 to 8, wherein the cytotoxic agent is a maytansinoid selected from the group consisting of N2′-deacetyl-N2′-(3-mercapto-1-oxopropyl)-maytansine (DM1), N2′-deacetyl-N2′(4-methyl-4-mercapto-1-oxopentyl)-maytansine (DM4), and any combination thereof.

10. The combination for use according to any one of claims 5 to 9, wherein the anti-CEACAM5 antibody is covalently attached via a cleavable or non-cleavable linker to the at least one cytotoxic agent.

11. The combination for use according to claim 10, wherein said linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

12. The combination for use according to any one of claims 1 to 11, wherein the ADC is characterized by a drug-to-antibody ratio (DAR) ranging from 1 to 10.

13. The combination for use according to any one of claims 1 to 12, wherein the ADC is tusamitamab ravtansine.

14. The combination for use according to any one of claims 1 to 13, wherein the cancer expresses CEACAM5 with moderate or high intensity defined by immunohistochemistry.

15. The combination for use according to any one of claims 1 to 14, wherein the cancer expresses CEACAM5 with moderate intensity (immunohistochemistry intensity ≥2+ in ≥1% to <50% of tumor cells).

16. The combination for use according to any one of claims 1 to 14, wherein the cancer expresses CEACAM5 with high intensity (immunohistochemistry intensity ≥2+ in ≥50% of tumor cells).

17. The combination for use according to any one of claims 1 to 16, wherein the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g cholangiacarcinoma), prostate cancer, and skin cancer.

18. The combination for use according to claim 17, wherein the cancer is gastric cancer, gastroesophageal junction cancer, or esophageal cancer.

19. The combination for use according to claim 17, wherein the cancer is lung cancer.

20. The combination for use according to claim 19, wherein the lung cancer is nonsquamous non-small cell lung cancer (NSQ NSCLC).

21. The combination for use according to claim 20, wherein the subject has advanced or metastatic NSQ NSCLC.

22. The combination for use according to claim 20, wherein the subject has NSQ NSCLC with no epidermal growth factor receptor (EGFR) sensitizing mutation or v-raf murine sarcoma viral oncogene homolog B1 (BRAF) mutation or anaplastic lymphoma kinase/c-ros oncogene 1 (ALK/ROS) alterations.

23. The combination for use according to any one of claims 1 to 22, wherein the subject has received no prior systemic chemotherapy for treatment of the cancer.

24. The combination for use according to any one of claims 1 to 23, wherein the anti-PD-1 antibody is selected from the group consisting of pembrolizumab, nivolumab, cemiplimab, sintilimab, dostarlimab, and tislelizumab.

25. The combination for use according to any one of claims 1 to 24, wherein the anti-PD-1 antibody is pembrolizumab.

26. The combination for use according to any one of claims 1 to 23, wherein the anti-PD-L1 antibody is selected from the group consisting of atezolizumab, avelumab, and durvalumab.

27. The combination for use according to any one of claims 1 to 26, wherein the anti-PD-1 antibody or the anti-PD-L1 antibody and the ADC are administered sequentially.

28. The combination for use according to any one of claims 1 to 27, wherein the anti-PD-1 antibody or the anti-PD-L1 antibody is administered before the ADC.

29. The combination for use according to any one of claims 1 to 26, wherein the anti-PD-1 antibody or the anti-PD-L1 antibody and the ADC are administered simultaneously.

30. The combination for use according to any one of claims 1 to 29, wherein anti-PD-1 antibody or the anti-PD-L1 antibody is administered to the subject intravenously in a dose of about 200 mg to about 400 mg.

31. The combination for use according to claim 30, wherein the anti-PD-1 antibody or the anti-PD-L1 antibody is administered to the subject intravenously in a dose selected from 200 mg, 350 mg, 360 mg, and 400 mg.

32. The combination for use according to any one of claims 13 to 31, wherein the tusamitamab ravtansine is administered to the subject intravenously in a dose of about 60 mg/m² to about 190 mg/m².

33. The combination for use according to claim 32, wherein the tusamitamab ravtansine is administered to the subject intravenously in a dose of about 120 mg/m² to about 170 mg/m².

34. The combination for use according to any one of claims 30 to 33, wherein the anti-PD-1 antibody or the anti-PD-L1 antibody is administered to the subject intravenously in a dose of about 200 mg and the tusamitamab ravtansine is administered to the subject intravenously in a dose of about 120 mg/m² to about 170 mg/m².

35. The combination for use according to claim 34, wherein the anti-PD-1 antibody or the anti-PD-L1 antibody and the tusamitamab ravtansine are administered once every three weeks.

36. The combination for use according to any one of claims 30-35, wherein the anti-PD-1 antibody or the anti-PD-L1 antibody is administered to the subject intravenously in a dose of about 400 mg and the tusamitamab ravtansine is administered to the subject intravenously in a dose of about 120 mg/m² to about 170 mg/m².

37. The combination for use according to claim 36, wherein the anti-PD-1 antibody or the anti-PD-L1 antibody is administered once every six weeks and the tusamitamab ravtansine is administered once every three weeks.

38. The combination for use according to any one of claims 1 to 37, wherein the ADC is tusamitamab ravtansine and the anti-PD-1 antibody is pembrolizumab.

39. The combination for use according to any one of claims 1 to 36, the anti-PD-1 antibody or the anti-PD-L1 antibody and the ADC are administered about once every three weeks, the ADC being tusamitamab ravtansine and being administered to the subject intravenously in a dose of about 120 mg/m².

40. The combination for use according to any one of claims 1 to 36, the anti-PD-1 antibody or the anti-PD-L1 antibody and the ADC are administered about once every three weeks, the ADC being tusamitamab ravtansine and being administered to the subject intravenously in a dose of about 150 mg/m².

41. The combination for use according to claim 39 or 40, wherein the anti-PD-1 antibody is pembrolizumab.

42. The combination for use according to any one of claims 39 to 41, wherein the anti-PD-1 antibody is administered to the subject intravenously in a dose of about 200 mg.

43. The combination for use according to any one of claims 1 to 38, further comprising administering (iii) a platinum-based chemotherapy to the subject.

44. The combination for use according to any one of claims 1 to 38 and 41, wherein the platinum-based chemotherapy is selected from the group consisting of cisplatin and carboplatin.

45. The combination for use according to any one of claims 1 to 38, 41 and 42, wherein the anti-PD-1 antibody or the anti-PD-L1 antibody is administered before the ADC and the platinum-based chemotherapy.

46. The combination for use according to any one of claims 1 to 38 and 41 to 43, wherein the anti-PD-1 antibody or the anti-PD-L1 antibody, the ADC, and the platinum-based chemotherapy are administered to the subject for at least four cycles.

47. The combination for use according to any one of claims 1 to 38 and 41 to 44, wherein the use comprises administering cisplatin to the subject.

48. The combination for use according to claim 45, wherein the cisplatin is administered intravenously to the subject in a dose from 38 mg/m² to 75 mg/m².

49. The combination for use according to claim 45, wherein the cisplatin is administered intravenously to the subject in a dose of about 75 mg/m².

50. The combination for use according to any one of claims 1 to 38 and 41 to 44, wherein the use comprises administering carboplatin to the subject.

51. The combination for use according to claim 48, wherein the carboplatin is administered intravenously to the subject in a dose of about (target AUC)×[(140−age)×(weight in kg)/serum creatinine (mg/dL)×72(×0.85 if female)+25], wherein the target AUC is from AUC 2.5 to AUC 5.

52. The combination for use according to claim 49, wherein the target AUC is AUC 5.

53. The combination of any one of the preceding claims, further comprising administering pemetrexed to the subject.

54. The combination for use according to claim 51, wherein the pemetrexed is administered intravenously at a dose from 250 mg/m² to 500 mg/m².

55. The combination for use according to claim 52, wherein the pemetrexed is administered intravenously at a dose about 500 mg/m².

56. The combination for use according to any of claims 51 to 53, wherein the pemetrexed is administered intravenously after a vitamin supplementation.

57. The combination for use according to any one of claims 51 to 54, the anti-PD-1 antibody or the anti-PD-L1 antibody and the ADC are administered about once every three weeks, the ADC being tusamitamab ravtansine and being administered to the subject intravenously in a dose of about 120 mg/m².

58. The combination for use according to any one of claims 51 to 54, the anti-PD-1 antibody or the anti-PD-L1 antibody and the ADC are administered about once every three weeks, the ADC being tusamitamab ravtansine and being administered to the subject intravenously in a dose of about 150 mg/m².

59. The combination for use according to claim 57 or 58, wherein the anti-PD-1 antibody is pembrolizumab.

60. The combination for use according to any one of claims 57 to 59, wherein the anti-PD-1 antibody is administered to the subject intravenously in a dose of about 200 mg.
